(19) United States
(12) Patent Application Publication
(10) Pub. No.: US 2009/0263322 A1
(43) Pub. Date: Oct. 22, 2009

(54) OCTAHYDRO-INDOLIZINE AND QUINOLIZINE AND HEXAHYDRO-PYRROLIZINES

(76) Inventors: Richard Apodaca, San Diego, CA (US); Nicholas I. Carruthers, Poway, CA (US); John R. Carson, Norristown, PA (US); Wenying Chai, San Diego, CA (US); Annette K. Kwok, San Diego, CA (US); Xiaobing Li, Belle mead, NJ (US); Timothy W. Lovenberg, San Diego, CA (US); Dale A. Rudolph, San Diego, CA (US); Chandravadan R. Shah, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/494,754

(22) Filed: Jun. 30, 2009

Related U.S. Application Data
(63) Continuation of application No. 11/205,958, filed on Aug. 17, 2005, now abandoned.
Application No. 11/205,958 is a continuation of application No. 10/773,808, filed on Feb. 6, 2004, now abandoned.
Application No. 10/773,808 is a continuation of application No. 09/960,031, filed on Sep. 21, 2001, now abandoned.
(60) Provisional application No. 60/234,504, filed on Sep. 22, 2000.
(60) Provisional application No. 60/234,505, filed on Sep. 22, 2000.

(51) Int. Cl. A61K 31/437; C07D 221/04; C07D 401/14; A61K 31/496; A61K 51/00; A61P 11/00; A61P 25/00

(52) U.S. Cl. 424/1.89; 546/112; 544/362; 514/299; 514/253.04; 424/1.81

(57) ABSTRACT

The invention features substituted fused bicyclic compounds, pharmaceutical compositions containing them, and methods of using them to treat or prevent histamine-mediated diseases and conditions.

### FIELD OF THE INVENTION

The present invention relates to octahydro-indolizine and quinolizine and hexahydro-pyrrolizine derivatives, their synthesis and their use, for example, for the treatment of disorders and conditions mediated by the histamine receptor.

### BACKGROUND OF THE INVENTION

Histamine [2-(imidazol-4-yl)ethylamine] is a transmitter substance. Histamine exerts a physiological effect via multiple distinct G-protein coupled receptors. It plays a role in immediate hypersensitivity reactions and is released from mast cells following antigen IgE antibody interaction. The actions of released histamine on the vasculature and smooth muscle system account for the symptoms of the allergic response. These actions occur at the H₁ receptor (Ash, A. S. F. and Schild, H. O., Br. J. Pharmacol., 1966, 27, 427) and are blocked by the classical antihistamines (e.g. diphenhydramine). Histamine is also an important regulator of gastric acid secretion through its action on parietal cells. These effects of histamine are mediated via the H₂ receptor (Black, J. W., Duncan, W. A. M., Durant, C. J., Ganellin, C. R. and Parsons, E. M., Nature, 1972, 236, 385) and are blocked by H₂ receptor antagonists (e.g. cimetidine). The third histamine receptor —H₃— was first described as a presynaptic autoreceptor in the central nervous system (CNS) (Arrang, J.-M., Garbarg, M., and Schwartz, J.-C., Nature 1983, 302, 832) controlling the synthesis and release of histamine. Recent evidence has emerged showing that the H₃ receptors are also located presynaptically as heteroreceptors on serotonergic, noradrenergic, dopaminergic, cholinergic, and GABAergic (gamma-aminobutyric acid containing) neurons. These H₃ receptors have also recently been identified in peripheral tissues such as vascular smooth muscle. Consequently there are many potential therapeutic applications for histamine H₃ agonists, antagonists, and inverse agonists. (See: “The Histamine H₃ Receptor-A Target for New Drugs”, Leurs, R., and Timmerman, H., (Editors), Elsevier, 1998; Morisset et al., Nature, 2000, 408, 860-864.) A fourth histamine receptor —H₄— was recently described by Oda et al., (J. Biol. Chem., 2000, 275, 36781-36786).

The potential use of histamine H₃ agonists in sleep/wake and arousal/vigilance disorders is suggested based on animal studies (Lin et al, Br. Res., 1990, 523, 325; Monti et al Eur. J. Pharmacol., 1991, 205, 283). Their use in the treatment of migraine has also been suggested (McLeod et al Abstr. Society Neuroscience, 1996, 22, 2010) based on their ability to inhibit neurogenic inflammation. Other applications could be a protective role in myocardial ischemia and hypertension where blockade of norepinephrine release is beneficial (Imamura et al J. Pharmacol. Expt. Ther., 1994, 271, 1259). It has been suggested that histamine H₃ agonists may be beneficial in asthma due to their ability to reduce non-adrenergic non-cholinergic (NANC) neurotransmission in airways and to reduce microvascular leakage (Ichinose et al Eur. J. Pharmacol., 1989, 174, 49).

Several indications for histamine H₃ antagonists and inverse agonists have similarly been proposed based on animal pharmacology experiments with known histamine H₃ antagonists (e.g. thioperamide). These include dementia, Alzheimer's disease (Panula et al Abstr. Society Neuroscience, 1995, 21, 1977), epilepsy (Yokoyama et al Eur. J. Pharmacol., 1993, 234, 129) narcolepsy, eating disorders (Machidori et al Brain Research 1992, 590, 180), motion sickness, vertigo, attention deficit hyperactivity disorders (ADHD), learning and memory (Barnes et al Abstr. Society Neuroscience, 1993, 19, 1813), schizophrenia (Schlicker et al Naunyn-Schmiedeberg's Arch. Pharmacol., 1996, 353, 290-294); (also see; Stark et al Drugs Future, 1996, 21, 507 and Leurs et al Progress in Drug Research, 1995, 45, 107 and references cited therein). Histamine H₃ antagonists, alone or in combination with a histamine H₁ antagonist, are reported to be useful for the treatment of upper airway allergic response (U.S. Pat. Nos. 5,217,986; 5,352,707 and 5,869,479). Recently, a histamine H₃ antagonist (GT-2331) was identified and is being developed by Gliatech Inc. (Gliatech Inc. Press Release Nov. 5, 1998; Bioworld Today, Mar. 2, 1999) for the treatment of CNS disorders.

As noted, the prior art related to histamine H₃ ligands has been comprehensively reviewed (“The Histamine H₃ Receptor-A Target for New Drugs”, Leurs, R., and Timmerman, H., (Editors), Elsevier, 1998). Within this reference the medicinal chemistry of histamine H₃ agonists and antagonists was reviewed (see Krause et al and Phillips et al respectively). The importance of an imidazole moiety containing only a single substitution in the 4 position was noted together with the deleterious effects of additional substitution on activity. Particularly methylation of the imidazole ring at any of the remaining unsubstituted positions was reported to strongly decrease activity. Additional publications support the hypothesis that an imidazole function is essential for high affinity histamine H₃ receptor ligands (See, Ali et al J. Med. Chem., 1999, 42, 903 and Stark et al, Drugs Future, 1996, 21, 507 and references cited therein). However many imidazole containing compounds are substrates for histamine methyl transferase, the major histamine metabolizing enzyme in humans, which leads to shortened half lives and lower bioavailability (See, Rouleau et al J. Pharmacol. Exp. Ther. 1997, 281, 1085). In addition, imidazole containing drugs, via their interaction with the cytochrome P450 monooxygenase system, can result in unfavorable biotransformations due to enzyme induction or enzyme inhibition. (Kapetanovic et al Drug Metab. Dispos. 1984, 12, 560; Sheets et al Drug Metab. Dispos. 1984, 12, 603; Back, et al Br. J. Pharmacol. 1985, 85, 121; Lavrijsen et al Biochem. Pharmacol. 1986, 35, 1867; Drug Saf., 1998, 18, 83). The poor blood brain barrier penetration of earlier histamine H₃ receptor ligands may also be associated with the imidazole fragment (Ganellin et al Arch. Pharm. (Weinheim, Ger.) 1998, 331, 395).

More recently, several publications have described histamine H₃ ligands that do not contain an imidazole moiety. For example; Ganellin et al Arch. Pharm. (Weinheim, Ger.) 1998, 331, 395; Walczynski et al Arch. Pharm. (Weinheim, Ger.) 1999, 332, 389; Walczynski et al Farmaco 1999, 684; Linney et al J. Med. Chem. 2000, 2362; Tozer and Kalindjian Exp. Opin. Ther. Patents 2000, 10, 1045-1055; U.S. Pat. No. 5,352,707; PCT Application WO99/42458, Aug. 26, 1999; and European Patent Application 0978512, Feb. 9, 2000.

The compounds of the present invention do not contain the imidazole moiety, and its inherent liabilities, and maintain potency at the human H₃ receptor. Thus in the present invention receptor binding was determined using the human histamine H₃ receptor (See Lovenberg et al Mol. Pharmacol. 1999, 1107). Screening using the human receptor is particularly important for the identification of new therapies for the treatment of human disease. Conventional binding assays for example are determined using rat synaptosomes (Garbarg et al J. Pharmacol. Exp. Ther. 1992, 263, 304), rat cortical membranes (West et al Mol. Pharmacol. 1990, 610), and guinea pig brain (Korte et al Biochem. Biophys. Res. Commun. 1990, 978). Only limited studies have been performed previously using human tissue but these allude to significant differences in the pharmacology of rodent and primate receptors (West et al Eur. J. Pharmacol. 1999, 233).

We now describe a series of octahydro-indolizine and quinolizine and hexahydro-pyrrolizine derivatives with the ability to modulate the activity of the histamine receptor, specifically the H₃ receptor, without the inherent problems associated with the presence of an imidazolyl moiety.

Substituted octahydroindolizine compounds useful as analgesics are previously described in; Carmosin, R. J.; Carson, J. R. “Octahydroindolizine Compounds Useful as Analgesics”, U.S. Pat. No. 4,582,836, 1986; Carmosin, R. J.; Carson, J. R. “3-Diphenyl Substituted Octahydroindolizine Analgesic Compounds”, U.S. Pat. No. 4,683,239, 1987; Carmosin, R. J.; Carson, J. R. “5-Substituted Octahydroindolizine Analgesics Compounds and 7-Keto Intermediates”, U.S. Pat. No. 4,689,329, 1987, and Carson, J. R.; Carmosin, R. J.; Vaught, J. L.; Gardocki, J. F.; Costanzo, M. J.; Raffa, R. B.; Almond, H. R. J. Med. Chem. 1992, 35, 2855-2863. Octahydroquinolizines as analgesics are previously described in; Carmosin, R. J.; Carson, J. R. “4-Substituted Octahydroquinolizine Analgesic Compounds and Octahydroquinolizinium Intermediates”, U.S. Pat. No. 4,716,172, 1987. Pyrrolizine analgesics are previously described in; Carmosin, R. J.; Carson, J. R. “Hexahydropyrrolizines Compounds Useful as Analgesics”, U.S. Pat. No. 4,800,207,1989.

### SUMMARY OF THE INVENTION

The invention features compounds of the formula (IA):

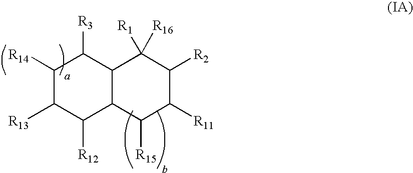

wherein:

a is 0 and b is 0;

or a is 1 and b is 0;

or a is 1 and b is 1;

Y is selected from N and N→O;

one of R₁, R₂ and R₃ is a ring moiety selected from C₄₋₆ cycloalkyl, phenyl, naphthyl, C₁₋₅ heterocyclyl, (C₄₋₆ cycloalkyl)C₁₋₃ alkylene, (phenyl)C₁₋₃ alkylene, (naphthyl) C₁₋₃ alkylene, and (C₁₋₅ heterocyclyl)C₁₋₃ alkylene;
and the remaining two of R₁, R₂ and R₃ are independently selected from hydrogen, halogen, and C₁₋₆ alkyl;

wherein said ring moiety is substituted with a moiety of formula:

—X—W-Z, X-Z, W-Z or Z;
wherein X is selected from the group consisting of O, S, SO₂, SO, NR₄, —CH═CH—, —C≡C—, —OCH₂—C≡C—, —C≡C—CH₂O—, —CH(R₅)—, CO, —O—CO—, —CO—O—, CHOH, —NR₄—CO—, —CO—NR₄—, —SO₂—NH—, —NR₄—SO₂—, and —SO₂—NR₄—; R₄ is H, or C₁₋₆ alkyl; R₅ is H, C₁₋₆ alkyl, or hydroxy;
W is C₁₋₆ alkylene, phenylene, (phenylene)(C₁₋₃ alkylene), or —CH₂—CHCH—CH₂—;
Z is selected from:
(i) NR₂₁R₂₂, NHCOR₂₃, or NHSO₂R₂₃,
(ii) C₃₋₆ heterocyclyl or C₇₋₁₂ fused bicyclyl, and
(iii) phenyl substituted with a C₃₋₆ heterocyclyl group, or with a (C₃₋₆ heterocyclyl)C₁₋₆ alkylene group,
   wherein each phenyl or heterocyclyl group in (ii) or (iii) may be substituted with one to four substituents independently selected from the group consisting of halo, hydroxy, C₁₋₆ alkyl, C₁₋₆ alkoxy, cyclohexyl, cyclohexenyl, phenyl, (phenyl)C₁₋₆ alkylene, trihalo C₁₋₆ alkyl, nitro, SCH₃, NR₂₁R₂₂, amido, amidino, amino C₁₋₆ alkyl, acetylene, CHR₂₃R₂₄, COR₂₃, acetyl, NHCOCH₃, C₃₋₆ heterocyclyl, (C₃₋₆ heterocyclyl) C₁₋₆ alkylene, cyano, NHSO₂CH₃, N(SO₂CH₃)₂, carboxy, C₁₋₆ alkoxycarbonyl, amidoxime, trihalo C₁₋₆ alkoxy, oxo, hydroxyiminomethyl, C₁₋₆ alkylcarboxy, carboxy C₁₋₆ alkyl, trihaloacetyl, and methylsulfonyl;
   wherein each of R₂, and R₂₂ is independently selected from H, C₁₋₆ alkyl, C₄₋₇ cycloalkyl, phenyl, benzyl, C₁₋₆ alkoxy, hydroxy, C₁₋₆ alkylamino, di(C₁₋₆)alkylamino, C₂₋₈ acyl, C₁₋₈ alkylsulfonyl;
   R₂₃ is C₁₋₆ alkyl, C₄₋₇ cycloalkyl, phenyl, benzyl, C₁₋₆ alkoxy, hydroxy, aryl, C₁₋₆ alkylamino, di(C₁₋₆)alkylamino, C₂₋₈ acyl, C₁₋₈ alkylsulfonyl;
   R₂₄ is H, halogen, hydroxy, amino, C₁₋₆ alkyl, C₄₋₇ cycloalkyl, phenyl, or benzyl;

in addition, said R₁, R₂ or R₃ that is a ring moiety is optionally substituted with between 1 and 3 substituents Q₁, Q₂, and Q₃, which, if present, are independently selected from: R₂₅, NR₂₆R₂₇, NHCOR₂₈, NHSOR₂₉, and NHSO₂R₃₀;
wherein R₂₅ is H, C₁₋₆ alkyl, C₄₋₇ cycloalkyl, phenyl, benzyl, C₁₋₆ alkoxy, hydroxy, C₁₋₆ alkylamino, di(C₁₋₆)alkylamino, C₂₋₈ acyl, or C₁₋₈ alkylsulfonyl;
wherein each of R₂₆ and R₂₇ is independently selected from H, C₁₋₆ alkyl, C₄₋₇ cycloalkyl, phenyl, benzyl, C₁₋₆ alkoxy, hydroxy, C₁₋₆ alkylamino, di(C₁₋₆)alkylamino, C₂₋₈ acyl, C₁₋₈ alkylsulfonyl;
each of R₂₈, R₂₉, and R₃₀ is C₁₋₆ alkyl, C₄₋₇ cycloalkyl, phenyl, benzyl, C₁₋₆ alkoxy, hydroxy, C₁₋₆ alkylamino, di(C₁₋₆)alkylamino, C₂₋₈ acyl, C₁₋₈ alkylsulfonyl;
   and
R₁₁, R₁₂, R₁₄ and R₁₅ are each independently selected from hydrogen, halogen, C₁₋₆ alkyl and C₁₋₆ alkoxy;
   R₁₃ is selected from hydrogen, oxo, and phenyl;
   R₁₆ is selected from hydrogen, cyano, C₁₋₆ alkyl, and C₁₋₆ alkylamino;
   wherein each of the above carbocyclyl and heterocarbocyclyls can be optionally substituted with between 1 and 3 substituents selected from C₁₋₄ alkyl, hydroxy, amino, halo, C₁₋₄ alkoxy, CONH₂, phenyl, and C₁₋₄ alkylamino, di(C₁₋₄)alkylamino;

and wherein —X—W-Z is not [4-(imidazol-1-yl)-phenyl]oxy where a is 1 and b is 0;

or a pharmaceutically acceptable salt, ester, or amide thereof.

Multiple stereocenters or chiral centers are possible and both isolated forms and mixtures are encompassed by the invention.

The invention also features a pharmaceutical composition comprising a compound of the invention and a pharmaceutically acceptable carrier; and methods of preparing or formulating such compositions. A composition of the invention may further include more than one compound of the invention, or a combination therapy (combination formulation or administering a combination of differently formulated active agents).

The invention also provides methods of treating certain conditions and diseases, each of which methods includes administering a therapeutically effective (or jointly effective) amount of a compound or composition of the invention to a subject in need of such treatment. The disclosed compounds are useful in methods for treating or preventing neurologic disorders including sleep/wake and arousal/vigilance disorders (e.g. insomnia and jet lag), attention deficit hyperactivity disorders (ADHD), learning and memory disorders, cognitive dysfunction, migraine, neurogenic inflammation, dementia, mild cognitive impairment (pre-dementia), Alzheimer's disease, epilepsy, narcolepsy, eating disorders, obesity, motion sickness, vertigo, schizophrenia, substance abuse, bipolar disorders, manic disorders and depression, as well as other histamine H₃ receptor mediated disorders such as upper airway allergic response, asthma, itch, nasal congestion and allergic rhinitis in a subject in need thereof.

For example, the invention features methods for preventing, inhibiting the progression of, or treating upper airway allergic response, asthma, itch, nasal congestion and allergic rhinitis. In yet another embodiment, the disclosed compounds may be used in a combination therapy method including administering a jointly effective dose of an H₃ antagonist and administering a jointly effective dose of a histamine H₁ antagonist, such as loratidine (CLARITIN™), desloratidine (CLARINEX™), fexofenadine (ALLEGRA™) and cetirizine (ZYRTEC™), for the treatment of allergic rhinitis, nasal congestion and allergic congestion.

In yet another embodiment, the disclosed compounds may be used in a combination therapy method, including administering a jointly effective dose of an H₃ antagonist and administering a jointly effective dose of a neurotransmifter re-uptake blocker, such as a selective serotonin re-uptake inhibitor (SSRI) or a non-selective serotonin, dopamine or norepinephrine re-uptake inhibitor, including fluoxetine (PROZAC™), sertraline (ZOLOFT™), paroxetine (PAXIL™) and amitryptyline, for the treatment of depression, mood disorders or schizophrenia.

Further methods of the invention are: (i) a method for treating one or more disorders or conditions selected from the group consisting of sleep/wake disorders, narcolepsy, and arousal/vigilance disorders, comprising administering to a subject a therapeutically effective amount of a disclosed compound; (ii) a method for treating attention deficit hyperactivity disorders (ADHD), comprising administering to a subject a therapeutically effective amount of a disclosed compound; (iii) a method for treating one or more disorders or conditions selected from the group consisting of dementia, mild cognitive impairment (pre-dementia), cognitive dysfunction, schizophrenia, depression, manic disorders, bipolar disorders, and learning and memory disorders, comprising administering to a subject a therapeutically effective amount of a disclosed compound; (iv) a method for treating or preventing upper airway allergic response, nasal congestion, or allergic rhinitis, comprising administering to a subject a therapeutically effective amount of a disclosed compound; and (v) a method for studying disorders mediated by the histamine H₃ receptor, comprising using a ¹¹C- or 18F-labeled disclosed compound as a positron emission tomography (PET) molecular probe.

Also provided is a method for treating a disorder or condition mediated by the histamine H₃ receptor in a subject, said method comprising administering to a subject a therapeutically effective amount of a disclosed compound. The disorder or condition is selected from the group consisting of sleep/wake disorders, arousal/vigilance disorders, migraine, asthma, dementia, mild cognitive impairment (pre-dementia), Alzheimer's disease, epilepsy, narcolepsy, eating disorders, motion sickness, vertigo, attention deficit hyperactivity disorders, learning disorders, memory retention disorders, schizophrenia, nasal congestion, allergic rhinitis, and upper airway allergic response.

Additional features and advantages of the invention will become apparent from the detailed description and examples below, and the appended claims.

### DETAILED DESCRIPTION OF THE INVENTION

The present invention provides methods for the treatment of disorders and conditions modulated by the histamine receptor, more particularly the H₃ receptor, by administering substituted octahydro-indolizine, quinolizine and pyrrolizine derivatives.

### A. TERMS

Certain terms are defined below and by their usage throughout this disclosure.

As used herein, “halogen” shall mean chlorine, bromine, fluorine and iodine, or monovalent radicals thereof.

As used herein, the term “alkyl”, whether used alone or as part of a substituent group, shall include straight and branched carbon chains. For example, alkyl radicals include methyl, ethyl, propyl, isopropyl, butyl, isobutyl, sec-butyl, t-butyl, pentyl and the like. Unless otherwise noted, “lower” when used with alkyl means a carbon chain composition of 1-4 carbon atoms. “Alkylene” refers to a bivalent hydrocarbyl group, such as methylene (CH₂), ethylene (—CH₂—CH₂—) or propylene (—CH₂CH₂CH₂—).

As used herein, unless otherwise noted, “alkoxy” shall denote an oxygen ether radical of the above described straight or branched chain alkyl groups. For example, methoxy, ethoxy, n-propoxy, sec-butoxy, t-butoxy, n-hexyloxy and the like.

As used herein, unless otherwise noted, “cycloalkyl” shall denote a three- to eight-membered, saturated monocyclic carbocyclic ring structure. Suitable examples include cyclopropyl, cyclobutyl, cyclopentyl, cyclohexyl, cycloheptyl and cyclooctyl.

As used herein, unless otherwise noted, “cycloalkenyl” shall denote a three- to eight-membered, partially unsaturated, monocyclic, carbocyclic ring structure, wherein the ring structure contains at least one double bond. Suitable examples include cyclohexenyl, cyclopentenyl, cycloheptenyl, cyclooctenyl, cyclohex-1,3-dienyl and the like.

As used herein, unless otherwise noted, “aryl” shall refer to carbocyclic aromatic groups such as phenyl, naphthyl, and the like. Divalent radicals include phenylene (—C₆H₄—) which is preferably phen-1,4-diyl, but may also be phen-1,3-diyl.

As used herein, unless otherwise noted, “aralkyl” shall mean any alkyl group substituted with an aryl group such as phenyl, naphthyl and the like. Examples of aralkyls include benzyl, phenethyl, and phenylpropyl.

As used herein, unless otherwise noted, the terms “heterocycle”, “heterocyclyl” and “heterocyclo” shall denote any five-, six-, or seven-membered monocyclic, nine or ten membered bicyclic or thirteen or fourteen membered tricyclic ring structure containing at least one heteroatom moiety selected from the group consisting of N, O, SO, SO₂, (C═O), and S, and preferably N, O, or S, optionally containing one to four additional heteroatoms in each ring. In some embodiments, the heterocyclyl contains between 1 and 3 or between 1 and 2 additional heteroatoms. Unless otherwise specified, a heterocyclyl may be saturated, partially unsaturated, aromatic or partially aromatic. The heterocyclyl group may be attached at any heteroatom or carbon atom which results in the creation of a stable structure.

Exemplary monocyclic heterocyclic groups can include pyrrolidinyl, pyrrolyl, indolyl, pyrazolyl, oxetanyl, pyrazolinyl, imidazolyl, imidazolinyl, imidazolidinyl, oxazolyl, oxazolidinyl, isoxazolinyl, isoxazolyl, thiazaolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolidinyl, 2-oxazepinyl, azepinyl, hexahydroazepinyl, 4-piperidinyl, pyridyl, N-oxo-pyridyl, pyrazinyl, pyrimidinyl, pyridazinyl, tetrahydropyranyl, tetrahydrothiopyranyl, tetrahydrothiopyranyl sulfone, morpholinyl, thiomorpholinyl, thiomorpholinyl sulfoxide, thiomorpholinyl sulfone, 1,3-dixolane and tetrahydro-1,1-dioxothienyl, dioxanyl, isothiazolidinyl, thietanyl, thiiranyl, triazinyl, triazolyl, tetrazolyl, azetidinyl and the like.

For example, where Z is a non-aromatic nitrogen-containing heterocyclyl, preferred values for Z include piperidyl, piperazinyl, pyrrolinyl, pyrrolidinyl, morpholinyl, and N—(C₁₋₆ alkyl)piperazinyl. These may be linked to the rest of the molecule by a nitrogen or a carbon atom; in general, N-linked heterocyclyls are preferred. Z can be substituted with between 1 and 3 substituents selected from pyridyl, pyrimidyl, furyl, thiofuryl, imidazolyl, (imidazolyl)C₁₋₆ alkylene, oxazolyl, thiazolyl, 2,3-dihydro-indolyl, benzimidazolyl, 2-oxobenzimidazolyl, (tetrazolyl)C₁₋₆ alkylene, tetrazolyl, (triazolyl)C₁₋₆ alkylene, triazolyl, (pyrrolyl)C₁₋₆ alkylene, and pyrrolyl. Examples of substituted Z, wherein the substituent comprises a heterocyclyl, include: 4-(4-chloropyridin-2-yl)amino-piperidin-1-yl; 4-(4-chloropyrimidin-2-yl)amino-piperidin-1-yl; 2-([1,2,4]triazol-1-yl)methyl-morpholin-1-yl; 3-(pyrazin-2-yl)piperidin-1-yl; 4-(pyrazol-1-yl)piperidin-1-yl; 4-(pyrimidin-2-yl)piperazin-1-yl; 4-(furan-2-yl)methylpiperazin-1-yl; 4-(thiophen-2-yl)methylpiperazin-1-yl; 4-(4-chloropyridin-2-yl)-[1,4]diazepan-1-yl; and 5-(isoxazol-5-yl)-2,5-diaza-bicyclo[2.2.1]heptan-2-yl.

Exemplary bicyclic heterocyclic groups include benzthiazolyl, benzoxazolyl, benzoxazinyl, benzothienyl, quinuclidinyl, quinolinyl, quinolinyl-N-oxide, tetrahydroisoquinolinyl, isoquinolinyl, benzimidazolyl, benzopyranyl, indolizinyl, benzofuryl, chromonyl, coumarinyl, cinnolinyl, quinoxalinyl, indazolyl, pyrrolopridyl, furopyridinyl (such as furo[2,3-c]pyridinyl, furo[3,1-b]pyridinyl), or furo[2,3-b]pyridinyl), dihydroisoindolyl, dihydroquinazolinyl (such as 3,4-dihydro-4-oxo-quinazolinyl), tetrahydroquinolinyl (such as 1,2,3,4-tetrahydroquinolinyl), tetrahydroisoquinolinyl(such as 1,2,3,4-tetrahydroisoquiunolinyl), benzisothiazolyl, benzisoxazolyl, benzodiazinyl, benzofurazanyl, benzothiopyranyl, benzotriazolyl, benzpyrazolyl, dihydrobenzofuryl, dihydrobenzothienyl, dihydrobenzothiopyranyl, dihydrobenzothiopyranyl sulfone, dihydrobenzopyranyl, indolinyl, isoindolyl, tetrahydroindoazolyl (such as 4,5,6,7-tetrahydroindazolyl), isochromanyl, isoindolinyl, naphthyridinyl, phthalazinyl, piperonyl, purinyl, pyridopyridyl, quinazolinyl, tetrahydroquinolinyl, thienofuryl, thienopyridyl, thienothienyl,

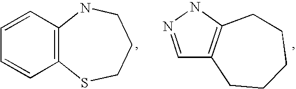

and the like.

Exemplary tricyclic heterocylclic groups include acridinyl, phenoxazinyl, phenazinyl, phenothiazinyl, carbozolyl, perminidinyl, phenanthrolinyl, carbolinyl, naphthothienyl, thianthrenyl, and the like.

Preferred heterocyclyl groups include morpholinyl, piperidinyl, piperazinyl, pyrrolidinyl, pyrimidinyl, pyridyl, pyrrolyl, imidazolyl, oxazolyl, isoxazolyl, acridinyl, azepinyl, hexahydroazepinyl, azetidinyl, indolyl, isoindolyl, thiazolyl, thiadiazolyl, quinolinyl, isoquinolinyl, 1,2,3,4-tetrahydroquinolinyl, 1,3,4-trihydroisoquinolinyl, 4,5,6,7-tetrahydroindadolyl, benzoxazinyl, benzoaxzolyl, benzthiazolyl, benzimidazolyl, tetrazolyl, oxadiazolyl,

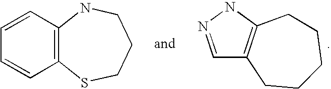

As used herein, unless otherwise noted, the term “heterocyclyl-alkyl” or “heterocyclyl-alkylene” shall denote any alkyl group substituted with a heterocyclyl group, wherein the heterocycly-alkyl group is bound through the alkyl portion to the central part of the molecule. Suitable examples of heterocyclyl-alkyl groups include, but are not limited to piperidinylmethyl, pyrrolidinylmethyl, piperidinylethyl, piperazinylmethyl, pyrrolylbutyl, piperidinylisobutyl, pyridylmethyl, pyrimidylethyl, and the like.

When a particular group is “substituted” (e.g., alkyl, alkylene, cycloalkyl, aryl, heterocyclyl, heteroaryl), that group may have one or more substituents, preferably from one to five substituents, more preferably from one to three substituents, most preferably from one to two substituents, independently selected from the list of substituents. Unless otherwise specified, the substituents are independently selected from hydroxy, halogen, lower alkyl, hydroxyalkyl, alkoxy, trifluoromethyl, amino, dialkylamino, aryl, aralkyl, nitro and the like.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein.

Under standard nomenclature used throughout this disclosure, the terminal portion of the designated side chain is described first, followed by the adjacent functionality toward the point of attachment. Thus, for example, a “phenyl(alkyl)amido(alkyl)” substituent refers to a group of the formula

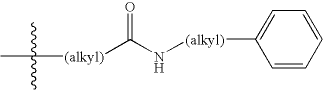

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes prevention, inhibition of onset, or alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

### B. COMPOUNDS

The invention provides the disclosed compounds, such as those of formula (IA) in the Summary section above. Preferred compounds include those wherein:
(a) Y is N;
(b) a is 1 and b is 0;
(c) a is 0 and b is 0;
(d) at least two, at least three, or at least four of R₁₁, R₁₂, R₁₃, and R₁₆ are H;
(e) if present, R₁₄ and R₁₅ are H;
(f) one of R₁ and R₂ is a substituted ring moiety;
(g) R₁ is a substituted ring moiety;
(h) R₂ is a substituted ring moiety;
(i) one of R₁ and R₂ is a substituted phenyl or substituted pyridyl;
and the other two of R₁, R₂ and R₃ are independently selected from hydrogen, halogen, and C₁₋₆ alkyl;
(j) wherein the substituent on said substituted phenyl or pyridyl is a para- or meta-substituent;
(k) wherein the substituent on said ring is of formula: X-Z or —X—W-Z, such as X—(C₁₋₆ alkylene)-Z, wherein X is selected from the group consisting of O, S, NR₂₁, —OCH₂—C≡C—, —NR₂₁—CO—, —CO—NR₂₁—, —NH—SO₂—, —SO₂—NH—, —NR₂₃—SO₂—, and —SO₂—NR₂₃; and Z is selected from (i) NR₂₁R₂₂ and pyridyl, piperidyl, and pyrrolidyl, optionally substituted;
(l) wherein a is 1 and b is 0; Y is N; one of R₁ and R₂ is phenyl para-substituted with X—W-Z, wherein X is O, NH, N(C₁₋₃ alkyl), NHCO, NHSO₂, or S; and W is C₂₋₅ alkylene;
(m) Z is piperidyl or pyrrolidyl, optionally substituted with methyl, CONH₂, or phenyl;
(n) R₁₁, R₁₂, R₁₃, and R₃ are each H;
(o) each of R₃, R₁₁, R₁₂, and R₁₃ is H, halo, methyl, or methoxy; or
(p) the ring moiety is substituted with —X—W-Z, —X-Z or W-Z; or
(q) combinations thereof.

Examples of most preferred compounds include: (S, S)-3-(4-(3-Piperidinylpropoxy)phenyl)octahydroindolizine; (R, R)-3-(4-(3-Piperidinylpropoxy)phenyl)octahydroindolizine; trans-3-(4-(3-Piperidinylpropoxy)phenyl)octahydroindolizine; anti-2-(4-(3-Piperidinylpropoxy)phenyl)octahydroindolizine; syn-2-[4-(3-Piperidinylpropanoxy)phenyl]octahydroindolizine; 3-[4-(Piperidinylpropoxy)phenyl]hexahydro-1H-pyrrolizine; 5-[4-(4-Piperidinylbutoxy)phenyl]indolizine; trans-3-[4-(N-5-Piperidylpentylamino)phenyl]octahydroindolizine; 5-[4-(3-Piperidinylpropoxy)phenyl]octahydroindolizine; 5-[4-(4-Piperidinylpentanoxy)phenyl]octahydroindolizine; N-Methyl-N-[4-(trans-Octahydro-3-indolizinyl)phenyl]-3-piperidinylpropenamide; trans-3-[4-(N-3-Piperidylpropylamino)phenyl]octahydroindolizine; trans-3-[4-(3-Piperidinylmethylpropargyloxy)phenyl]octahydroindolizine; trans-3-[4-(N-5-Piperidylpentanamido)phenyl]octahydroindolizine; trans-3-{4-[2,2′-(N-Methylpyrrolidinyl)ethoxy]phenyl}octahydroindolizine; anti-2-[3-(3-Piperidinylpropyloxy)phenyl]octahydroindolizine; trans-3-[4-(N-4-Piperidylbutanamido)phenyl]octahydroindolizine; trans-3-[4-(N-Methyl-N-3-piperidylpropylamino)phenyl]octahydroindolizine; trans-3-[4-(3-Piperidylsulfonylamino)phenyl]octahydroindolizine; 5-[4-(2-Piperidinylethanoxy)phenyl]octahydroindolizine; trans-3-{4-[2,2′-(N-Methylpiperidinyl)ethoxy]phenyl}octahydroindolizine; tran-3-[4-(4-Methylaminophenylthio)phenyl]octahydroindolizine; trans-3-[4-(N-Methyl-N-5-piperidylpentylamino)phenyl]octahydroindolizine; 3-[4-(2-Piperidin-1-yl-ethoxy)-phenyl]-octahydro-indolizine; Dimethyl-{3-[4-(octahydro-indolizin-3-yl)-phenoxy]-propyl}-amine; trans-3-[4-(N-3-Piperidinylpropanamido)phenyl]octahydroindolizine; trans-3-{4-[(2-Piperidylethyl)sulfonyl]amidophenyl}octahydro indolizine; trans-3-{4-[(2-Piperidylethyl)sulfonyl-N-methylamino]phenyl}octahydroindolizine; and tran-3-[4-(4-Carboxylicphenylthio)phenyl]octahydroindolizine.

Examples of compounds of the invention include: trans-3-[4-((4-Amidoxime)phenylthio)phenyl]octahydroindolizine; trans-3-[4-(4-Methansulfonaminophenoxy)phenyl]octahydroindolizine; trans-3-{4-[2,2′-(N-Trifluoroethylpiperidinyl)ethoxy]phenyl}octahydroindolizine; trans-3-{4-[2,2′-(1-tert-Butylcarboxylatepiperidinyl)ethoxy]phenyl}-octahydroindolizine; trans-3-[4-(3-Piperidylsulfonyl-N-methylamino)phenyl]octahydroindolizine; trans-3-[4-(4-Aminophenylthio)phenyl]octahydroindolizine; trans-3-[4-(N-Methyl-N-5-piperidylpentanamido)phenyl]octahydroindolizine; Octahydro-3-[4-(4-pyridinylthio)phenyl]indolizine; trans-3-[4-(N-Phenyl-1-piperazinylmethyl)phenyl]octahydroindolizine; trans-3-[4-(4-Pyridinylethenyl)phenyl]octahydroindolizine; trans-3-{4-[2,2′-(N-Trifluoroacetylpiperidinyl)ethoxy]phenyl}octahydroindolizine; tran-3-[4-(3-(2-Dimethylaminoethyl)amino)phenyl]octahydroindolizine; trans-3-[4-(4-Pyridyloxy)phenyl]octahydroindolizine; trans-3-{4-[2,2′-(N-Amidinopiperidinyl)ethoxy]phenyl}octahydroindolizine; trans-3-[4-(4-Pyridylmethan-1-ol)phenyl]octahydroindolizine; trans-3-[4-(2,2′-piperidinylethoxy)phenyl]octahydroindolizine; 4-[4-(Octahydro-indolizin-3-yl)-phenoxy]-quinazoline; trans-3-[4-(N-Methylsulfonyl)piperidinylamino)phenyl]octahydroindolizine; trans-3-[4-(3-bis-Methansulfonaminobenzyloxy)phenyl]octahydroindolizine; 3-(4-Thiophen-2-yl-phenyl)-octahydro-indolizine; trans-3-[4-(N-Methylsulfonyl-4-aminopiperidine)phenyl]octahydroindolizine; 4-[4-(4-Pyridylthio)phenyl]octahydroquinolizine; trans-3-[4-(3-Methansulfonaminobenzyloxy)phenyl]octahydroindolizine; and

trans-3-[4-(4-Trifluromethoxyphenyl)phenyl]octahydroindolizine.

Further examples of compounds of the invention include: 3-Biphenyl-4-yl-octahydro-indolizine; trans-3-(4-Phenoxy-phenyl)-octahydro-indolizine; cis-3-(4-Phenoxy-phenyl)-octahydro-indolizine; Dimethyl-[5-(octahydro-indolizin-3-yl)-naphthalen-1-yl]-amine; [4-(Octahydro-indolizin-3-yl)-phenyl]-diphenyl-amine; 5-[4-(4-Pyridinylthio)phenyl]octahydroindolizine; 5-[4-(4-Nitrophenylthio)phenyl]octahydroindolizine; 3-[4-(Pyridin-3-yloxy)-phenyl]-octahydro-indolizine; 2-[4-(Octahydro-indolizin-3-yl)-phenoxy]-1H-benzoimidazole; 3-[4-(4-Nitro-phenylsulfanyl)-phenyl]-octahydro-indolizine; 3-[4-(Pyrimidin-2-ylsulfanyl)-phenyl]-octahydro-indolizine; 2-[4-(Octahydro-indolizin-3-yl)-phenylsulfanyl]-3H-quinazolin-4-one; 2-[4-(Octahydro-indolizin-3-yl)-phenoxy]-quinoline; 2-Methyl-8-[4-(octahydro-indolizin-3-yl)-phenoxy]-quinoline; 4-[4-(Octahydro-indolizin-3-yl)-phenylsulfanyl]-benzonitrile; 5-(4-(4-Aminophenylthio)phenyl)octahydroindolizine; 3-Methylamino-3-(4-bromophenyl)octahydroindolizine; trans-3-[4-(4-Methylene-1,3-thiazolidine-2,4-diimine)phenyl]octahydroindolizine; 4′-(Octahydro-indolizin-3-yl)-biphenyl-3-ylamine; 3-(4-Thiophen-3-yl-phenyl)-octahydro-indolizine; 2-[4-(Octahydro-indolizin-3-yl)-phenyl]-thiophene-3-carbaldehyde; 4′-(Octahydro-indolizin-3-yl)-biphenyl-4-carbaldehyde; 3-(4′-Fluoro-biphenyl-4-yl)-octahydro-indolizine; and trans-3-[4-(3-hydroxyiminomethylthienyl)phenyl]octahydroindolizine.

The invention also encompasses the following compounds:

trans-3-[4-(3-Methylsulfonylaminophenyl)phenyl]octahydroindolizine; anti-2-[2-(3-Piperidinylpropoxy)phenyl]octahydroindolizine; trans-3-[4-(4-Aminophenoxy)phenyl]octahydroindolizine; trans-3-(4-Aminophenyl)octahydroindolizine; trans-3-(4-(N,N-Dimethylamino)phenyl)octahydroindolizine; trans-3-(4-(Methylsulfonylamino)phenyl)octahydroindolizine; trans-3-(4-(bis-Methylsulfonylamino)phenyl)octahydroindolizine; trans-3-{4-[4-(N-(1,1-dimethylethoxycarbonyl)piperidinylamino]phenyl}octahydroindolizine; trans-3-[4-(4-Piperidinylamino)phenyl]octahydroindolizine; trans-3-[4-(N-Ethyl-N-4-N-methylsulfonylpiperidinylamino)phenyl]octahydroindolizine; N-[4-(trans-Octahydro-3-indolizinyl)phenyl]propenamide; N-Methyl-N-[4-(trans-Octahydro-3-indolizinyl)phenyl]propenamide; and trans-3-{4-[(2-Pyrrolidylethyl)sulfonylamino]phenyl}octahydroindolizine.

Additional compounds include: trans-3-{4-[(4-Chlorophenyl)methan-1-ol]phenyl}octahydroindolizine; trans-3-{4-[(4-Chlorobenzyl]phenyl}octahydroindolizine; [4-(Octahydro-indolizin-3-yl)-phenyl]-pyridin-3-ylmethyl-amine; [4-(Octahydro-indolizin-3-yl)-phenyl]-pyridin-2-ylmethyl-amine; [4-(Octahydro-indolizin-3-yl)-phenyl]-thiophen-3-ylmethyl-amine; Furan-2-ylmethyl-[4-(octahydro-indolizin-3-yl)-phenyl]-amine; [4-(Octahydro-indolizin-3-yl)-phenyl]-pyridin-4-ylmethyl-amine; Benzyl-[4-(octahydro-indolizin-3-yl)-phenyl]-amine; [4-(Octahydro-indolizin-3-yl)-phenyl]-(1-oxy-pyridin-4-ylmethyl)-amine; (1H-Imidazol-2-ylmethyl)-[4-(octahydro-indolizin-3-yl)-phenyl]-amine; Dibenzyl-[4-(octahydro-indolizin-3-yl)-phenyl]-amine; (R, R)-Octahydro-3-[4-(4-pyridinylthio)phenyl]indolizine; and (S, S)-Octahydro-3-[4-(4-pyridinylthio)phenyl]indolizine.

Embodiments of the invention include formulae I, II and III

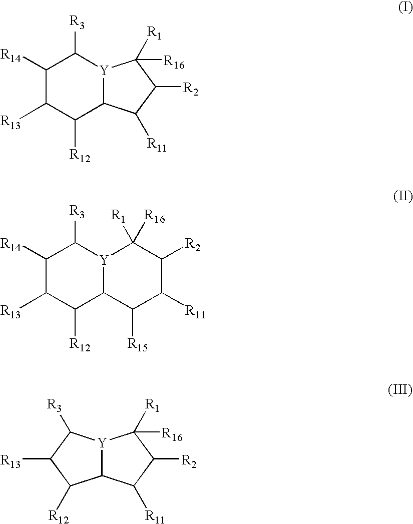

wherein all variables are as previously defined.

A more preferred embodiment of the present invention are compounds of Formulas I or III wherein:
R₁, R₂ and R₃ are independently selected from hydrogen, halogen, (C₁-C₆)alkyl, and a moiety of the formula:

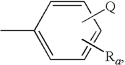

and, further, at least one of R₁, R₂, and R₃ in a compound of Formula I and at least one of R₁ and R₃ in a compound of Formula III is a moiety of said formula,
   wherein:
Q is a substituent of the formula:

—X—(CH₂)ₙ-Z
wherein X is selected from the group consisting of O, S, NH, NR₂₃, —OCH₂—C≡C—, —C≡C—CH₂O—, —NH—CO—, —CO—NH—, —NR₂₁—CO—, —CO—NR₂₁—,
—NH—SO₂—, —SO₂—NH—, —NR₂₁—SO₂—, and —SO₂—NR₂₁—, or X is a bond;
n is an integer from 0-5;
Z is selected from:
(ii) piperidyl, or pyrrolidyl,
(iii) an aryl group substituted by a heterocyclyl group, and an aryl group substituted by a heterocyclyl-alkyl group,
   wherein the heterocyclyl group in (ii) or (iii) may be substituted with one to four substituents independently selected from the group consisting of halo, hydroxy, (C₁-C₆)alkyl, (C₁-C₆)alkoxy, cyclohexyl, cyclohexenyl, aryl, substituted aryl, arylalkyl, trihalo(C₁-C₆)alkyl, nitro, SCH₃, NR₂₁R₂₂, amido, amidino, amino(C₁-C₆)alkyl, acetylene, CHR₂₃R₂₄, COR₂₅, acetyl, NHCOCH₃, heterocyclyl, heterocyclyl-alkyl, substituted heterocyclyl, substituted heterocyclyl-alkyl, cyano, NHSO₂CH₃, carboxy, (C₁-C₆)alkoxycarbonyl, amidoxime, trihalo(C₁-C₆)alkoxy, oxo, hydroxyiminomethyl, (C₁-C₆)alkylcarboxy, carboxy(C₁-C₆)alkyl, trihaloacetyl, and methylsulfonyl;
Rₐ is 1-4 substituents independently selected from hydrogen, halogen, (C₁-C₆)alkyl and (C₁-C₆)alkoxy;
R₁₁, R₁₂, R₁₄ and R₁₅ are independently selected from hydrogen, halogen, (C₁-C₆)alkyl and (C₁-C₆)alkoxy;
   R₁₃ is selected from hydrogen, oxo, and phenyl;
   R₁₆ is selected from hydrogen, cyano, (C₁-C₆)alkyl, and (C₁-C₆)alkylamino;
R₂₁, R₂₂, R₂₃, R₂₄ and R₂₅ are independently selected from hydrogen, halogen, (C₁-C₆)alkyl, (C₁-C₆)alkoxy, hydroxy, aryl, substituted aryl, (C₁-C₆)alkylamino, di(C₁-C₆)alkylamino, COR₂₆, SO₂R₂₆, and methylsulfonyl;
   R₂₆ is selected from hydrogen, heterocyclyl-alkyl, (C₁-C₆)alkyl, and (C₂-C₆)alkenyl.

Compounds useful as intermediates include the following: N1-[3-(Octahydro-indolizin-3-yl)-phenyl]-propane-1,3-diamine; [3-(Octahydro-indolizin-3-yl)-phenyl]-carbamic acid tert-butyl ester; 5-(4-Hydroxyphenyl)octahydroindolizine; anti-2-(2-Methoxyphenyl)octahydroindolizin; trans-3-(4-trimethylsilylacetylenephenyl)octahydroindolizine; trans-3-(4-acetylenephenyl)octahydroindolizine; trans-3-(4-Aminophenyl)octahydroindolizine; trans-3-(4-hydroxyphenyl)octahydroindolizine 5-[4-(4-Chlorobutanoxy)phenyl]octahydroindolizine anti-2-(4-Methoxyphenyl)octahydroindolizine syn-2-(4-Methoxyphenyl)octahydroindolizine 3-(4-Phenoxy-phenyl)-octahydro-indolizine; and cis-3-(4-Methoxy-phenyl)-octahydro-indolizine.

For use in medicine, the salts of the compounds of this invention refer to non-toxic “pharmaceutically acceptable salts.” Other salts may, however, be useful in the preption of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth salts, e.g., calcium or magnesium salts; and salts formed with suitable organic ligands, e.g., quaternary ammonium salts. Thus, representative pharmaceutically acceptable salts include the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preption of suitable prodrug derivatives are described, for example, in “Design of Prodrugs”, ed. H. Bundgaard, Elsevier, 1985.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

### C. SYNTHESIS

| | |
|---|---|
| DMAP = | dimethylaminopyridine |
| DMF = | dimethylformamide |
| DMSO = | dimethylsulfoxide |
| Et₃N = | triethylamine |
| EtOH = | ethanol |
| MeOH = | methanol |
| r.t. = | room temperature |
| TEA = | triethylamine |

The compounds of the present invention can be prepared according to the general Schemes 1 through 8. Thus the procedures of Schemes 1, 2 and 3 may be used to prepare indolizines as previously described in; Carmosin, R. J.; Carson, J. R. “Octahydroindolizine Compounds Useful as Analgesics”, U.S. Pat. No. 4,582,836, 1986; Carmosin, R. J.; Carson, J. R. “3-Diphenyl Substituted Octahydroindolizine Analgesic Compounds”, U.S. Pat. No. 4,683,239, 1987; Carmosin, R. J.; Carson, J. R. “5-Substituted Octahydroindolizine Analgesics Compounds and 7-Keto Intermediates”, U.S. Pat. No. 4,689,329, 1987, and Carson, J. R.; Carmosin, R. J.; Vaught, J. L.; Gardocki, J. F.; Costanzo, M. J.; Raffa, R. B.; Almond, H. R. J. Med. Chem. 1992, 35, 2855-2863. The procedure of Scheme 4 may used to prepare octahydroquinolizines as previously described in; Carmosin, R. J.; Carson, J. R. “4-Substituted Octahydroquinolizine Analgesic Compounds and Octahydroquinolizinium Intermediates”, U.S. Pat. No. 4,716,172, 1987. The procedure of Scheme 5 may be used to prepare related indolizines and relies on modifications to the Chichibabin indolizine synthesis (see for example, “Heterocyclic Chemistry”, 3^{rd}. Edition, Gilchrist, T. L., Longman, 1992). The procedures of Schemes 6 and 8 may be used to prepare pyrrolizines as previously described in; Carmosin, R. J.; Carson, J. R. “Hexahydropyrrolizines Compounds Useful as Analgesics”, U.S. Pat. No. 4,800,207, 1989. The methodology depicted in Scheme 1 for 3-aryl-substituted indolizines may readily be applied to the synthesis of 3-heteroaryl indolizines as shown in Scheme 7. Depending on the nature of the substituent Q′ the appropriate route can be selected.

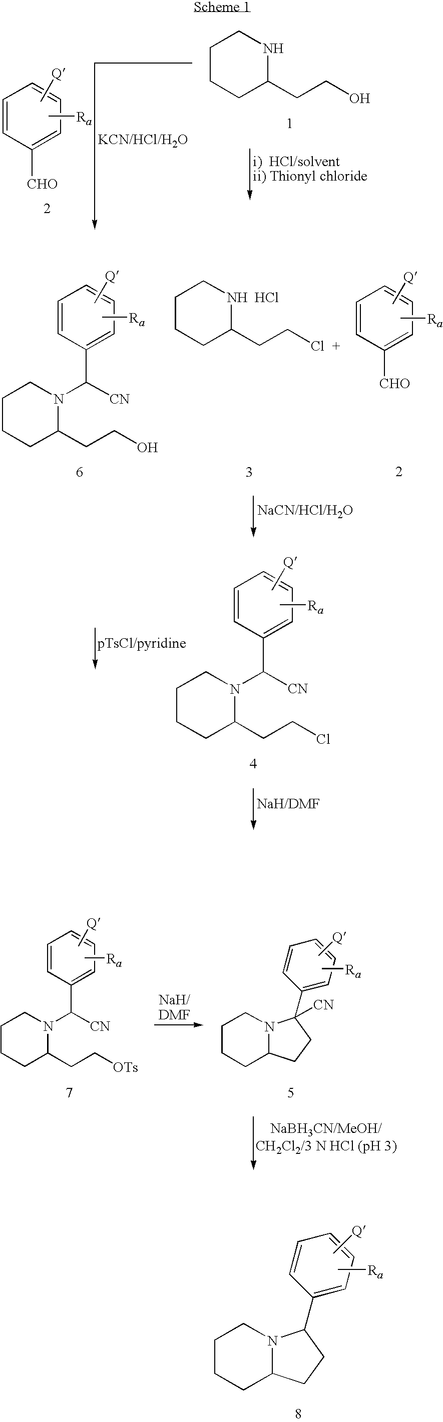

In accordance with Scheme 1, 2-piperidine ethanol is first treated with an inorganic acid such as HCl, and the like, to form the salt and then treated with thionyl chloride to produce the chloride 3. This is then reacted with the substituted benzaldehyde 2, where Q′ is a halogen, nitro, methoxy, benzlyoxy or similar group which can be easily modified or displaced for further functionalization, in the presence of an alkali metal cyanide salt such as sodium or potassium cyanide, in an aqueous solution at 0° to 50° C., preferably ambient temperature, in the presence of a strong acid such as HCl, to produce the chloroethyl-piperidineacetonitrile compound 4. Compound 4 is then cyclized by the action of a strong base such as sodium hydride, potassium hydride and the like in a polar aprotic solvent such as N,N-dimethylformamide, terahydrofuran or DMSO at room temperature to produce the cyano-octahydroindolizine 5. This compound is then reduced by a catalytic or hydride reducing agent such as sodium cyanoborohydride in methanol and dichloromethane after adjustment of the pH to about 3, or lithium aluminum hydride in dry diethylether or tetrahydrofuran, to produce the octahydroindolizine 8. The Q′ moiety can then be modified or displaced to further functionalize the compound as described below.

Alternatively, the octahydroindolizine can be produced from 2-piperidine ethanol by direct reaction with the substituted benzaldehyde in the presence of a cyanide salt to produce the hydroxyethyl-piperidine acetonitrile 6. This compound is then reacted with p-toluene-sulfonyl chloride in a solvent such as pyridine or triethylamine/methylene chloride to produce the piperidine ethylsulfonate 7. This is then cyclized with sodium hydride as described above to provide the cyano-octahydroindolizine 5.

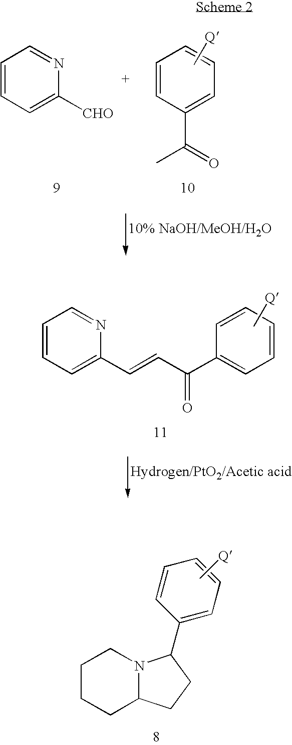

Scheme 2 illustrates an alternative method for the synthesis of the octahydroindolizine. In this method, described in U.S. Pat. No. 4,582,836, the pyridine-2-carboxaldehyde 9 undergoes a Claisen-Schmidt condensation with the substituted acetophenone 10 wherein Q′ is as described above, in the presence of an alkali metal hydroxide such as 10% NaOH, in a lower alkanol solvent at a temperature of −30° C. to +50° C., preferably about 10° C. to produce compound 11. Catalytic hydrogenation and cyclization of compound 11 with hydrogen gas in the presence of a catalyst such as palladium on carbon in the presence of an alkanoic acid or a lower alkanol such as acetic acid and ethanol, respectively, yields the octahydroindolizine 8.

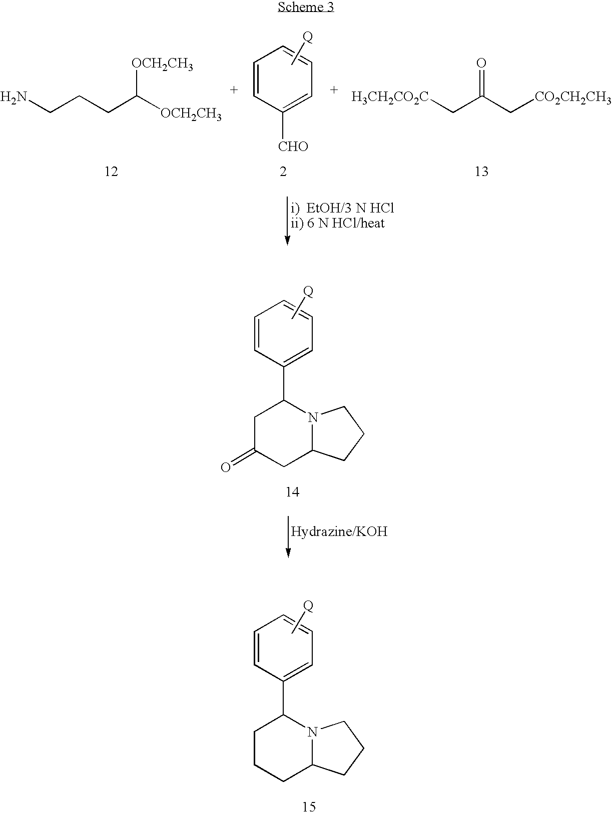

Scheme 3 illustrates the method for the synthesis of the 5-substituted octahydroindolizine compounds according to the method disclosed in U.S. Pat. No. 4,689,329. The benzaldehyde 2 is condensed with aminobutyraldehyde diethyl acetal 12 and diethyl acetone dicarboxylate 13 in an aqueous mineral acid at room temperature for several days to give a keto-diester which is not isolated. Heating with aqueous mineral acid gives the ketone 14. The ketone functionality is then reduced by treating with ahydrous hydrazine, in the presence of an alkali metal hydroxide such as KOH, at an elevated temperature gives the 5-substituted octahydroindozoline 15.

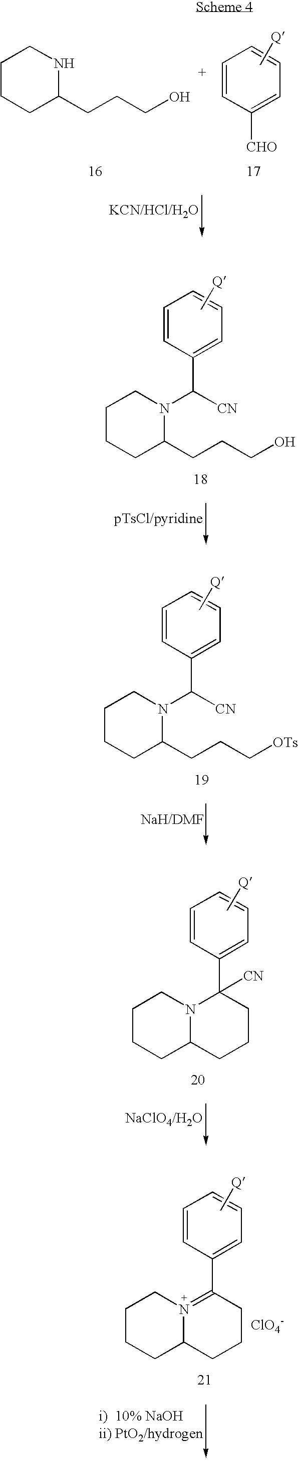

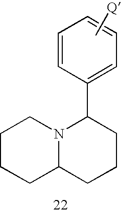

Scheme 4 illustrates the preparation of the 4-substituted octahydroquinolizine compounds according to the methods of U.S. Pat. No. 4,716,172. Analogous to the method shown in Scheme I, the benzaldehyde 17 is added to an aqueous solution of 3-(2-piperidine)-1-propanol HCl 16 in the presence of a cyanide salt to produce a hydroxypropyl piperidine acetonitrile 18. This compound is then reacted with p-toluene sulfonyl chloride to produce the piperidine propylysulfonate 19. Cyclization with a strong base such as sodium hydride yields the cyano quinolizine 20. Treatment with sodium perchlorate in water or other strong acid produces the octahydroquinolizium salt 21. The octahydroquinolizium salt is then reduced by a catalytic or hydride reducing agent to produce the final compound.

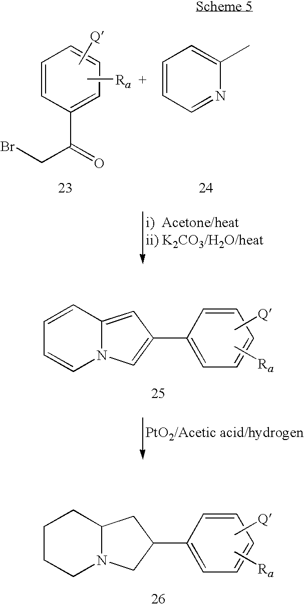

Scheme 5 demonstrates the preparation of the 2-substituted octahydroindolizines. A solution of the substituted 2-bromo acetophenone and 2-picoline in an organic solvent such as a lower alkyl ketone like acetone is heated to reflux. The solvent is then evaporated to form the quaternary salt. The salt is redissolved in hot water and treated with a base such as K₂CO₃, Na₂CO₃, KOH, DBU, TEA and the like to provide the 2-substituted indolizine 25. This compound is then reduced by catalytic hydrogenation in the presence of platinum(IV) oxide or the like to provide the octahydroindolizine 26. The reduction is accomplished at much lower temperatures and pressures than a reduction of 2-phenylindolizines reported by Zaporozhets et al (Zaporozhets, O. B.; Ryashentseva, M. A.; Polosin, V. M.; Poponova, R. V. Russ. Chem. Bull. 1993, 42(7), 1209-1210).

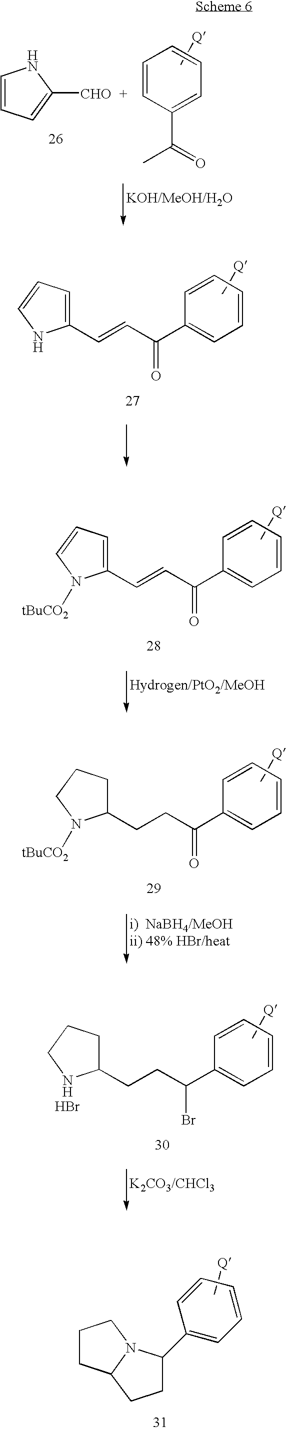

Scheme 6 depicts the preparation of pyrrolizines as previously described in U.S. Pat. No. 4,800,207. In this method, pyrrole-2-carboxaldehyde 26 is condensed with the benzaldehyde under Claisen-Schmidt conditions, for instance, in methanol and water in the presence of an alkali metal hydroxide to obtain compound 27. This is then reacted with di-tert-butyldicarbonate to afford the t-boc protected compound 28. This compound is then catalytically hydrogenated to produce the pyrrolidine-ketone 29. The pyrrolidine ketone is reduced with a hydride reducing agent and brominated with hydrogen bromide to produce the N-deprotected pyrrolidine hydrobromide 30. In the last step, the pyrrolidine compound 30 is cyclized to a hexahydropyrrolizine compound 31 by conversion of the hydrobromide salt to the free base and subsequent cyclization of the free base carried out by the action of a mild base such as potassium carbonate in a polar solvent such as water.

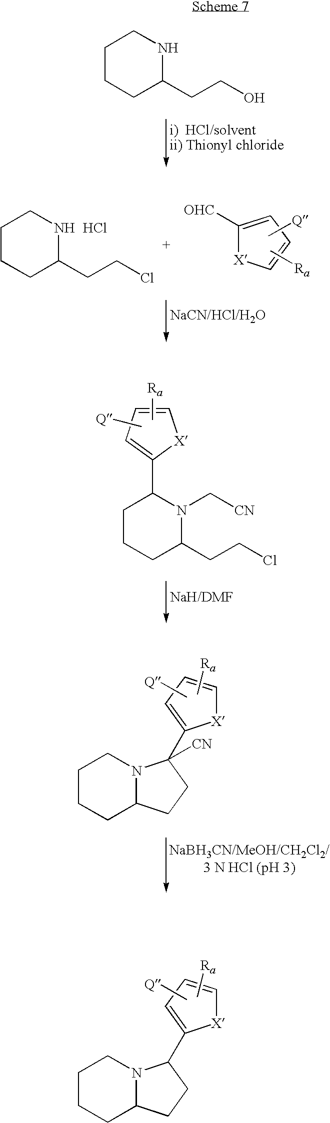

Scheme 7 illustrates the method of synthesis for those compounds where the moiety A is a heterocycle or heteroaryl. This method follows the procedure described in Scheme 1, substituting the appropriate heterocycle- or heteroaryl-carboxaldehyde for the benzaldehyde depicted in Scheme I. Q″ is a halogen, preferably Br, and X′ is O or S.

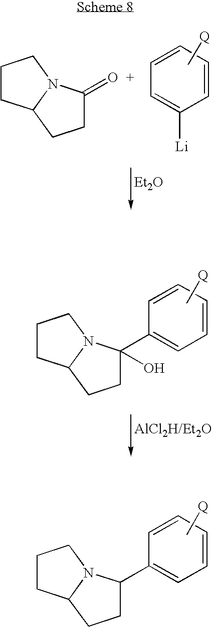

Scheme 8 depicts an alternative method for the preparation of the hexahydro-pyrrolizines of the invention.

Examples of further transformations of the substituent Q are depicted in Schemes 9 through 11. These transformations are well known functional group manipulations (see for example “Comprehensive Organic Transformations”, 2^{nd} Edition, Larock, R. C., Wiley-VCH, 1999 and “Comprehensive Organic Synthesis”, 1^{st} Edition, Trost, B. M., Elsevier Science Ltd., and references cited therein). In the schemes the various heterocyclic cores are represented by T. Thus in schemes 9 through 11, T represents the following:

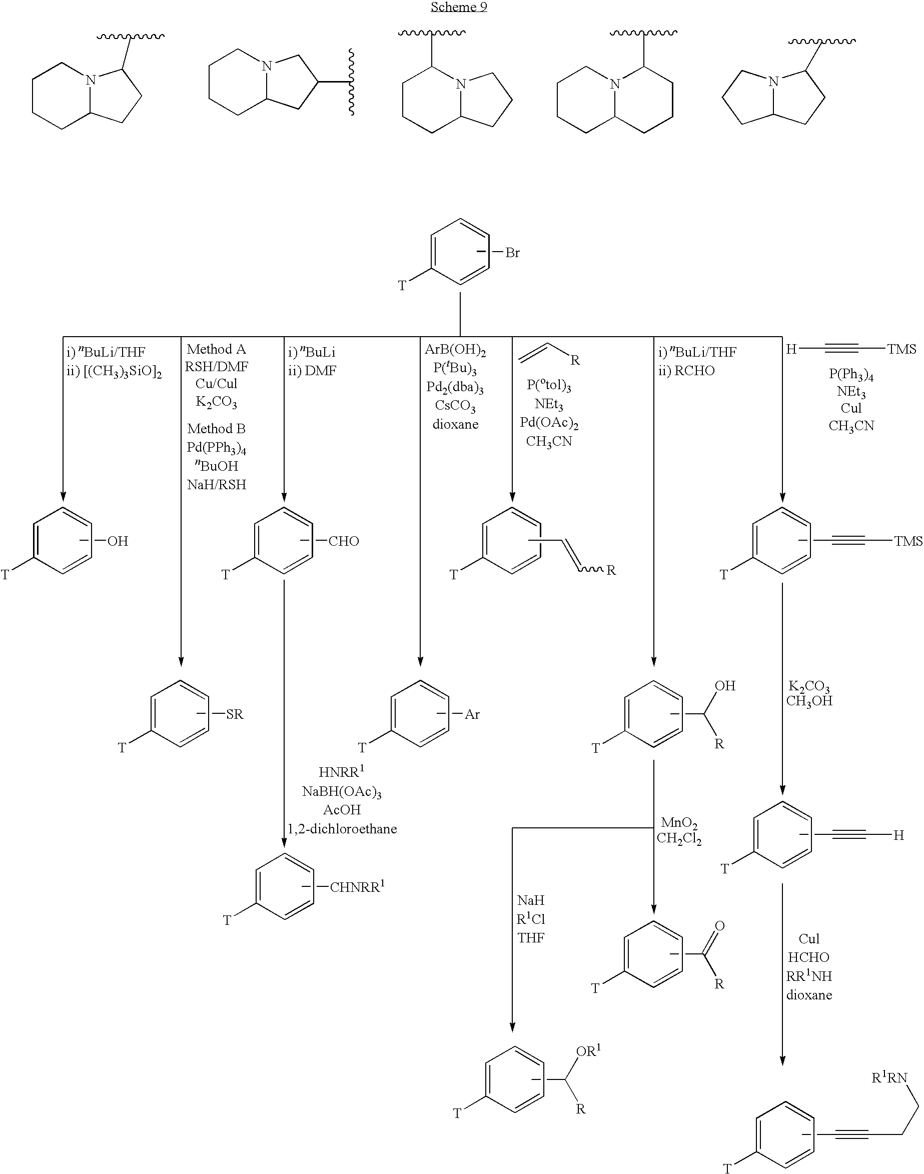

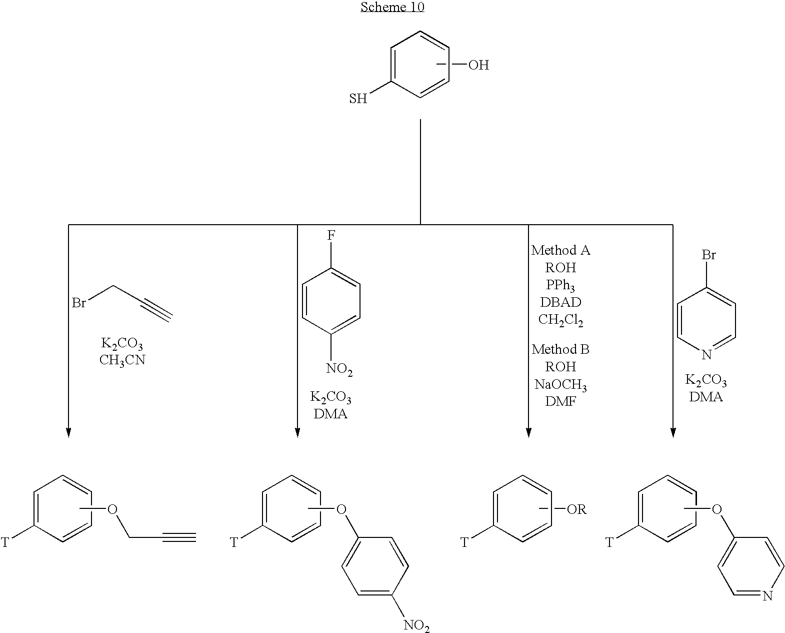

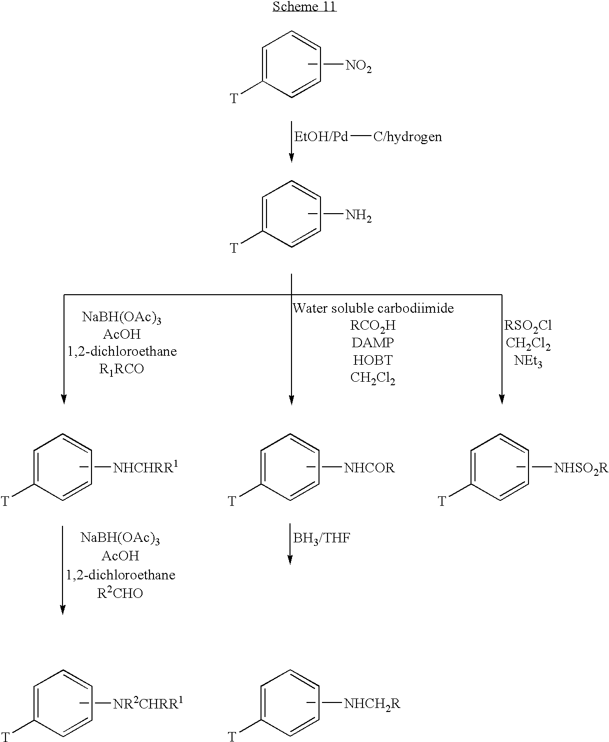

The invention also provides a process for the preparation of an octohydroindolizine of structural formula

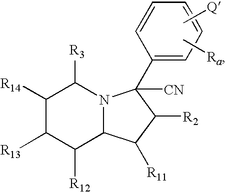

wherein:
R₂ and R₃ are independently selected from hydrogen, halogen, and (C₁-C₆)alkyl;
Q′ is 1-4 substituents independently selected from: halogen, nitro, methoxy, and benzyloxy;
Rₐ is 1-5 substituents independently selected from hydrogen, halogen, (C₁-C₆)alkyl and (C₁-C₆)alkoxy;
R₁₁, R₁₂, and R₁₄ are independently selected from hydrogen, halogen, (C₁-C₆)alkyl and (C₁-C₆)alkoxy;
and R₁₃ is selected from hydrogen, oxo, and phenyl;
   that comprises the step of reducing a cyano-octahydroindolizine of structural formula

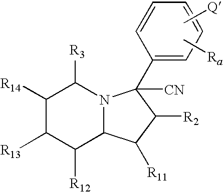

with NaBH₃CN to form the octahydroindolizine.
According to one aspect of this method, the cyano-octahydroindolizine is reduced by NaBH₃CN in methanol and dichloromethane after adjustment of the pH to 1-6, preferably adjustment to about 3.

The invention also provides a process for the preparation of an octohydroindolizine of structural formula

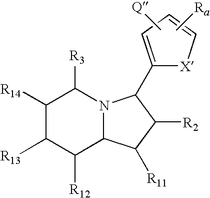

wherein:
R₂ and R₃ are independently selected from hydrogen, halogen, and (C₁-C₆)alkyl;
X′ is O or S;
Q″ is 0-2 independently selected halogens, preferably Br;
Rₐ is 1-3 substituents independently selected from hydrogen, halogen, (C₁-C₆)alkyl and (C₁-C₆)alkoxy;
R₁₁, R₁₂, and R₁₄ are independently selected from hydrogen, halogen, (C₁-C₆)alkyl and (C₁-C₆)alkoxy;
and R₁₃ is selected from hydrogen, oxo, and phenyl;
   wherein said method comprises the step of reducing a cyano-octahydroindolizine of structural formula

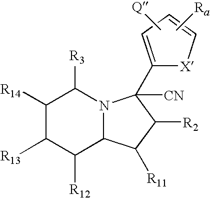

with NaBH₃CN to form the octahydroindolizine. In one aspect of this method, the cyano-octahydroindolizine is reduced by NaBH₃CN in methanol and dichloromethane after adjustment of the pH to between 1 and 6, preferably adjustment to about 3.

The invention also provides a process for the preparation of an octahydroindolizine of structural formula

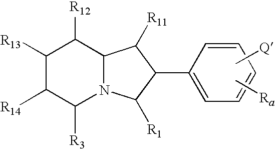

wherein:
R₁ is hydrogen or (C₁-C₆)alkyl;
R₃ is selected from hydrogen, halogen, and (C₁-C₆)alkyl;
Q′ is 0-4 substituents independently selected from: halogen, nitro, methoxy, and benzyloxy;
Rₐ is 1-5 substituents independently selected from hydrogen, halogen, (C₁-C₆)alkyl and (C₁-C₆)alkoxy;
R₁₁ is hydrogen or (C₁-C₆)alkyl;
R₁₂, and R₁₄ are independently selected from hydrogen, halogen, (C₁-C₆)alkyl and (C₁-C₆)alkoxy;
and R₁₃ is selected from hydrogen, oxo, and phenyl; wherein said method comprises the step of reducing an indolizine of structural formula

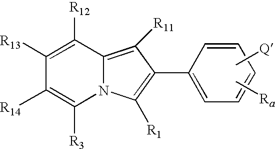

by catalytic hydrogenation at room temperature under 30 to 100 psi hydrogen, preferably 50-60 psi, in the presence of platinum(IV) oxide.

### D. FORMULATION, ADMINISTRATION, AND THERAPY

The disclosed compounds, alone or in combination (with, for example, a histamine H₁ receptor antagonist), are useful for treating or preventing neurologic disorders including sleep/wake and arousal/vigilance disorders (e.g. insomnia and jet lag), attention deficit hyperactivity disorders (ADHD), learning and memory disorders, cognitive dysfunction, migraine, neurogenic inflammation, dementia, mild cognitive impairment (pre-dementia), Alzheimer's disease, epilepsy, narcolepsy, eating disorders, obesity, motion sickness, vertigo, schizophrenia, substance abuse, bipolar disorders, manic disorders and depression, as well as other histamine H₃ receptor mediated disorders such as upper airway allergic response, asthma, itch, nasal congestion and allergic rhinitis in a subject in need thereof.

1. Formulation and Administration

The compounds or compositions of the invention may be formulated and administered to a subject by any conventional route of administration, including, but not limited to, intravenous, oral, subcutaneous, intramuscular, intradermal and parenteral administration. The quantity of the compound which is effective for treating each condition may vary, and can be determined by one of ordinary skill in the art.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth salts, e.g., calcium or magnesium salts; and salts formed with suitable organic ligands, e.g., quaternary ammonium salts. Thus, representative pharmaceutically acceptable salts include the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preption of suitable prodrug derivatives are described, for example, in “Design of Prodrugs”, ed. H. Bundgaard, Elsevier, 1985. In addition to salts, the invention provides the esters, amides, and other protected or derivatized forms of the described compounds.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

The present invention also provides pharmaceutical compositions comprising one or more compounds of this invention in association with a pharmaceutically acceptable carrier and optionally additional pharmaceutical agents such as H₁ antagonists or SSRIs. Preferably these compositions are in unit dosage forms such as pills, tablets, caplets, capsules (each including immediate release, timed release and sustained release formulations), powders, granules, sterile parenteral solutions or suspensions (including syrups and emulsions), metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 5 to about 1000 mg of the active ingredient of the present invention. Examples include 5 mg, 7 mg, 10 mg, 15 mg, 20 mg, 35 mg, 50 mg, 75 mg, 100 mg, 120 mg, 150 mg, and so on. The tablets or pills of the disclosed compositions can be coated or otherwise compounded to provide a dosage form affording the advantage of prolonged action. For example, the tablet or pill can comprise an inner dosage and an outer dosage component, the latter being in the form of an envelope over the former. The two components can be septed by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the compounds and compositions of the present invention may be incorporated for administration orally or by injection include, aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-d-tartaric acid and/or (+)-di-p-toluoyl-l-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The compound of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine or phophatidylcholines.

Compounds of the present invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamidephenol, polyhydroxyethylaspartamidephenol, or polyethyleneoxidepolylysine substituted with palmitoyl residue. Furthermore, the compounds of the present invention may be coupled to a class of biodegradable polymers useful in achieving controlled release of a drug, for example, polylactic acid, polyepsilon caprolactone, polyhydroxy butyric acid, polyoesters, polyacetals, polydihydropyrans, polycyanoacrylates and cross-linked or amphipathic block copolymers of hydrogels.

The daily dosage of the products may be varied over a wide range from 1 to 1,000 mg per adult human per day. For oral administration, the compositions are preferably provided in the form of tablets containing 1.0, 5.0, 10.0, 15.0, 25.0, 50.0, 100, 250 and 500 milligrams of the active ingredient for the symptomatic adjustment of the dosage to the subject to be treated. An effective amount of the drug is ordinarily supplied at a dosage level of from about 0.01 mg/kg to about 20 mg/kg of body weight per day. Preferably, the range is from about 0.02 mg/kg to about 10 mg/kg of body weight per day, and especially from about 0.05 mg/kg to about 10 mg/kg of body weight per day. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

2. Combination Therapy

The disclosed compounds are useful in combination with other therapeutic agents, including H, receptor antagonists, H₂ receptor antagonists, and neurotransmitter modulators such as SSRIs and non-selective serotonin re-uptake inhibitors (NSSRIs).

Methods are known in the art for determining effective doses for therapeutic and prophylactic purposes for the disclosed pharmaceutical compositions or the disclosed drug combinations, whether or not formulated in the same composition. For therapeutic purposes, the term “jointly effective amount” as used herein, means that amount of each active compound or pharmaceutical agent, alone or in combination, that elicits the biological or medicinal response in a tissue system, animal or human that is being sought by a researcher, veterinarian, medical doctor or other clinician, which includes alleviation of the symptoms of the disease or disorder being treated. For prophylactic purposes (i.e., inhibiting the onset or progression of a disorder), the term “jointly effective amount” refers to that amount of each active compound or pharmaceutical agent, alone or in combination, that inhibits in a subject the onset or progression of a disorder as being sought by a researcher, veterinarian, medical doctor or other clinician, the delaying of which disorder is mediated, at least in part, by the modulation of one or more histamine receptors. Thus, the present invention provides combinations of two or more drugs wherein, for example, (a) each drug is administered in an independently therapeutically or prophylactically effective amount; (b) at least one drug in the combination is administered in an amount that is sub-therapeutic or sub-prophylactic if administered alone, but is therapeutic or prophylactic when administered in combination with the second or additional drugs according to the invention; or (c) both drugs are administered in an amount that is sub-therapeutic or sub-prophylactic if administered alone, but are therapeutic or prophylactic when administered together. Combinations of three or more drugs are analogously possible. Methods of combination therapy include co-administration of a single formulation containing all active agents; essentially contemporaneous administration of more than one formulation; and administration of two or more active agents separately formulated.

During any of the processes for preparation of the compounds of the present invention, it may be necessary and/or desirable to protect sensitive or reactive groups on any of the molecules concerned. This may be achieved by means of conventional protecting groups, such as those described in “Protective Groups in Organic Chemistry”, ed. J. F. W. McOmie, Plenum Press, 1973; and T. W. Greene & P. G. M. Wuts, “Protective Groups in Organic Synthesis”, John Wiley & Sons, 1991. The protecting groups may be removed at a convenient subsequent stage using methods known from the art.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms may include suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

The compound of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine or phophatidylcholines.

Compounds of the present invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamidephenol, polyhydroxyethylaspartamidephenol, or polyethyl-eneoxidepolylysine substituted with palmitoyl residue. Furthermore, the compounds of the present invention may be coupled to a class of biodegradable polymers useful in achieving controlled release of a drug, for example, polylactic acid, polyepsilon caprolactone, polyhydroxy butyric acid, polyoesters, polyacetals, polydihydropyrans, polycyanoacrylates and cross-linked or amphipathic block copolymers of hydrogels.

The daily dosage of the products may be varied over a wide range from 5 to 1,000 mg per adult human per day. For oral administration, the compositions are preferably provided in the form of tablets containing, 5.0, 10.0, 15.0, 25.0, 50.0, 100, 250 and 500 milligrams of the active ingredient for the symptomatic adjustment of the dosage to the patient to be treated. An effective amount of the drug is ordinarily supplied at a dosage level of from about 0.1 mg/kg to about 20 mg/kg of body weight per day. Preferably, the range is from about 0.2 mg/kg to about 10 mg/kg of body weight per day, and especially from about 0.5 mg/kg to about 10 mg/kg of body weight per day. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

### E. EXAMPLES

The following Examples are set forth to aid in the understanding of the invention, and are not intended and should not be construed to limit in any way the invention set forth in the claims.

Unless otherwise indicated, ¹H NMRs were run on a Bruker 400 MHz instrument (Bruker Analytik GmbH). Mass spectrometry was performed on a Hewlett Packard Series 1100 MSD (Hewlett-Packard GmbH).

### Example 1

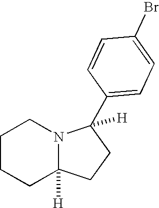

### trans-3-(4-bromophenyl)octahydroindolizine Kᵢ=325 nM

### Step A 2-(2-Chloroethyl)piperidine hydrochloride

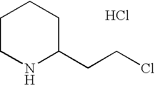

2-Piperidine ethanol (25 g) was treated with HCl in dioxane (4M, 48 mL) and excess solvent was evaporated. The residue was heated with thionyl chloride (34 mL) in chloroform under reflux temperature for 3 hours. The reaction mixture was then cooled to room temperature and the solvent removed in vacuo to afford the title compound as a yellow solid (30 g) which was used without further purification.

### Step B alpha-(4-Bromophenyl)-2-(2-chloroethyl)-1-piperdineacetonitrile

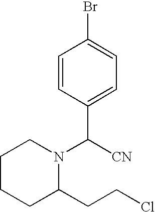

A mixture of the product of Step A (38.2 g) and 4-bromobenzaldehyde (38.4 g) in water (250 mL) was treated with sodium cyanide (11.2 g). The mixture was stirred at ambient temperature for 3 days. The mixture was extracted with diethylether (5×100 mL) and the combined organic extracts dried over sodium sulfate, filtered and concentrated to give a crude oil containing the title compound (71 g) which was used without further purification.

### Step C 3-Cyano-3-(4-bromophenyl)octahydroindolizine

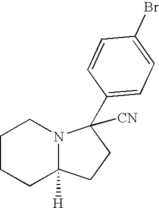

To sodium hydride (12.4 g, 60% dispersion in mineral oil) in 75 mL of N,N-dimethylformamide the product of Step B was added dissolved in 450 mL of N,N-dimethylformamide. After addition was complete the mixture was stirred overnight at ambient temperature. The mixture was diluted with water (500 mL) and then extracted with diethylether (10×100 mL). The combined organic extracts were dried over sodium sulfate, filtered and concentrated to afford a crude solid (60 g) that was used without further purification.

### Step D trans-3-(4-Bromophenyl)octahydroindolizine

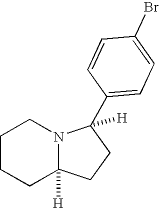

A solution of the product of Step C (75 g) in methanol (500 mL) and dichloromethane (360 mL) was treated with sodium cyanoborohydride (15.36 g), a trace of methyl orange and 3N HCl via an addition funnel to maintain the pH of the mixture at 3. The mixture was quenched by the addition of saturated sodium bicarbonate solution. The organic layer was separated and the aqueous layer extracted with dichloromethane (5×50 mL). The combined organic extracts were dried over sodium sulfate, filtered and concentrated. The orange oil was purified via silica gel chromatography (ethylacetate/hexanes) to give the title compound (51 g).

### Alternative Synthesis for Step D:

A suspension of the product of Step C (7 g) in dry diethylether or tetrahydrofuran (100 mL) was treated with lithium aluminum hydride (4 g) at room temperature for 15 hours. Addition of water, sodium hydroxide and water gave a precipitate. The precipitate was removed by filtration and the filtrate was evaporated in vacuo. The residue was purified via silica gel chromatography (ethylacetate/hexanes) to give the title compound (3 g).

The resolution of this material into the corresponding enantiomers was accomplished according to the procedures described in U.S. Pat. No. 4,683,239; Example 1.

The following compounds were prepared according to the procedure of Example 1. The reacting aldehydes and the resulting products (Example 2-21) are shown in Table 1. All the examples are isolated as racemates unless noted otherwise.

TABLE 1

| Example | Aldehyde | Product | Kᵢ (nM) |
|---|---|---|---|
| 2 | 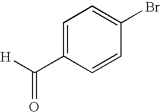 | 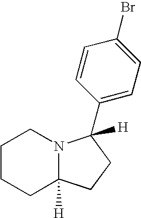 | 1000 |
| 3 | 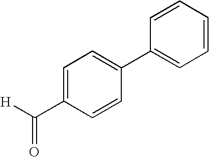 | 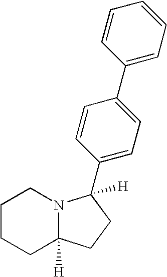 | 1000 |
| 7 | 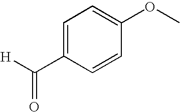 | 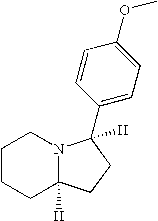 | 5000 |
| 8 | 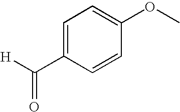 | 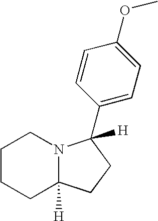 | 5000 |
| 11 | 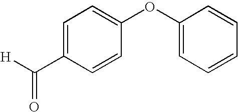 | 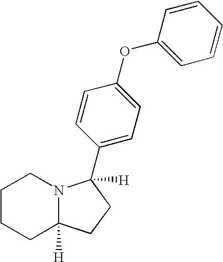 | 400 |
| 12 | 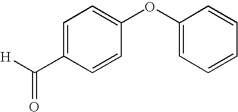 | 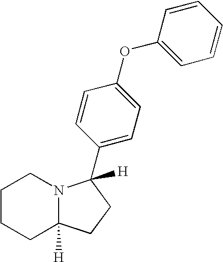 | 600 |
| 14 | 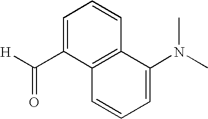 | 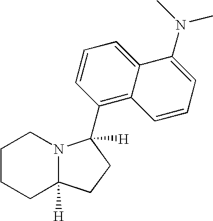 | 5000 |
| 15 | 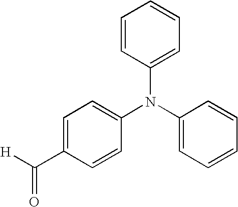 | 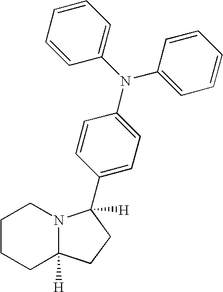 | 5000 |
| 16 | 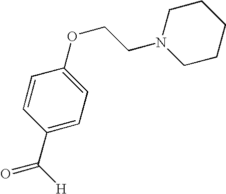 | 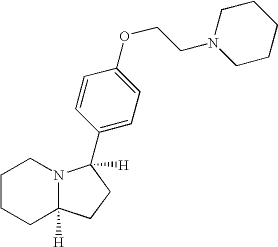 | 9 |
| 17 | 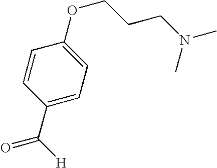 | 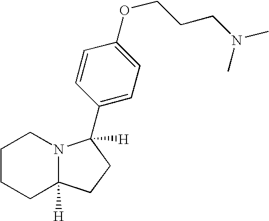 | 10 |

### Example 16

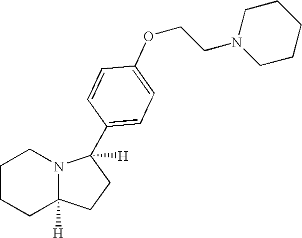

### 3-[4-(2-Piperidin-1-yl-ethoxy)-phenyl]-octahydro-indolizine Kᵢ=9.1 nM

¹H NMR (400 MHz, CDCl₃) δ 7.24 (d, J=8.5 Hz, 2H), 6.84 (d, J=8.5 Hz, 2H), 4.09 (t, J=6.2 Hz, 2H), 3.04 (t, J=8.0 Hz, 1H), 2.75 (m, 3H), 2.50 (bs, 4H), 2.02 (m, 2H), 1.77 (m, 4H), 1.59 (m, 6H), 1.44 (m, 4H), 1.26 (m, 2H).

### Example 17

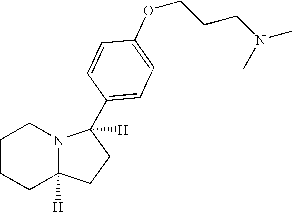

### Dimethyl-{3-[4-(octahydro-indolizin-3-yl)-phenoxy]-propyl}-amine Kᵢ=9.5 nM

¹H NMR (400 MHz, CDCl₃) δ 7.23 (d, J=8.5 Hz, 2H), 6.84 (d, J=8.5 Hz, 2H), 3.99 (t, J=6.4 Hz, 2H), 3.04 (t, J=8.0 Hz, 1H), 2.74 (d, J=10.2 Hz, 1H), 2.44 (t, J=7.4 Hz, 2H), 2.25 (s, 6H), 1.99 (m, 4H), 1.80 (m, 4H), 1.53 (m, 4H), 1.26 (m, 2H).

### Example 21

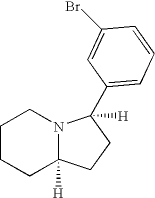

### trans-3-(3-Bromophenyl)octahydroindolizine m.p. 195-198° C.

This compound was prepared according to the procedure described in J. Med. Chem., 1992, 35, 2855; Scheme I.

The following compound was prepared in racemic form.

### Example 22

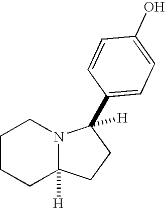

### trans-3-(4-hydroxyphenyl)octahydroindolizine

A solution of trans-3-(4-bromophenyl)octahydroindolizine (the product of Example 1, 1.0 g) in 20 mL of tetrahydrofuran was treated with n-butyllithium (2.17 mL, 2.0M in cyclohexane) at −78° C. The mixture was stirred at −78° C. for 30 minutes and then treated with bis(trimethylsilyl)peroxide (776 mg) at −78° C. and then allowed to warm to ambient temperature. The mixture was diluted with diethylether and washed with saturated ammonium chloride solution. The organic extracts were dried over sodium sulfate, filtered and concentrated in vacuo. The residue was purified via silica gel chromatography (ethylacetate/hexanes) to give the title compound (0.62 g). (c.f. Taddei, M.; Ricci, A. Synthesis, 1986, 633, for the application of bis(trimethylsilyl)peroxide). ¹H NMR (400 MHz, CDCl₃) δ 7.19 (d, J=8.6 Hz, 2H), 6.9 (d, J=8.6 Hz, 2H), 3.77 (dd, J=4.7, 4.7 Hz, 1H), 3.07 (t, J=8.3 Hz, 1H), 2.87-2.81 (m, 1H), 2.51-2.45 (m, 1H), 2.10-2.00 (m, 2H), 1.91-1.20 (m, 8H).

The following compounds were prepared according to Scheme 7. All the examples were isolated as racemates unless noted otherwise.

### Example 23

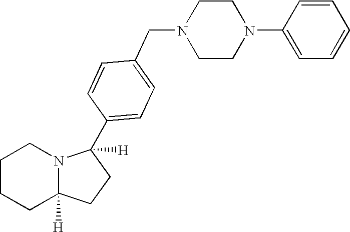

### trans-3-[4-(N-Phenyl-1-piperazinylmethyl)phenyl]octahydro indolizine Kᵢ=65 nM

### Step A trans-3-(4-Formylphenyl)octahydroindolizine

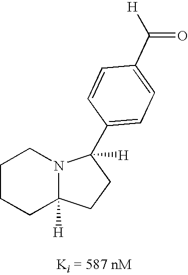

A solution of the product of Example 1 (500 mg) in tetrahydrofuran (8 mL) was cooled in a dry ice-acetone bath, and a solution of n-butyllithium in hexanes (1.6M, 1.2 mL) was added dropwise. After 1 hour, the resulting mixture was treated with N,N-dimethylformamide (0.65 mL), and allowed to warm to room temperature over 2 hours. Water (5 mL) was added and the organic phase was extracted with diethylether (1×20 mL). The combined organic phases were washed with brine (1×5 mL) and dried (magnesium sulfate). Removal of solvent in vacuo gave the title compound as a yellow oil (355 mg) which was used without further purification.

### Step B trans-3-[4-(N-phenyl-1-piperazinylmethyl)phenyl]octahydroindolizine

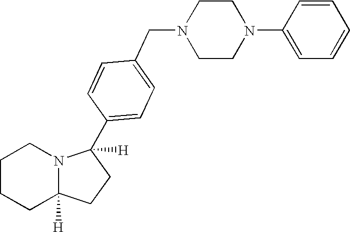

The product of Step A (50 mg) was treated with a solution prepared from N-phenylpiperazine (39 mg) and a solution of acetic acid in 1,2-dichloroethane (0.2M, 1.0 mL). After 5 min, sodium triacetoxyborohydride (65 mg) was added, and the resulting mixture was stirred vigorously for 2 hours. Saturated aqueous sodium bicarbonate (1 mL) was slowly added. The aqueous phase was extracted with dichloromethane (3×1 mL), and the combined organic phases were dried (magnesium sulfate), and evaporated. The residue was purified by silica gel chromatography (methanol/dichloromethane) to giving the title compound as a pale yellow glassy solid (40 mg).

¹H NMR (400 MHz, CDCl₃) δ 7.35-7.26 (m, 6H), 6.95 (d, J=7.8 Hz, 2H), 6.87 (t, J=7.3 Hz, 1H), 3.58 (s, 2H), 3.23 (t, J=4.9 Hz, 4H), 3.15 (t, J=8.1 Hz, 1H), 2.85-2.78 (m, 1H), 2.64 (t, J=4.9 Hz, 4H), 2.15-2.10 (m, 2H), 1.91-1.77 (m, 4H), 1.69-1.45 (m, 4H), 1.40-1.20 (m, 2H).

### Example 24

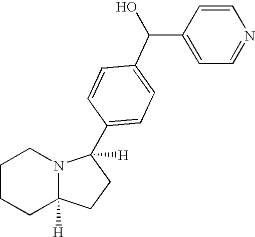

### trans-3-[4-(4-Pyridylmethan-1-ol)phenyl]octahydroindolizine Kᵢ=82 nM

A solution of the product of Example 1 (200 mg) in tetrahydrofuran (3 mL) was cooled in a dry ice-acetone bath, and a solution of n-butyllithium in hexanes (1.6M, 0.49 mL) was added dropwise. After 30 min, the resulting mixture was treated with a solution of 4-pyridinecarboxaldehyde (0.09 mL) in tetrahydrofuran (1 mL). The resulting mixture was allowed to warm to room temperature after 45 min. Water (1 mL) was added and the organic phase was extracted with diethylether (1×3 mL). The combined organic phases were concentrated in vacuo to give a reddish solid. Trituration of this solid with diethylether (1×1 mL) gave the title compound (94 mg).

¹H NMR (400 MHz, CDCl₃) δ 8.47 (d, J=6.0 Hz, 2H), 7.35-7.25 (m, 6H), 5.78 (s, 1H), 3.55 (br s, 1H), 3.13 (t, J=8.0 Hz, 1H), 2.77-2.72 (m, 1H), 2.12-2.00 (m, 2H), 1.87-1.74 (m, 4H), 1.61-1.17 (m, 6H).

The following compounds were prepared according to Scheme 1 and 8. All the examples were isolated as racemates unless noted otherwise.

### Example 25

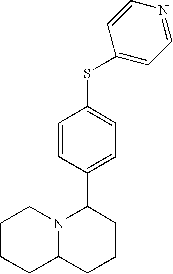

### 4-[4-(4-Pyridylthio)phenyl]octahydroquinolizine Kᵢ=305 nM

### Step A 3-(2-Piperidinyl)-1-propanol hydrobromide

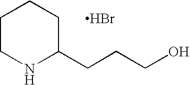

A solution of 3-(2-Pyridyl)-1-propanol (50 g) in aqueous HBr (1000 mL, 2.8%) was flushed with nitrogen and platinum(IV) oxide (4 g) was added. The reaction mixture was hydrogenated at 15-40 psi and room temperature for 4 hours. The reaction mixture was filtered and evaporated to give crude material (96 g) that was used without further purification.

### Step B 2-(3-Hydroxypropyl)-alpha-(4-bromophenyl)-1-piperidineacetonitrile

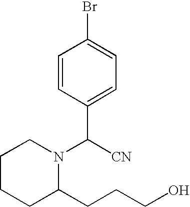

To a stirred suspension of the product of Step A (96 g) and 4-bromobenzaldehyde (71 g) in water (500 mL) was added at room temperature sodium cyanide (20 g). The reaction mixture was stirred for 24 hours at room temperature and was extracted with diethylether (3×350 mL). The combined organic layers were washed with aqueous 1N HCl (2×250 mL), saturated sodium hydrogencarbonate (2×250 mL), water (250 mL) and brine (250 mL). The organic layers were dried over magnesium sulfate and evaporated to give the crude material (92 g) which was used without further purification.

### Step C 2-[3-(4-Methylbenzensulfonyl)propyl]-alpha-(4-bromophenyl)-1-piperidineacetonitrile

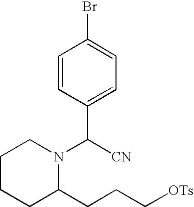

To a solution of the product of Step B (92 g) in pyridine (100 mL) was added in portions 4-toluenesulfonyl chloride (54 g) at 0° C. The reaction mixture was stirred for 3.5 hours at 0° C. and diethylether (1000 mL) and water (500 mL) were added. The organic layer was washed with aqueous 1N HCl (2×200 ml), water (200 mL) and brine (200 mL) and was dried over magnesium sulfate. The organic layer was evaporated to give the crude material (104 g) which was used without further purification.

### Step D 4-(4-Bromophenyl)-4-octahydroquinolizinecarbonitrile

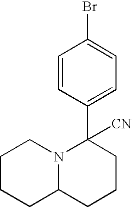

To a stirred mixture of sodium hydride (8 g, 60% suspension in paraffin oil) and N,N-dimethylformamide (100 mL) in a room temperature water bath was added a solution of the product of Step C (104 g) in N,N-dimethylformamide (400 mL). The reaction mixture was stirred for 24 hours at room temperature and water (1000 mL) was added. The aqueous layer was extracted with diethylether (4×250 mL) and the combined organic layers were washed with water (250 mL) and brine (250 mL). The organic layers were evaporated to give the crude material (25 g) which was used without further purification.

### Step E 4-(4-Bromophenyl)-4-octahydroquinolizine

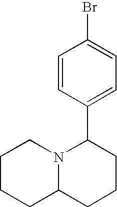

To a stirred solution of the product of Step D (1.6 g) in methanol (12 mL) and dichloromethane (12 mL) was added at room temperature sodium cyanoborohydride. The pH value of the reaction mixture was maintained between 3 and 1 by adding aqueous 3N HCl. After stirring at room temperature for 4 hours water (100 mL) was added and the organic solvent removed in vacuo. The aqueous layer was brought to a pH value of 9 by adding saturated sodium hydrogencarbonate and was extracted with diethylether (2×200 mL). The combined organic layers were washed with water, diethylether (2×75 mL) and brine (75 mL) and dried over magnesium sulfate. The organic layers were evaporated and the residue was purified via silica gel chromatography (ethylacetate/hexanes) to give the title compound (1 g).

### Step F 4-[4-(4-Pyridylthio)phenyl]octahydroquinolizine, bistrifluoroacetate

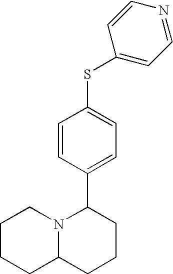

To sodium hydride (140 mg, 60% suspension in paraffin oil) was added at room temperature n-butanol followed by 4-mercaptopyridine (389 mg), the product of Step E (1 g) and tetrakis(triphenylphosphine)palladium(0). The reaction mixture was heated at reflux temperature for 8 hours and then allowed to cool to room temperature. Diethylether (500 mL) was added and the organic layer was washed with water (3×100 mL) and brine (100 mL). The organic layer was dried over magnesium sulfate and evaporated. The residue was purified via silica gel chromatography (hexanes/acetone and chloroform/methanol) followed by HPLC (RP18, acetonitrile/aqueous trifluoroacetic acid) to give the title compound (8 mg).

¹H NMR (400 MHz, methanol-d₄): δ=8.46 (d, J=6.9 Hz, 2H)), 7.72-7.84 (m, 4H), 7.53-7.56 (m, 2H), 4.29-4.35 (m, 1H), 3.28-3.36 (m, 1H), 3.06-3.11 (m, 1H), 2.81-2.89 (m, 1H), 1.56-2.23 (m, 12H).

The following compounds were prepared according to Schemes 3 and 9. Products were isolated as racemates unless noted otherwise.

### Example 26

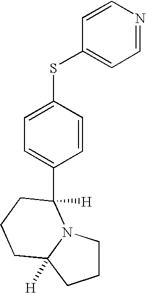

### 5-[4-(4-Pyridinylthio)phenyl]octahydroindolizine Kᵢ=35 nM

### Step A 5-(4-Bromophenyl)hexahydro-7(8H)-indolizinone

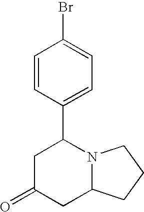

To a solution of 4-aminobutyraldehyde diethylacetal (0.084 mol of 90% technical grade, 13.3 g,) in absolute ethanol (75 mL) was added 3N HCl (28 mL), 4-bromobenaldhyde (13.9 g, 0.075 mol) and diethyl 1,3-acetonedicarboxylate (14.3 mL, 0.075 mol). The reaction mixture was stirred at room temperature for 3 days. Potassium carbonate (6.0 g, 0.0438 mol) and water (25 mL) was added and partitioned between diethylether (100 mL) and water. The diethylether layer was extracted with 6N HCl (125 mL). The aqueous acid solution was heated to 95° C. to remove residual diethylether and ethanol, then heated at reflux temperature overnight. After cooling to room temperature, 3N sodium hydroxide was added to make the solution basic. Extraction with diethylether, concentration, and chromatography on silica gel (ethylacetate/hexanes: 5/95) afforded the title compound (3.3 g, 15%).

### Step B 5-(4-Bromophenyl)octahydroindolizine

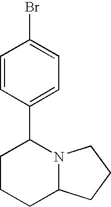

The mixture of 5-(4-bromophenyl)hexahydro-7(8H)-indolizinone (the product of Step A, 1.2 g, 4.08 mmol), hydrazine (0.128 mL, 4.08 mmol), potassium hydroxide (0.48 g, 8.57 mmol) in 2-hydroxyethyldiethylether was heated at 100 for 1 hour. The mixture was then distilled at 220-240° C. over 2 h. The distillate was partitioned between diethylether and water and the organic layer dried and concentrated. The residue was purified by column chromatography to afford the title compound (0.9 g, 79%).

### Step C 5-(4-(4-Pyridinylthio)phenyl)octahydroindolizine

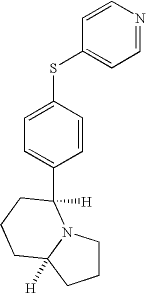

The mixture of 5-(4-bromophenyl)octahydroindolizine (53 mg, 0.188 mmol), 4-thiopyridine (21 mg, 0.188 mmol), copper (2.4 mg, 0.0376 mmol), copper iodide (I) (2.5 mg, 0.01316 mmol), and potassium carbonate (57 mg, 0.414 mmol) in N,N-dimethylformamide (2 mL) was heated at 140° C. for 2 days. Then N,N-dimethylformamide was evaporated. Preparative thin layer chromatography of the residue afforded the title compound (25 mg, 43%).

¹H NMR (400 MHz, CDCl₃) δ 8.26 (m, 2H), 7.40 (2d, J=8.3 Hz, 4H), 6.85 (m, 2H), 2.95 (dd, J=2.7, 10.7 Hz, 1H), 2.67 (td, J=8.6, 1.9 Hz, 1H), 2.00-1.15 (m, 12H).

### Example 27

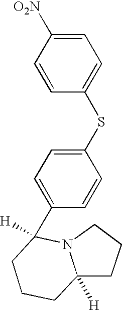

### 5-[4-(4-Nitrophenylthio)phenyl]octahydroindolizine Kᵢ=323 nM

The mixture of 5-(4-bromophenyl)octahydroindolizine (212 mg, 0.75 mmol, the product of Step B of Example 26), 4-nitrobenzenethiol (118 mg of 80% technical grade, 0.75 mmol), copper (9.6 mg, 0.15 mmol), copper iodide (I) (10 mg, 0.052 mmol) and potassium carbonate (229 mg, 1.65 mmol) in N,N-dimethylformamide (6 mL) was heated at 140° C. for 2 days. Then N,N-dimethylformamide was evaporated. Preparative thin layer chromatography of the residue afforded the title compound (210 mg, 79%).

The following compounds were prepared from Example 1 according to the procedure of Example 27 (Scheme 8). The starting materials and corresponding products are shown in Table 2. The products were isolated as racemates unless noted otherwise.

TABLE 2

| Example | Aryl | Product | Kᵢ (nM) |
|---|---|---|---|
| 28 | 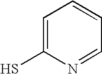 | 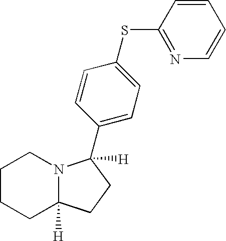 | 205 |
| 29 | 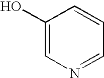 | 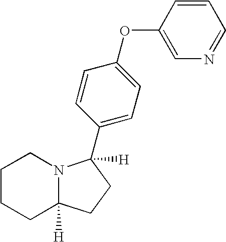 | 150 |
| 30 | 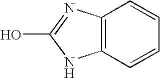 | 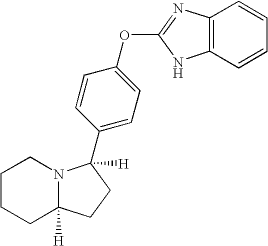 | 490 |
| 31 | 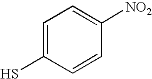 | 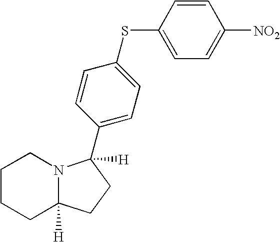 | 1000 |
| 32 | 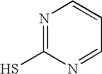 | 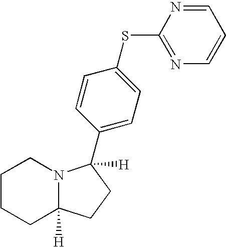 | 650 |
| 33 | 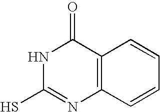 | 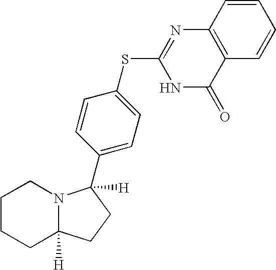 | 163 |
| 34 | 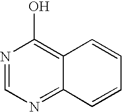 | 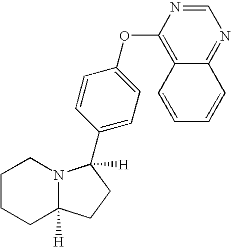 | 92 |
| 35 | 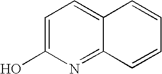 | 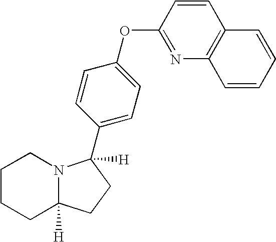 | 1000 |
| 36 | 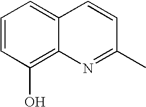 | 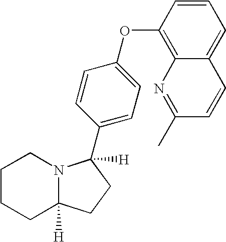 | 1000 |
| 38 | 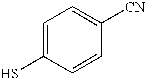 | 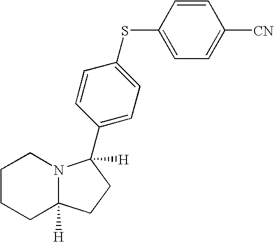 | 558 |

### Example 34

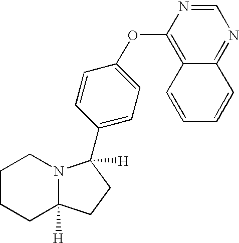

### 4-[4-(Octahydro-indolizin-3-yl)-phenoxy]-quinazoline Kᵢ=92 nM

¹H NMR (400 MHz, CDCl₃) δ 8.40 (m, 1H), 8.17 (s, 1H), 7.80 (m, 2H), 7.55 (m, 3H), 7.36 (d, J=8.3 Hz, 2H), 3.25 (t, J=8.3 Hz), 2.85 (bd, J=10.8 Hz, 1H), 2.12 (m, 2H), 1.75 (m, 4H), 1.60 (m, 4H), 1.30 (m, 2H).

### Example 39

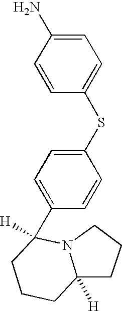

### 5-(4-(4-Aminophenylthio)phenyl)octahydroindolizine Kᵢ=75 nM

The solution of 5-(4-(4-Nitrophenylthio)phenyl)octahydroindolizidine (the product of Example 27, 160 mg), and palladium on carbon (wt 10%, 10 mg) in methanol (50 mL) was hydrogenated (40 psi) for overnight. The reaction mixture was filtered and the filtrate concentrated in vacuo to give the title compound (145 mg).

### Example 40

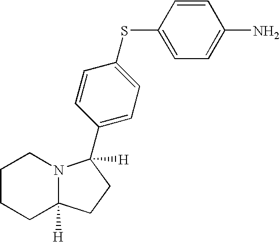

### tran-3-[4-(4-Aminophenylthio)phenyl]octahydroindolizine Kᵢ=62 nM

The title compound was prepared from Example 31 according to the procedure of Example 39.

¹H NMR (400 MHz, CDCl₃) δ 7.25 (m, 2H), 7.15-6.95 (m, 4H), 6.70 (m, 2H), 3.75 (bs, 2H), 2.95 (t, J=8.1 Hz, 1H), 2.65 (bd, J=10.7 Hz, 1H), 1.97 (m, 2H), 1.70 (m, 4H), 1.40 (m, 4H), 1.15 (m, 2H).

### Example 41

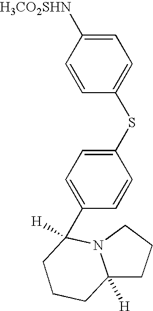

### 5-[4-(4-Methanesulfonaminophenylthio)phenyl]octahydroindolizine Kᵢ=75 nM

To the solution of 5-(4-(4-aminophenylthio)phenyl)octahydroindolizine (the product of Example 39, 33 mg, 0.102 mmol) in dichloromethane (10 mL) was added methanesulfonyl chloride (8.6 μL, 0.112 mmol), pyridine (9 μL, 0.112 mmol) and N,N-dimethylaminopyridine (1.2 mg, 0.0102 mmol). The mixture was stirred at room temperature for 2 days. The solvent was evaporated Preparative thin layer chromatography of the residue afforded the title compound (31 mg, 78%)

### Example 42

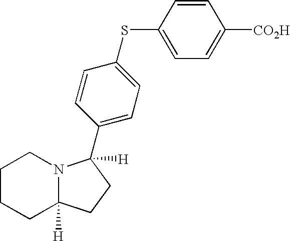

### tran-3-[4-(4-Carboxylicphenylthio)phenyl]octahydroindolizine Kᵢ=21 nM

The mixture of Example 38 (26 mg) in methanol (1 mL) and sodium hydroxide (2N, 10 mL) was heated at 80° C. for 16 h. The solvent was evaporated. The residue was dried by azeotroped with toluene. Thionyl chloride (0.1 mL) was added to a rapidly stirring suspension of the residue in Methanol. The mixture was stirred for 2 h. After concentration, the residue was purified by preparative thin layer chromatography to afford the title compound (5 mg).

¹H NMR (400 MHz, CDCl₃) δ 7.60 (m, 2H), 7.40 (m, 4H), 7.15 (m, 2H), 3.10 (t, J=8.1 Hz, 1H), 3.05 (bd, J=10.7 Hz, 1H), 2.07-1.97 (m, 2H), 1.79-1.72 (m, 4H), 1.55-1.41 (m, 4H), 1.27-1.18 (m, 2H).

### Example 43

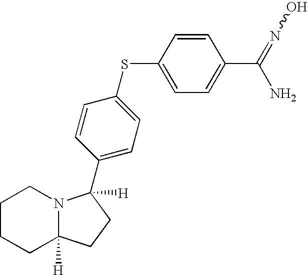

### tran-3-[4-((4-Amidoxime)phenylthio)phenyl]octahydroindolizine Kᵢ=40 nM

The mixture of Example 38 (56 mg), hydroxylamine hydrochloride (47 mg) and sodium carbonate (36 mg) in ethanol (10 mL) was heated at reflux temperature for 16 hours. The solvent was evaporated and the residue purified by preparative thin layer chromatography to give the title compound (15 mg).

¹H NMR (400 MHz, CDCl₃) δ 7.60-7.00 (m, 8H), 4.76 (bs, 1H), 3.05 (m, 1H), 2.70 (bd, J=10.7 Hz, 1H), 2.00 (m, 2H), 1.75 (m, 4H), 1.51 (m, 4H), 1.20 (m, 2H).

### Example 44

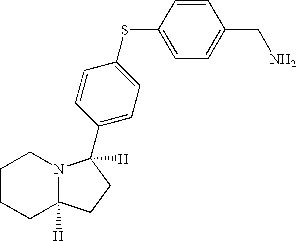

### tran-3-[4-(4-Methylaminophenylthio)phenyl]octahydroindolizine Kᵢ=9 nM

To the mixture of Example 38 (40 mg) in tetrahydrofuran (10 mL) was added lithium aluminum hydride (50 mg). After 1 h, water was added to quench the reaction. Extraction with ethyl acetate, concentration and purification via preparative thin layer chromatography afforded the title compound (10 mg).

¹H NMR (400 MHz, CDCl₃) δ 7.20 (m, 8H), 3.05 (t,), 2.68 (bd, J=10.8 Hz, 1H), 2.00 (m, 2H,), 1.75 (m, 4H), 1.48 (m, 4H), 1.20 (m, 2H).

### Example 45

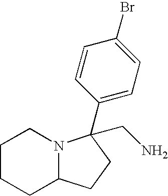

### 3-Methylamino-3-(4-bromophenyl)octahydroindolizine Kᵢ=5000 nM

To the solution of the product of Step C of Example 1 (6.3 g) in diethylether (200 mL) was added lithium aluminumhydride (3 g). The suspension was stirred at room temperature for 8 h. Water (10 mL) was added carefully. After filtration, drying and concentration, the crude product was obtained. Recrystallization from diethylether/ethylacetate afforded the desired compound (5 g).

### Example 46

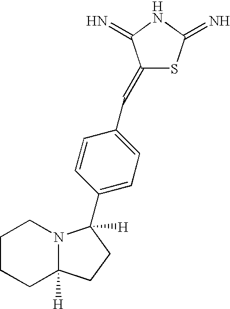

### trans-3-[4-(4-Methylene-1,3-thiazolidine-2,4-diimine)phenyl]octahydroindolizine Kᵢ=330 nM

A mixture of the trans-3-(4-formylphenyl)octahydroindolizine (the product of Step A of Example 23, 86 mg, 0.3755 mmol), 2-imino-1,3-thiazolan-4-one (40 mg, 0.3477 mmol), and sodium acetate (71 mg, 0.8693 mmol) in acetic acid (2 mL) was heated at reflux for 16 hours. After being cooled, water (10 mL) was added and the crude product precipitated. Washing with diethylether afforded the title compound (40 mg).

### Example 47

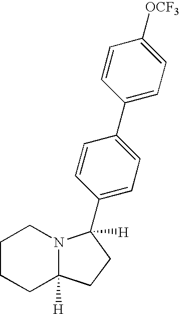

### trans-3-[4-(4-Trifluoromethoxyphenyl)phenyl]octahydroindolizine Kᵢ=1221 nM

A solution of the product of Example 1 (0.37 mmol) tri n-butylphosphine (0.013 mmol), 1,4-bis-(dibenzylideneacetone)palladium(0) (0.0055 mmol), 4-trifluoroxyphenyl boronic acid (0.39 mmol), cesium carbonate (0.74 mmol) in dioxane (0.4 mL) was heated to 80° C. for 16 hours. Purification of the mixture by preparative thin layer chromatography afforded the title compound (85 mg).

The following compounds were prepared from Example 1 according to the procedure of Example 47 (Scheme 8). The reacting boronic acids and the resulting products are shown in Table 3. The products are racemates unless noted otherwise.

TABLE 3

| Example | Boronic Acid | Product | Kᵢ (nM) |
|---|---|---|---|
| 48 | 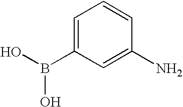 | 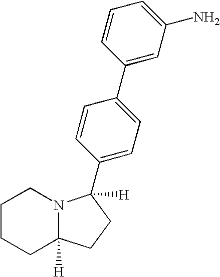 | 665 |
| 49 | 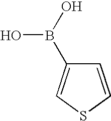 | 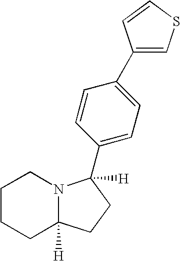 | 503 |
| 50 | 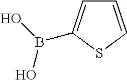 | 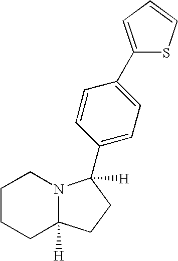 | 173 |
| 51 | 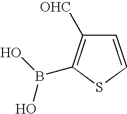 | 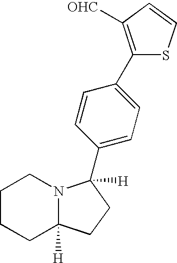 | 132 |
| 52 | 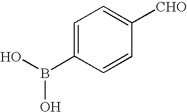 | 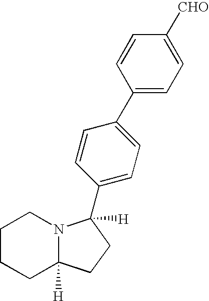 | 221 |
| 53 | 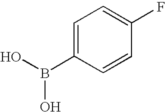 | 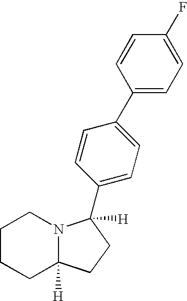 | 415 |

### Example 50

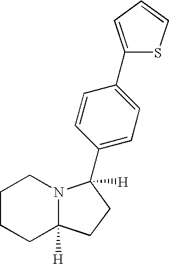

### 3-(4-Thiophen-2-yl-phenyl)-octahydro-indolizine Kᵢ=173 nM

¹H NMR (400 MHz, CDCl₃) δ 7.46 (m, 2H), 7.27 (d, J=8.1 Hz, 2H), 7.20 (dd, J=3.6, 0.87 Hz, 1H), 7.17 (dd, J=5.1, 1.2 Hz, 1H), 6.98 (dd, J=5.1, 3.6 Hz, 1H), 3.05 (td, J=8.2 Hz, 1H), 2.72 (bd, J=10.7 Hz, 1H), 2.00 (m, 2H), 1.60 (m, 4H), 1.51 (m, 4H), 1.20 (m, 2H).

### Example 54

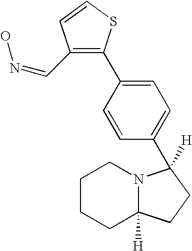

### tran-3-[4-(3-hydroxyiminomethylthienyl)phenyl]octahydroindolizine Kᵢ=1482

The mixture of the product of Example 51 (40 mg) and hydroxylamine hydrochloride (18 mg) in pyridine (1 mL) was stirred at room temperature for 16 hours. The pyridine was evaporated and preparative thin layer chromatography of the residue afforded the title compound (15 mg).

### Example 55

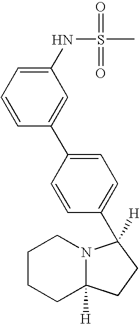

### tran-3-[4-(3-Methylsulfonylaminophenyl)phenyl]octahydroindolizine Kᵢ=320 nM

The title compound was prepared from Example 48 according to the procedure of Example 41.

### Example 56

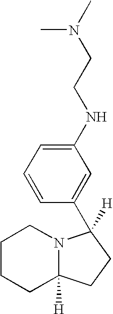

### tran-3-[4-(3-(2-Dimethylaminoethyl)amino)phenyl]octahydroindolizine Kᵢ=75 nM

A solution of trans-3-(3-bromophenyl)octahydroindolizine (the product of Example 21, 0.36 mmol), tri n-butylphosphine (0.0057 mmol), 1,4-(dibenzylideneacetone)palladium(0) (0.0071 mmol), 3-dimethylpropylamine (0.36 mmol), sodium t-butoxide (0.54 mmol) in dioxane (1 mL) was heated to 80° C. for 16 hours. Preparative thin layer chromatography of the mixture afforded the title compound (41 mg).

¹H NMR (400 MHz, CDCl₃) δ 7.10 (t, J=8.0 Hz, 1H), 6.60 (m, 2H), 6.40 (m, 1H), 3.10 (t, J=6.2 Hz, 2H), 3.00 (t, J=8.2 Hz, 1H), 2.78 (bd, J=10.9 Hz, 1H), 2.46 (t, J=6.2 Hz, 2H), 2.18 (s, 6H), 1.95 (m, 2H), 1.72 (m, 4H), 1.48 (m, 4H), 1.20 (m, 2H).

The following compounds were prepared according to the procedure of Example 56 (Scheme 8). The reacting amines and the resulting products are shown in Table 4. The products are racemic unless noted otherwise.

TABLE 4

| Example | Amine | Product | Kᵢ (nM) |
|---|---|---|---|
| 57 | 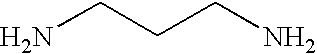 | 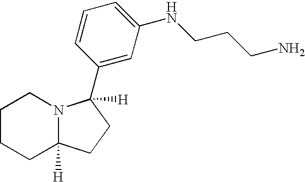 | N/A |
| 59 | 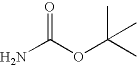 | 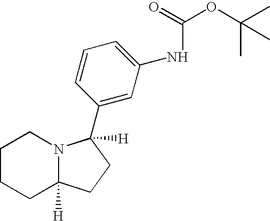 | N/A |

The following compounds were prepared according to Scheme 3 and 10. All the examples are isolated as racemates unless noted otherwise.

### Example 60

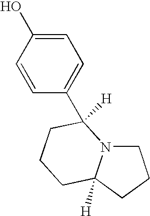

### 5-(4-Hydroxyphenyl)octahydroindolizine Kᵢ=1396 nM

A solution of 5-(4-bromophenyl)octahydroindolizine (the product of Step B of Example 26) (3.9 mmol, 1.1 g) in tetrahydrofuran (50 ml) was treated at −78° C. with n-butyllithium (2.5 M in hexanes, 4.7 mmol, 1.88 ml). The mixture was stirred at −78° C. for 1 hour, treated with trimethylborate (11.7 mmol, 1.2 ml) and allowed to warm to room temperature. An excess of N-methylmorpholine-N-oxide (11.7 mmol, 1.4 g) was then added to the solution under a positive pressure of nitrogen and the resulting suspension was heated at reflux temperature for 4 hours. After dilution with diethylether, the reaction mixture was treated with water and the organic phase washed with water to reach pH=7. The aqueous phases were extracted with diethylether and the combined organic phases were dried over magnesium sulfate, filtered, and concentrated. The residue was chromatographed on silica gel using a gradient of 1 to 20% of methanol (0.25 N of ammonia) in dichloromethane to give the title compound (0.41 g).

### Example 61

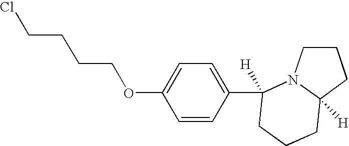

### 5-[4-(4-Chlorobutanoxy)phenyl]octahydroindolizine Kᵢ=278 nM

To a solution of 5-(4-hydroxyphenyl)octahydroindolidine (the product of Example 60, 0.23 mmol, 50 mg) in 8 ml of acetone was added 1-bromo-4-chlorobutane (0.23 mmol, 26%) and potassium carbonate (0.92 mmol, 127 mg). The mixture was stirred at 45° C. for 3 days. The title compound (52 mg) was obtained after purification via preparative thin layer chromatography eluting with 5% methanol (2N ammonia) in dichloromethane.

### Example 62

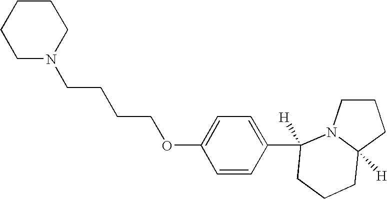

### 5-[4-(4-Piperidinylbutoxy)phenyl]indolizine Kᵢ=0.7 nM

To a solution of 5-[4-(4-chlorobutoxy)phenyl]indolizidine (the product of Example 61, 52 mg) in 8 ml of acetonitrile was added piperidine (0.23 mmol, 22.7 ml), and tetra-n-butylammonium iodide (0.014 mmol, 5 mg). The mixture was stirred at 60° C. for 2 days. The solvent was removed via vacuum, and the residue was purified by preparative thin layer chromatography eluting with 10% methanol (2N ammonia) in dichloromethane. The product was collected and washed with sodium bicarbonate, dried over sodium sulfate, filtered, and concentrated to give the title compound (7.2 mg).

¹H NMR (400 MHz, CDCl₃) δ 7.16 (d, J=8.6, 2H), 6.72 (d, J=8.6, 2H), 3.88 (t, J=6.4, 2H), 2.80 (dd, J=12.8, 2.7, 1H), 2.64 (m, 1H), 2.28 (m, 6H), 1.49-1.97 (m, 22H). ¹³C NMR (400 MHz, CDCl₃) δ 158.4, 137.1, 128.7, 114.5, 69.5, 68.1, 65.6, 59.5, 54.98, 53.1, 35.7, 31.3, 30.9, 27.9, 26.4, 25.6, 24.9, 23.9, 20.6.

### Example 63

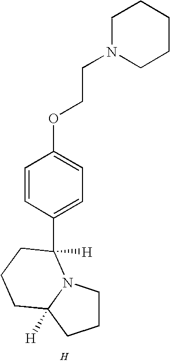

### 5-[4-(2-Piperidinylethanoxy)phenyl]octahydroindolizine Kᵢ=5 nM

To a solution of 5-(4-hydroxyphenyl)indolizidine (the product of Example 60, 0.12 mmol, 27 mg) in 8 ml of acetone was added 1-(2-chloroethyl)piperidine hydrochloride (0.37 mmol, 69 mg) and potassium carbonate (0.50 mmol, 69 mg). The mixture was stirred at 45° C. for 3 days, additional potassium carbonate (3.5 mmol, 484 mg) was added and the mixture heated at reflux temperature for one day. The title compound (17.1 mg) was isolated following preparative thin layer chromatography eluting with 5% methanol (2N ammonia) in dichloromethane.

¹H NMR (400 MHz, CDCl₃) δ 7.23 (d, J=8.6, 2H), 6.82 (d, J=8.6, 2H), 4.08 (t, J=6.1, 2H), 2.89-2.86 (dd, J=10.8, 2.8, 1H), 2.76 (t, J=6.1, 2H), 2.73-2.78 (m, 1H), 2.50 (br s, 1H), 1.98-1.22 (m, 18H).

### Example 64

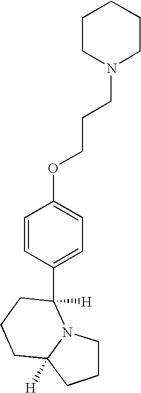

### 5-[4-(3-Piperidinylpropoxy)phenyl]octahydroindolizine Kᵢ=0.7 nM

### Step A 5-(4-Methoxyphenyl)-7(8H)-indolizinone

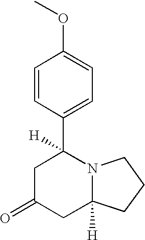

To a solution of 4-aminobutyraldehyde diethylacetal (50 mmol, 8.64 ml) in 50 ml of ethanol were added 25 ml of 3 N HCl, diethyl 1,3-acetonedicarboxylate (50 mmol, 9.1 ml), and p-anisaldehyde (50 mmol, 6.08 ml). The solution was allowed to stir at rt for 7 days. The mixture was neutralized with 10% aqueous potassium carbonate and extracted with diethylether. The diethylether was extracted with 125 ml of 6N HCl. The aqueous acid solution was heated at reflux temperature for 8 hours. The mixture was cooled, treated with sodium hydroxide to pH 7, and extracted with diethylether. The diethylether solution was dried over magnesium sulfate, filtered and concentrated. The residue was purified via silica gel chromatography eluting with 5% to 20% ethyl acetate in hexane. The title compound (425 mg) was collected after concentration.

### Step B 5-(4-Methoxyphenyl)octahydroindolizine

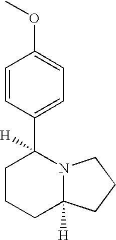

To a solution of the product of Step A (1.73 mmol, 425 mg) in 6 ml of diethylene glycol was added anhydrous hydrazine (3.46 mmol, 0.11 ml). The solution was heated at 95° C. for 1 h. A sample of potassium hydroxide (3.46 mmol, 194 mg) was added and the mixture heated until the temperature reached 230° C. whereupon distillation occurred. The distillate was collected, diluted with water and extracted with diethylether. The organic layer was dried over potassium carbonate, filtered and evaporated. The residue was purified via silica gel chromatography using 10% ethyl acetate in hexane. The title compound (60 mg), was collected after concentration.

### Step C 5-(4-Hydroxyphenyl)octahydroindolizine

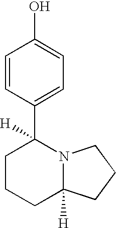

To a solution of 5-(4-methoxyphenyl)indolizidine (the product of Step B, 0.26 mmol, 60 mg) in acetic acid (0.26 ml) was added 48% HBr (0.91 ml) slowly. The mixture was stirred and heated at 100° C. for 8 hours. Evaporation of the solvent in vacuo afforded the title compound.

### Step D 5-(4-(3-Piperidinylpropoxy)phenyl)octahydroindolizine

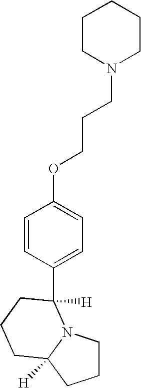

A mixture of 5-(4-hydroxyphenyl)octahydroindolizine (the product of Step C, 0.26 mmol, 56.5 mg), and sodium methoxide (1.56 mmol, 84 mg) in N,N-dimethylformamide (2 mL) was heated at 40° C. for 2 h. 1-Piperidinepropyl chloride (0.26 mmol, 52 mg) was added and the mixture heated at 80° C. for 8 hours. N,N-dimethylformamide was evaporated. Then water (25 mL) was added. The aqueous was extracted with dichloromethane (3×15 mL), and the organic portions dried over sodium sulfate, filtered and evaporated. The residue was purified via chromatography on alumina eluting with 10% ethyl acetate in hexane to give the title compound (25 mg).

¹H NMR (400 MHz, CDCl₃) δ 7.23 (d, J=8.6, 2H), 6.82 (d, J=8.7, 2H), 3.94 (t, J=6.4, 2H), 2.87 (dd, J=10.8, 2.8, 1H), 2.72-2.68 (m, 1H), 2.48-2.39 (m, 6H), 2.18-1.79 (m, 7H), 1.77-1.41 (m, 8H), 1.40-1.29 (m, 5H).

### Example 65

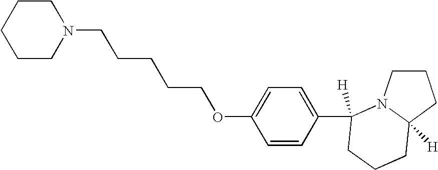

### 5-[4-(4-Piperidinylpentanoxy)phenyl]octahydroindolizine Kᵢ=1 nM

To a solution of 5-(4-hydroxyphenyl)octahydroindolizidine (0.103 mmol, 22.4 mg) in 5 ml of acetone was added 1-bromo-5-chlorobutane (0.103 mmol, 13.5 μl) and potassium carbonate (0.41 mmol, 57 mg). The mixture was stirred at 60° C. for 8 hours. Potassium carbonate (0.64 mmol, 89 mg) was added the next day, and the reaction mixture was heated at reflux temperature for an additional 8 hours. 1-Bromo-5-chlorobutane (0.103 mmol, 13.5 μl) was then added and the mixture heated at reflux temperature. Another portion 1-bromo-5-chlorobutane (0.06 mmol, 7 μl) was added after 8 hours and heating continued for 8 additional hours. Piperidine (0.309 mmol, 0.30 ml) and tetra-n-butylammonium iodine (0.007 mmol, 2 mg) were added and the mixture stirred at 60° C. for 8 hours. A second portion of piperidine (0.206 mmol, 0.2 ml) was added after 8 hours and stirring continued for 1 day. The reaction mixture was washed by sodium bicarbonate. The organic layer was collected, dried over sodium sulfate and concentrated. The title compound (6 mg) was obtained following purification via preparative thin layer chromatography on silica gel eluting with 10% methanol (2N ammonia) in dichloromethane.

¹H NMR (400 MHz, CDCl₃) δ 7.23 (d, J=8.5, 2H), 6.81 (d, J=8.7, 2H), 3.93 (t, J=6.5, 2H), 2.88 (m, 1H), 2.71 (m, 1H), 2.39-2.31 (m, 6H), 1.99-1.22 (m, 24H).

The following compounds were prepared according to Schemes 4 and 11. The products were isolated as racemates unless noted otherwise.

### Example 66

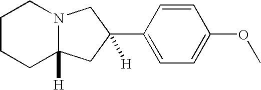

### anti-2-(4-Methoxyphenyl)octahydroindolizine

### See Example 67.

### Example 67

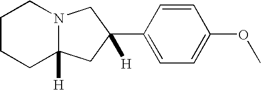

### syn-2-(4-Methoxyphenyl)octahydroindolizine

### Step A 2-(4-Methoxyphenyl)octahydroindolizine

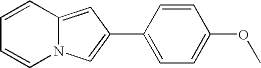

A solution of 2-bromo-4′-methoxyacetophenone (10 mmol, 2.29 g) and 2-picoline (10 mmol, 0.986 mL) in acetone (50 mL) was heated at reflux for 4 hours. The quaternary salt was precipitated salt was collected and redissolved in hot water (50 mL). Potassium carbonate (10 mmol, 1.38 g) was added. The mixture was heated at 80° C. for 8 hours. After filtration and drying in vacuo, the title compound (2.2 g) was collected.

### Step B anti- and syn-2-(4-Methoxyphenyl)octahydroindolizine

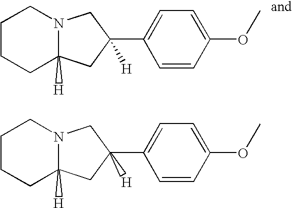

A suspension of the product of Step A (231 mg, 1 mmol) and platinum(IV) oxide (10 mg) in acetic acid (20 mL) was hydrogenated at 55 psi for 8 hours. The reaction mixture was filtered, and the solvent evaporated. The title compound was obtained in quantitative yield. (anti- and syn: 3.67:1). The two isomers were separated by preparative thin layer chromatography on silica (10% ethylacetate/dichloromethane) or preparative thin layer chromatography on alumina (5% ethylacetate/hexane).

### Example 68

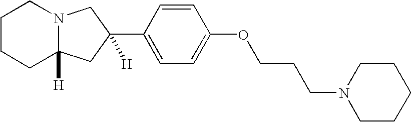

### anti-2-(4-(3-Piperidinylpropoxy)phenyl)octahydroindolizine Kᵢ=0.2 nM

The anti-2-(4-methoxyphenyl)octahydroindolizine (the product of Example 66, 46 mg, 0.199 mmol) was mixed with acetic acid (0.185 mL), and 48% HBr (0.74 mL). The mixture was heated at 100° C. for 2.5 hours. The solvent was evaporated and the residue dissovled in N,N-dimethylformamide (2 mL). 3-Piperidinylpropanyl chloride hydrochloride (40 mg, 0.199 mmol), and sodium methoxide (60 mg, 1.1 mmol) were added. The mixture was heated at 100° C. for 6 hours. The solvent was evaporated, and water (10 mL) was added. The water layer was extracted by dichloromethane (2×15 mL). After being dried, concentrated, preparative thin layer chromatography of the residue afforded the title compound (22 mg).

¹H NMR (400 MHz, CDCl₃) δ 7.05 (m, 2H), 6.72 (m, 2H), 3.92 (t, J=6.4 Hz, 2H), 3.32 (m, 2H), 3.05 (bd, J=10.9 Hz, 1H), 2.40 (m, 5H), 2.15-1.70 (m, 10H), 1.55 (m, 6H), 1.35 (m, 2H), 1.32 (m, 2H).

### Example 69

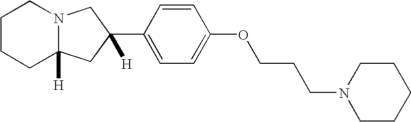

### syn-2-[4-(3-Piperidinylpropanoxy)phenyl]octahydroindolizine Kᵢ=0.2 nM

The syn-2-(4-methoxyphenyl)octahydroindolizine (the product of Example 67, 23 mg, 0.1 mmol) was mixed with acetic acid (0.09 mL), and 48% HBr (0.37 mL). The mixture was heated at 100° C. for 3 hour. The solvent was evaporated and the residue dissolved in N,N-dimethylformamide (2 mL). 3-Piperidinylpropanyl chloride hydrochloride (20 mg, 0.1 mmol), and sodium methoxide (60 mg, 1.1 mmol) were added. The mixture was heated at 100° C. for 6 hour. The solvent was evaporated, and water (10 mL) was added. The water layer was extracted with dichloromethane (2×15 mL). The organic extracts were dried and concentrated to give the title compound (32 mg).

¹H NMR (400 MHz, CDCl₃) δ 7.26 (m, 2H), 6.82 (m, 2H), 3.98 (t, J=6.4 Hz, 2H), 3.16 (m, 1H), 3.05 (m, 2H), 2.60 (t, J=9.4 Hz, 1H), 2.50-2.30 (m, 6H), 2.26 (m, 1H), 1.96 (m, 4H), 1.77 (m, 2H), 1.70-1.55 (m, 6H), 1.48 (m, 3H), 1.26 (m, 2H).

### Example 70

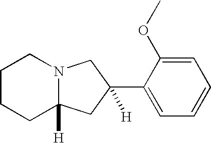

### anti-2-(2-Methoxyphenyl)octahydroindolizine Kᵢ=792 nM

### Step A 2-(2-Methoxyphenyl)indolizine

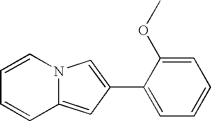

A solution of 2-bromo-2′-methoxyacetophenone (10 mmol, 2.29 g) and 2-picoline (10 mmol, 0.986 mL) in acetone (50 mL) was heated at reflux for 8 hours. The precipitate was collected and dissolved in hot water (50 mL). Potassium carbonate (10 mmol, 1.38 g) was added and the mixture heated at 80° C. for 3 days. Filtration and drying in vacuo gave the title compound (1.4 g).

### Step B anti-2-(2-Methoxyphenyl)octahydroindolizine

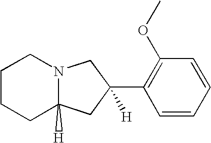

To a solution of the product of Step A (2.31 mmol, 517 mg) in 75 ml of acetic acid was added platinum(IV) oxide (26 mg, 5% by mass). The mixture hydrogenated at 50 psi for 8 hours whereupon the reaction mixture was filtered and the filtrate evaporated in vacuo The residue was dissolved in dichloromethane and washed with sodium bicarbonate solution. The organic layer was collected, dried over sodium sulfate and concentrated. The title compound (157 mg, 30%) was obtained following chromatography on silica gel eluting with 10% methanol (2N ammonia) in dichloromethane.

### Example 71

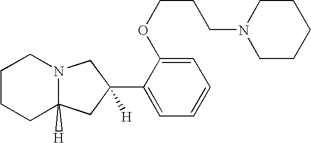

### anti-2-[2-(3-Piperidinylpropoxy)phenyl]octahydroindolizine Kᵢ=315 nM

### Step A anti-2-(2-Hydroxyphenyl)octahydroindolizine

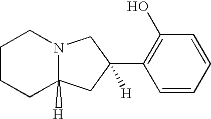

To a solution of anti-2-(2-methoxyphenyl)indolizine (the product of Example 70, 0.61 mmol, 140 mg) in acetic acid (0.6 ml) was added 48% HBr (2.1 ml) slowly. The mixture was stirred and heated at 100° C. for 8 hours. Solvent was removed in vacuo to give the title compound.

### Step B anti-2-[2-(3-Piperidinylpropanoxy)phenyl]octahydroindolizine

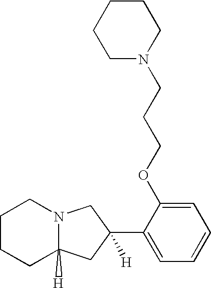

The mixture of the product of Step A (0.61 mmol, 140 mg), 1-piperidinepropanyl chloride (0.61 mmol, 121 mg), and sodium methoxide (1.8 mmol, 99 mg) in N,N-dimethylformamide (3 mL) was heated at 130° C. for 3 days. N,N-dimethylformamide was evaporated. Then water (30 mL) was added. After extraction with dichloromethane (3×20 mL), dried over sodium sulfate, evaporated. Purification via preparative thin layer chromatography on alumina using 5% ethyl acetate in hexane as eluant gave the title compound (6.2 mg).

### Example 72

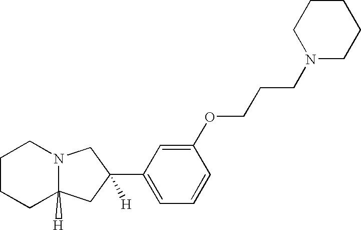

### anti-2-[3-(3-Piperidinylpropyloxy)phenyl]octahydroindolizine Kᵢ=0.3 nM

### Step A 2-(3-Methoxyphenyl)indolizine

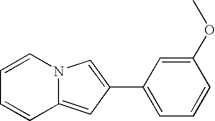

A solution of 2-bromo-3′-methoxyacetophenone (10 mmol, 2.29 g) and 2-picoline (10 mmol, 0.986 mL) in acetone (50 mL) was heated at reflux for 8 hours. The precipitated salt was collected and redissolved in hot water (50 mL). Potassium carbonate (10 mmol, 1.38 g) was added and the mixture heated at 80° C. for 3 days. Filtration and drying in vacuo gave the title compound (1.35 g).

### Step B 2-(3-Hydroxyphenyl)indolizine

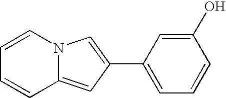

A mixture of the product from Step A (3.65 mmol, 0.815 g) and sodium ethanthiolate (7.3 mmol, 0.768 g) in N,N-dimethylformamide (22 mL) was heated at 80° C. for 8 hours. N,N-dimethylformamide was evaporated and the residue dried in vacuo. Water (200 mL) was added, and pale white solid was formed. After filtration and drying in vacuo, the title compound (0.44 g) was collected.

### Step C 2-(3-Piperidinopropoxyphenyl)indolizine

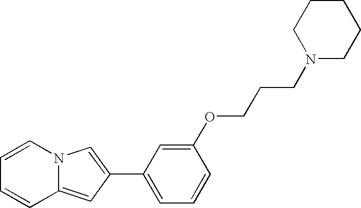

The mixture of the product of Step B (1.47 mmol, 308 mg), 1-piperidinepropanyl chloride (1.47, 292 mg), and sodium methoxide (4.41 mmol, 238 mg) in N,N-dimethylformamide (10 mL) was heated at 80° C. for 8 h. N,N-dimethylformamide was evaporated. Then water (25 mL) was added. After extraction with dichloromethane (3×15 mL), drying over sodium sulfate, evaporation, the title compound was collected. Silica gel chromatography eluting with 0% to 100% methanol (2.0 N ammonia) in dichloromethane gave the title compound.

### Step D anti-2-(3-(3-Piperidinylpropanoxy)phenyl)octahydroindolizine

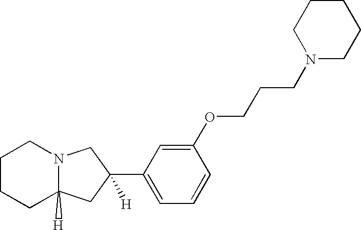

To a solution of the product of Step C (1.47 mmol, 492 mg) in 75 ml of acetic acid was added platinum(IV) oxide (25 mg, 5% by mass). The mixture was placed under 55 psi of hydrogen for 8 hours. The reaction mixture was filtered and the solvent evaporated. The residue was dissolved in dichloromethane and washed with sodium bicarbonate solution. The organic layer was dried over sodium sulfate and concentrated in vacuo. The title compound (10.1 mg) was obtained after purification via preparative thin layer chromatography on alumina using 15% ethyl acetate in hexane as eluent.

¹H NMR (400 MHz, CDCl₃) δ 7.16 (t, J=7.8, 1H), 6.90 (m, 2H), 6.71-6.68 (m, 1H), 3.99 (t, J=6.4, 2H), 3.17-3.10 (m, 1H), 3.07-3.04 (m, 2H), 2.55 (t, J=9.4, 1H), 2.49-2.40 (m, 6H), 2.30-2.24 (m, 1H), 2.00-1.82 (m, 4H), 1.80-1.76 (m, 2H), 1.64-1.38 (m, 9H), 1.32-1.18 (m, 2H).

The following compounds were prepared according to Scheme 12. The products were isolated as racemates unless noted otherwise.

### Example 73

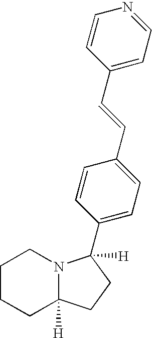

### trans-3-[4-(4-Pyridinylethenyl)phenyl]octahydroindolizine Kᵢ=65 nM

The mixture of Example 1 (0.28 g), 4-vinylpyridine (0.11 mL), palladium(II) acetate (0.022 g), tri-o-tolylphosphine (0.030 g) and triethylamine (0.14 mL) in acetonitrile (8 mL) was heated at reflux temperature for 12 hours. The reaction mixture was cooled to room temperature, filtered and the filtrate concentrated in vacuo. The residue was purified via silica chromatography (ethylacetate/hexanes) to give the title compound (0.035 g).

¹H NMR (400 MHz, CDCl₃) δ 8.52 (m, 3H), 7.45 (s, 1H), 7.32 (m, 2H), 7.25 (m, 2H), 6.91 (d, J=5.0 Hz, 2H), 3.19 (bs, 1H), 2.77 (bs, 1H), 2.08 (m, 2H), 1.79 (m, 4H), 1.55 (m, 4H), 1.26 (m, 2H).

### Example 74

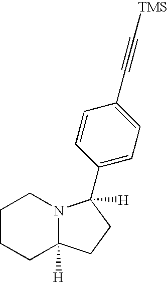

### trans-3-(4-trimethylsilylacetylenephenyl)octahydroindolizine Kᵢ=1500 nM

In a seal tube the mixture of Example 1 (0.28 g), trimethylsilylacetylene (0.21 mL), tetrakis(triphenylphosphine)palladium(0) (0.075 g), copper(I)iodide (0.007 g) and triethylamine (1.4 mL) in acetonitrile (5 mL) was heated at reflux temperature for 8 hours. The reaction mixture was cooled to room temperature, filtered and the filtrate concentrated in vacuo. The residue was purified via silica gel chromatography (ethylacetate/hexanes) to give the title compound (0.22 g).

### Example 75

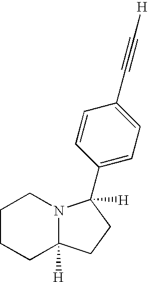

### trans-3-(4-acetylenephenyl)octahydroindolizine Kᵢ=1105 nM

The mixture of Example 74 (0.21 g) and potassium carbonate (0.095 g) in methanol (15 mL) was stirred at ambient temperature for 15 hours. The reaction mixture was concentrated in vacuo, the residue taken up in dichloromethane and washed with water (3×15 mL). The organic layer was dried over sodium sulfate, filtered and the filtrate concentrated in vacuo to give the title compound (0.15 g).

The following compounds were prepared according to Scheme 13. The products were isolated as racemates unless noted otherwise.

### Example 76

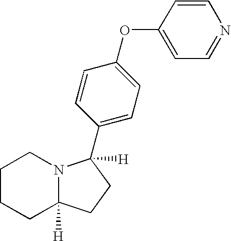

### trans-3-[4-(4-Pyridyloxy)phenyl]octahydroindolizine Kᵢ=77 nM

The mixture of Example 22 (0.14 g), 4-bromopyridine (0.10 mL) and potassium carbonate (0.10 g) in N,N-dimethylacetamide (8 mL) was heated at reflux temperature for 15 hours. The reaction mixture was cooled to room temperature and water (15 mL) was added. The resulting mixture was extracted with ethylacetate (15 mL) and the organic layer separated, washed with water (3×15 mL), dried over sodium sulfate, filtered and the filtrate concentrated in vacuo. The residue was purified via silica gel chromatography (ethylacetate/hexanes) to give the title compound (0.13 g).

¹H NMR (400 MHz, CDCl₃) δ 8.43 (dd, J=1.4 and 4.8 Hz, 2H), 7.36 (d, J=8.4 Hz, 2H), 7.01 (d, J=8.5 Hz, 2H), 6.80 (dd, J=1.6 and 4.8 Hz, 2H), 3.14 (t, J=8.1 Hz, 1H), 2.77 (d, J=10.7 Hz, 1H), 2.07 (m, 2H), 1.82 (m, 4H), 1.56 (m, 4H), 1.26 (m, 2H).

### Example 77

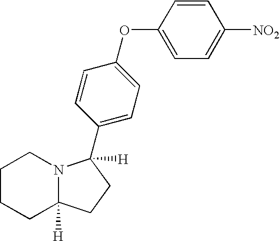

### trans-3-[4-(4-Nitrophenoxy)phenyl]octahydroindolizine Kᵢ=1564 nM

The mixture of Example 22 (0.2 g), 1-fluoro-4-nitrobenzene (0.097 mL) and potassium carbonate (0.2 g) in N,N-dimethylacetamide (8 mL) was heated at reflux temperature for 15 hours. The reaction mixture was cooled to room temperature and water (15 mL) was added. The resulting mixture was extracted with ethylacetate (15 mL) and the organic layer separated, washed with water (3×15 mL), dried over sodium sulfate, filtered and the filtrate concentrated in vacuo. The residue was purified via silica gel chromatography (ethylacetate/hexanes) to give the title compound (0.2 g).

### Example 78

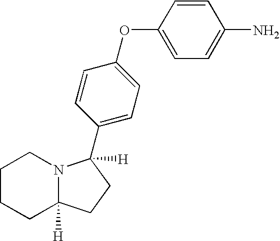

### trans-3-[4-(4-Aminophenoxy)phenyl]octahydroindolizine Kᵢ=296 nM

The mixture of Example 77 (0.1 g), palladium black (cat. 5% mol) and 1,4-cyclohexadiene (0.5 mL) in ethanol (5.0 mL) was heated at reflux temperature for 2 hours. The reaction mixture was cooled to room temperature and the precipitate filtered. The filtrate was concentrated in vacuo to give the title compound.

### Example 79

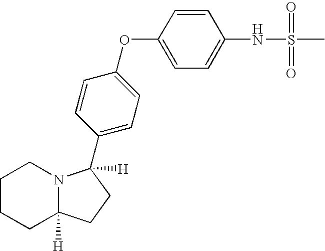

### trans-3-[4-(4-Methansulfonaminophenoxy)phenyl]octahydroindolizine Kᵢ=46 nM

To a solution of Example 78 (0.02 g) in pyridine (2.0 mL) at 0° C. was slowly added methane sulfonylchloride (0.02 mL) and the reaction mixture stirred at ambient temperature for 4 hours. The solvent was removed and the residue purified via silica gel chromatography (ethylacetate) to give the title compound (0.005 g).

¹H NMR (400 MHz, CDCl₃) δ 7.31 (d, J=8.8 Hz, 2H), 7.20 (d, J=6.7 Hz, 2H), 6.99 (d, J=6.7 Hz, 2H), 6.95 (d, J=8.6 Hz, 2H), 6.32 (bs, 1H), 3.11 (t, J=8.0 Hz, 1H), 2.98 (s, 3H), 2.77 (d, J=10.7 Hz, 1H), 2.04 (m, 2H), 1.82 (m, 4H), 1.56 (m, 4H), 1.26 (m, 2H).

### Example 80

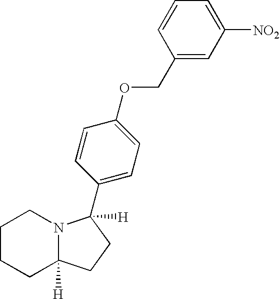

### trans-3-[4-(3-Nitrobenzyloxy)phenyl]octahydroindolizine MH⁺=353

A mixture of Example 22 (0.07 g), 3-nitrobenzylbromide (0.07 g) and potassium carbonate (0.06 g) in acetonitrile (8 mL) was heated at reflux temperature for 15 hours. The reaction mixture was cooled to room temperature, filtered, and the filtrate concentrated in vacuo. The residue was purified via silica gel chromatography (ethylacetate/hexanes) to give the title compound (0.1 g).

### Example 81

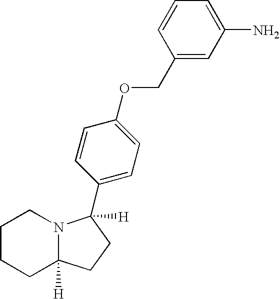

### trans-3-[4-(3-Aminobenzyloxy)phenyl]octahydroindolizine MH⁺=323

A mixture of Example 80 (0.09 g) and tin (II) chloride dihydrate (0.2 g) in ethanol was heated at reflux temperature for 2 hours. The reaction mixture was cooled to room temperature and concentrated in vacuo. The residue was taken up in dichloromethane and washed with saturated solution of sodium carbonate and water. The organic layer was dried over sodium sulfate, filtered and the filtrate concentrated in vacuo to give the title compound.

### Example 82

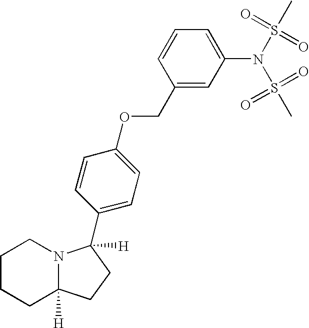

### trans-3-[4-(3-bis-Methansulfonaminobenzyloxy)phenyl]octahydroindolizine Kᵢ=128 nM

To a solution of Example 81 (0.08 g) in pyridine (3.0 mL) at 0° C. was slowly added methane sulfonylchloride (0.06 mL) and the reaction mixture stirred at ambient temperature for 15 hours. The solvent was removed to give the title compound.

¹H NMR (400 MHz, CDCl₃) δ 7.59 (d, J=7.7 Hz, 1H), 7.51 (t, J=7.8 Hz, 1H), 7.44 (s, 1H), 7.32 (d, J=7.8 Hz, 1H), 7.25 (d, J=8.6 Hz, 2H), 6.91 (d, J=8.6 Hz, 2H), 5.07 (s, 2H), 3.41 (s, 6H), 3.09 (t, J=8.0 Hz, 1H), 2.77 (d, J=10.6 Hz, 1H), 2.04 (m, 2H), 1.81 (m, 4H), 1.53 (m, 4H), 1.28 (m, 2H).

### Example 83

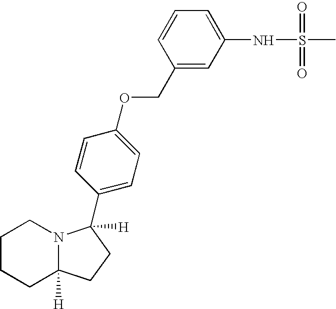

### trans-3-[4-(3-Methansulfonaminobenzyloxy)phenyl]octahydroindolizine Kᵢ=308 nM

A mixture of Example 82 (0.1 g) and 1N Sodium hydroxide (2.0 mL) in tetrahydrofuran (2.0 mL) was stirred at ambient temperature for 4 hours. Diethylether (10 mL) was added and the organic layer was separated, washed with water (3×15 mL), dried over sodium sulfate, filtered and the filtrate concentrated to give the title compound.

¹H NMR (400 MHz, CDCl₃) δ 7.37 (t, J=7.9 Hz, 1H), 7.26 (m, 4H), 7.19, (d, J=7.6 Hz, 1H), 6.90 (d, J=8.7 Hz, 2H), 5.04 (s, 2H), 3.04 (t, J=8.3 Hz, 1H), 2.74 (d, J=10.7 Hz, 1H), 2.01 (m, 2H), 1.78 (m, 4H), 1.55 (m, 4H), 1.26 (m, 2H).

### Example 84

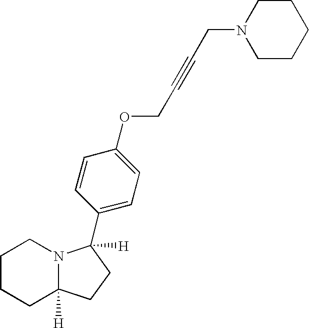

### trans-3-[4-(3-Piperidinylmethylpropargyloxy)phenyl]octahydroindolizine Kᵢ=1.8 nM

### Step A trans-3-(4-Propargyloxyphenyl)octahydroindolizine

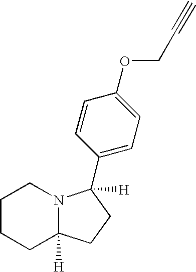

The mixture of Example 22 (0.606 g), propargyl bromide (0.331 mL) and potassium carbonate (0.771 g) in acetonitrile (10 mL) was heated at reflux temperature for 15 hours. The reaction mixture was cooled to room temperature, filtered, and the filtrate concentrated in vacuo. The residue was purified via silica gel chromatography (ethylacetate/hexanes) to give the title compound (0.558 g).

### Step B trans-3-[4-(3-Piperidinylmethyl propargyloxy)phenyl]octahydro-indolizine

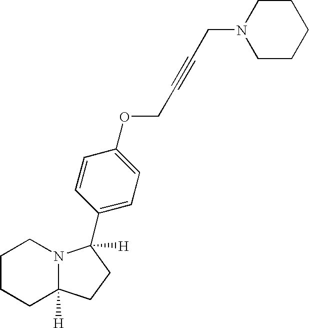

The mixture of the product of Step A, paraformaldehyde (0.656 g), copper(I) iodide (0.216 g) and piperidine (1.3 mL) in dioxane (20 mL) was stirred at 70° C. for 12 hours. The mixture was concentrated and then partitioned between dichloromethane and water. The organic layer was separated and washed with saturated ammonium chloride solution (4×25 mL). The organic layer was dried over sodium sulfate, filtered and concentrated. The residue was purified via silica gel chromatography (ethylacetate/hexanes) to yield the title compound (0.246 g).

¹H NMR (400 MHz, C₆D₆) δ 7.35-7.28 (m, 2H), 7.03-6.97 (m, 2H), 4.43 (t, J=2.0 Hz, 2H), 3.12-2.98 (m, 3H), 2.90-2.82 (m, 1H), 2.40-2.28 (m, 4H), 2.00-1.86 (m, 2H), 1.77-1.00 (m, 16H).

The following compounds were prepared according to Scheme 11. Unless noted otherwise the products are racemic.

### Example 85

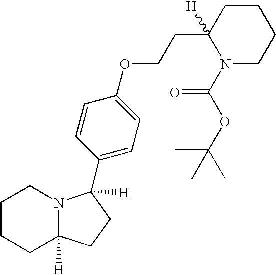

### trans-3-{4-[2,2′-(1-tert-Butylcarboxylatepiperidinyl)ethoxy]phenyl}-octahydroindolizine Kᵢ=51 nM

### Step A 2-(1-tert-Butylcarboxylatepiperidinyl)ethanol

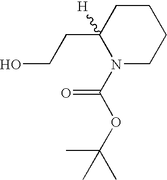

The mixture of 2-piperidine ethanol (2.0 g), di-tert-butyl-dicarboxylate (3.38 g) and triethylamine (2.4 mL) in methanol (77 mL) was stirred at 45° C. for 4 hours. The mixture was concentrated and partitioned between dichloromethane and saturated ammonium chloride solution. The organic layer was separated, dried over sodium sulfate, filtered and concentrated to give the title compound (3.42 g).

### Step B trans-3-{4-[2,2′-(1-tert-Butylcarboxylatepiperidinyl)ethoxy]phenyl}-octahydroindolizine

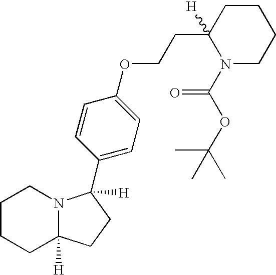

The mixture of Example 22 (1.0 g), the product of Step A (1.58 g), polymer-bound triphenylphosphine (3.07 g) and di-tert-butylazodicarboxylate (2.12 g) in dichloromethane (37 mL) was stirred at ambient temperature for 10 hours. The reaction mixture was filtered and the filtrate was concentrated in vacuo. The residue was purified via silica gel chromatography (ethylacetate/hexanes) to give the title compound (1.86 g).

1H NMR (400 MHz, C₆D₆) δ 7.41-7.31 (m, 2H), 7.00-6.89 (m, 2H), 4.71-4.51 (m, 1H), 3.92-3.66 (m, 2H), 3.11-3.01 (m, 1H), 2.95-2.86 (m, 1H), 2.64-2.45 (m, 1H), 2.09-1.84 (m, 3H), 1.81-1.05 (m, 27H).

### Example 86

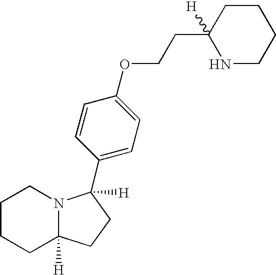

### trans-3-[4-(2,2′-piperidinylethoxy)phenyl]octahydroindolizine Kᵢ=90 nM

The solution of Example 85 (1.86 g) in dichloromethane (22 mL) was treated with trifluoroacetic acid (5 mL) and stirred at ambient temperature for 1 hour. The mixture was concentrated in vacuo and dissolved in water. The aqueous layer was neutralized with 1N sodium hydroxide solution and extracted with dichloromethane (6×10 mL). The combined organic extracts were dried over sodium sulfate, filtered and concentrated to afford the title compound (1.03 g).

¹H NMR (400 MHz, C₆D₆) δ 7.40-7.31 (m, 2H), 6.97-6.89 (m, 2H), 3.89-3.78 (m, 2H), 3.08 (t, J=8.1 Hz, 1H), 2.95-2.80 (m, 2H), 2.60-2.51 (m, 1H), 2.47-2.36 (m, 1H), 2.04-1.88 (m, 2H), 1.80-1.09 (m, 19H).

### Example 87

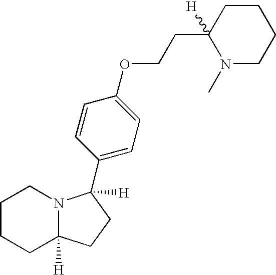

### trans-3-{4-[2,2′-(N-Methylpiperidinyl)ethoxy]phenyl}octahydroindolizine Kᵢ=7 nM

A solution of Example 86 (0.168 g) in formic acid (5 mL) was treated with p-formaldehyde (0.1 g) and stirred at 80° C. for 7 hours. The mixture was cooled to 0° C. and neutralized with 50% sodium hydroxide solution. The mixture was extracted with dichloromethane (6×10 mL) and the combined organic extracts dried over sodium sulfate, filtered and concentrated to afford the title compound (0.168 g).

¹H NMR (400 MHz, C₆D₆) δ 7.39-7.32 (m, 2H), 6.99-6.92 (m, 2H), 3.98-3.85 (m, 2H), 3.08 (t, J=8.3 Hz, 1H), 2.96-2.89 (m, 1H), 2.70-2.62 (m, 1H), 2.10 (s, 3H), 2.04-1.81 (m, 6H), 1.80-1.60 (m, 5H), 1.59-1.24 (m, 10H), 1.22-1.00 (m, 1H).

### Example 88

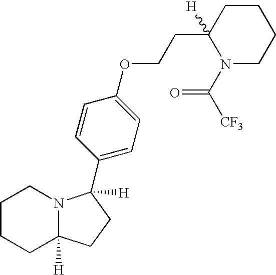

### trans-3-{4-[2,2′-(N-Trifluoroacetylpiperidinyl)ethoxy]phenyl}octahydroindolizine Kᵢ=71 nM

A solution of Example 86 (0.189 g) in dichloromethane (4 mL) was treated with trifluoroacetic anhydride (5 mL) and stirred at ambient temperature for 1 hour. The mixture was concentrated and then dissolved in dichloromethane. The organic layer was neutralized with Dowex® 550A basic resin, filtered and concentrated to yield the title compound (0.231 g).

¹H NMR (400 MHz, C₆D₆) δ 7.40-7.30 (m, 2H), 6.93-6.79 (m, 2H), 4.84-4.73 (m, 0.7H), 4.36-4.28 (m, 0.3H), 4.12-4.03 (m, 0.3H), 3.71-3.61 (m, 1.3H), 3.56-3.34 (m, 1.4H), 3.12-2.97 (m, 1H), 2.95-2.84 (m, 1H), 2.60-2.47 (m, 0.7H), 2.29-2.18 (m, 0.3H), 2.04-1.87 (m, 2H), 1.84-0.86 (m, 18H).

### Example 89

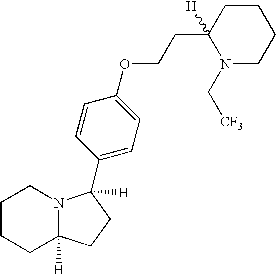

### trans-3-{4-[2,2′-(N-Trifluoroethylpiperidinyl)ethoxy]phenyl}octahydroindolizine Kᵢ=47 nM

A solution of Example 88 (0.139) in tetrahydrofuran (2 mL) was treated with borane (1.64 mL, 1.0M borane in tetrahydrofuran) and stirred at 67° C. for 12 hours. The mixture was concentrated and then dissolved in 4 mL of 1N HCl and stirred at 100° C. for 2 hours. The mixture was neutralized with 25% sodium hydroxide solution and extracted with dichloromethane (6×10 mL). The combined organic extracts were dried over sodium sulfate, filtered and concentrated to yield the title compound (0.128 g).

¹H NMR (400 MHz, C₆D₆) δ 7.42-7.34 (m, 2H), 6.98-6.91 (m, 2H), 3.79-3.65 (m, 2H), 3.09 (t, J=8.1 Hz, 1H), 2.95-2.88 (m 1H), 2.79-2.50 (m, 4H), 2.29-2.21 (m, 1H), 2.04-1.89 (m 2H), 1.84-0.98 (m, 18H).

### Example 90

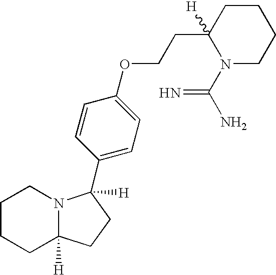

### trans-3-{4-[2,2′-(N-Amidinopiperidinyl)ethoxy]phenyl}octahydroindolizine Kᵢ=80 nM

### Step A

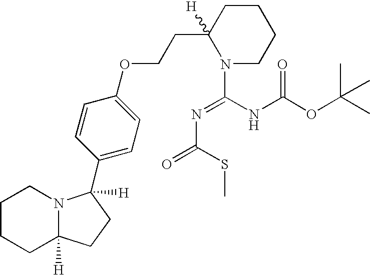

A mixture of Example 86 (0.115 g), 1,3-bis(tert-butoxycarbonyl)-2-methyl-2-thiopseudourea (0.1 g), copper(II) chloride (0.94 g) and Et₃N (0.291 g) in N,N-dimethylformamide (2 mL) was stirred at 60° C. for 48 hours. The mixture was diluted with ethylacetate and washed with water. The organic layer was separated, dried over sodium sulfate, filtered and concentrated. The residue was purified via silica gel chromatography (ethylacetate/hexanes) to yield the title compound (0.094 g).

### Step B trans-3-{4-[2,2′-(N-Amidinopiperidinyl)ethoxy]phenyl}-octahydroindolizine

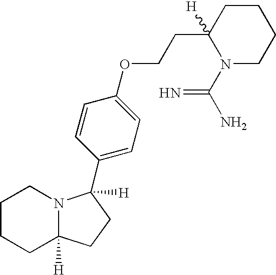

A solution of the product of Step A (0.094 g) in dichloromethane (4 mL) was treated with 1 N HCl (10 mL) and stirred at 110° C. for 12 hours. The mixture was cooled to ambient temperature and neutralized with 50% sodium hydroxide solution. The aqueous layer was extracted with dichloromethane (6×10 mL) and the combined organic extracts were dried over sodium sulfate, filtered and concentrated to yield the title compound without further purification (0.052 g).

¹H NMR (400 MHz, CDCl₃) δ 7.43-7.32 (m, 2H), 7.00-6.90 (m, 2H), 3.90-3.74 (m, 2H), 3.13-3.04 (m, 1H), 2.96-2.82 (m, 2H), 2.71-2.39 (m, 2H), 2.05-1.89 (m, 2H), 1.80-0.80 (m, 21H).

### Example 91

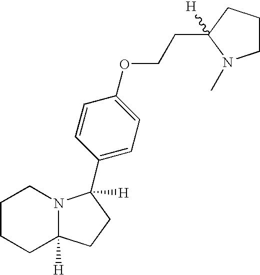

### trans-3-{4-[2,2′-(N-Methylpyrrolidinyl)ethoxy]phenyl}octahydroindolizine Kᵢ=2 nM

The mixture of Example 22 (0.5 g), N-methyl-2-pyrrolidine-2-ethanol (0.47 mL), polymer-bound triphenylphosphine (1.53 g) and di-tert-butylazodicarboxylate (1.06 g) in dichloromethane (18 mL) was stirred at ambient temperature for 10 hours. The reaction mixture was filtered and the filtrate was concentrated in vacuo. The residue was purified via silica gel chromatography (ethylacetate/methanol) to give the title compound (0.266 g).

¹H NMR (400 MHz, C₆D₆) δ 7.40-7.32 (m, 2H), 6.97-6.89 (m, 2H), 3.88-3.74 (m 2H), 3.08 (t, J=8.3 Hz, 1H), 3.00-2.88 (m, 2H), 2.19-1.88 (m, 8H), 1.80-1.30 (m, 14H), 1.23-1.09 (m, 1H).

### Example 92

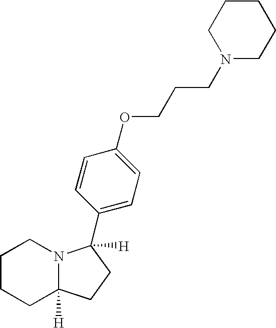

### trans-3-(4-(3-Piperidinylpropoxy)phenyl)octahydroindolizine Kᵢ=0.3 nM

### Step A 3-Piperidine propanol

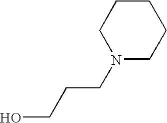

To piperidine (21 mL) was added slowly 1-bromopropanol (6.5 mL) at 0° C. and the reaction mixture was stirred at ambient temperature for 14 hours. Diethylether (20 mL) was added and the white precipitate was removed by filtration. The filtrate was washed with water (3×20 mL), dried over sodium sulfate, filtered and concentrated in vacuo to give the title compound (10 g).

### Step B trans-3-(4-(3-Piperidinylpropoxy)phenyl)octahydroindolizine

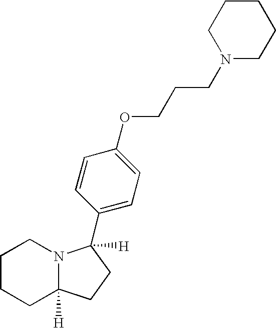

The mixture of Example 22 (0.8 g), the product of Step A (0.8 g), polymer-bound triphenylphosphine (2.4 g) and di-tert-butylazodicarboxylate (1.3 g) in dichloro-methane (10 mL) was stirred at ambient temperature for 10 hours. The reaction mixture was filtered and the filtrate was concentrated in vacuo. The residue was purified via silica gel chromatography (methanol/dichloromethane) to give the title compound (0.7 g).

Example 92 was also prepared by the following procedure according to Scheme 2.

### Step A 4′-(3-Chloropropoxy)acetophenone

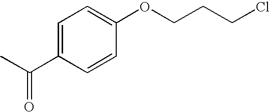

A mixture of p-hydroxyacetophenone (15 g) and 1-bromo-3-chloropropane (12 mL) in acetone (200 mL) was treated with potassium carbonate (17 g). The mixture was stirred at reflux temperature for 18 hours. The reaction was cooled to ambient temperature and filtered. The filtrate was concentrated in vacuo. The residue was dissolved in diethylether, washed with water, dried over sodium sulfate, filtered and concentrated in vacuo to yield the title compound (23 g).

### Step B trans-1-[4-(3-Chloropropoxy)phenyl]-3-(2-pyridyl)prop-2-en-1-one

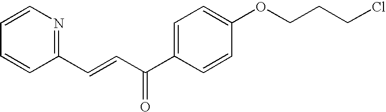

To a solution of 2-pyridine carboxaldehyde (19.5 mL) in 10% sodium hydroxide (25 mL) and methanol (35 mL) at 0° C. was added slowly the product of Step A (23 g) in methanol (15 mL). The reaction mixture was stirred at ambient temperature for 2 hours and the precipitate isolated by filtration and recrystallized from ethanol to give the title compound (35 g).

### Step C trans-3-(4-(3-chloropropoxy)phenyl)octahydroindolizine

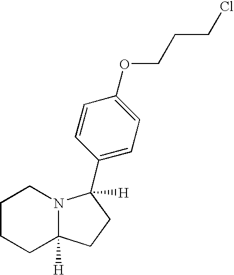

The product of Step B (14 g) and platinum(IV) oxide (0.5 g) in acetic acid (100 mL) and was hydrogenated at 55 psi and ambient temperature for 16 hours. The reaction mixture was filtered and the filtrate was concentrated in vacuo. The residue was dissolved in ethyl acetate (100 mL), washed with 1N sodium hydroxide (3×50 mL) and brine (50 mL), dried over sodium sulfate, filtered and concentrated in vacuo to yield the title compound (14 g).

### Step D trans-3-(4-(3-Piperidinylpropoxy)phenyl)octahydroindolizine

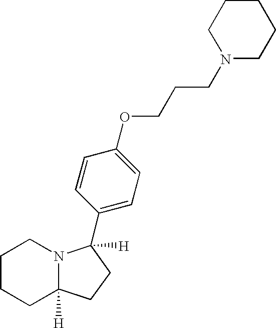

The product of Step C (13 g) and piperidine (50 mL) were heated at reflux temperature for 15 hours. The reaction was cooled to ambient temperature and the precipitate was removed by filtration. The filtrate was washed with water (3×50 mL) dried over sodium sulfate, filtered and concentrated in vacuo. The residue was purified via silica gel chromatography (methanol/dichloromethane) to give the title compound (7 g).

¹H NMR (400 MHz, CDCl₃) δ 7.23 (d, J=8.5 Hz, 2H), 6.84 (d, J=8.7 Hz, 2H), 3.98 (t, J=6.4 Hz, 2H), 3.04 (t, J=8.2 Hz, 1H), 2.74 (d, J=10.8 Hz, 1H), 2.46 (t, J=7.5 Hz, 2H), 2.39 (bs, 4H), 1.96 (m, 4H), 1.78 (m, 4H), 1.59 (m, 6H), 1.44 (m, 4H), 1.26 (m, 2H).

The product of Example 92 was resolved chromatographically using a Daicel AD column to afford the products of Examples 93 and 94.

### Example 93

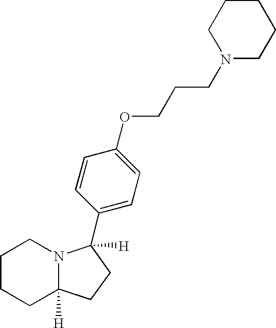

### (R, R)-3-(4-(3-Piperidinylpropoxy)phenyl)octahydroindolizine Kᵢ=0.06 nM

¹H NMR (400 MHz, CDCl₃) δ 7.23 (d, J=8.5 Hz, 2H), 6.84 (d, J=8.7 Hz, 2H), 3.98 (t, J=6.4 Hz, 2H), 3.04 (t, J=8.2 Hz, 1H), 2.74 (d, J=10.8 Hz, 1H), 2.46 (t, J=7.5 Hz, 2H), 2.39 (bs, 4H), 1.96 (m, 4H), 1.78 (m, 4H), 1.59 (m, 6H), 1.44 (m, 4H), 1.26 (m, 2H).

### Example 94

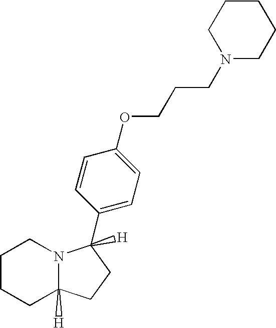

### (S, S)-3-(4-(3-Piperidinylpropoxy)phenyl)octahydroindolizine Kᵢ=0.06 nM

¹H NMR (400 MHz, C₆D₆) δ 7.38-7.30 (m, 2H), 6.99-6.90 (m, 2H), 3.84 (t, J=6.3 Hz, 2H), 3.07 (t, J=8.0 Hz, 1H), 2.95-2.86 (m, 1H), 2.34 (t, J=6.3 Hz, 2H), 2.24 (br s, 4H), 2.02-1.89 (m, 2H), 1.88-1.79 (m, 2H), 1.79-1.60 (m, 5H), 1.58-1.24 (m, 11H).

The following compounds were prepared according to Scheme 2.

### Example 95

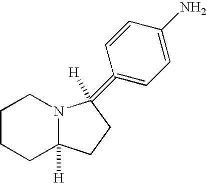

### trans-3-(4-Aminophenyl)octahydroindolizine Kᵢ=32 nM

### Step A: 1-(4-nitrophenyl)-3-(2-pyridinyl)-2-propen-1-one

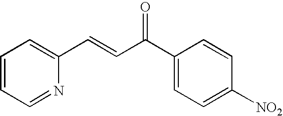

### 1-(4-nitrophenyl)-3-(2-pyridinyl)-2-propen-1-one

A mixture of 2-pyridinecarboxaldehyde (11.5 mL) and 4-nitroacetophenone (10 g) in ethylacetate (120 mL) was treated with a catalytic amount of sodium ethoxide (21% wt in ethanol). The mixture was stirred at ambient temperature for 2 hours. The mixture was concentrated and dissolved in dichloromethane, washed with water and then brine. The organic layer was dried over sodium sulfate, filtered and evaporated. The residue was recrystallized from ethylacetate to give the title compound (7.23 g).

### Step B trans-3-(4-Aminophenyl)octahydro indolizine

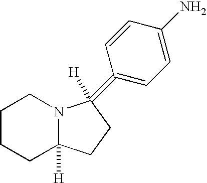

The product of Step A was dissolved in acetic acid (100 mL) and treated with platinum(IV) oxide (250 mg). The mixture was hydrogenated at 60 psi for 24 hours, filtered and concentrated. The residue was dissolved in dichloromethane and treated with Dowex® 550A basic resin. The reaction mixture was filtered and the filtrate evaporated The residue was purified via silica gel chromatography (ethylacetate/triethylamine) to give the title compound (2.97 g).

### Example 96

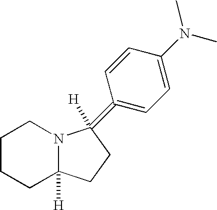

### trans-3-(4-(N,N-Dimethylamino)phenyl)octahydroindolizine Kᵢ=380 nM

A mixture of Example 95 (0.086 g) and p-formaldehyde (0.2 g) in formic acid (3 mL) was heated at 80° C. for 1 h. The mixture was cooled in an ice bath and neutralized with 50% sodium hydroxide solution. The mixture was extracted with dichloromethane (6×20 mL). The combined organic extracts were dried-over sodium sulfate, filtered and concentrated. The residue was purified via silica gel chromatography (ethylacetate) to give the title compound (0.018 g).

### Example 97

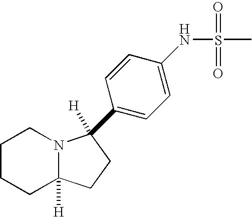

### trans-3-(4-(Methylsulfonylamino)phenyl)octahydroindolizine Kᵢ=481 nM

A mixture of Example 95 (0.106 g) and triethylamine (0.082 mL) in dichloromethane (3 mL) was treated with methanesulfonyl chloride (0.057 mL) and stirred at ambient temperature for 30 minutes. The mixture was treated with saturated sodium bicarbonate solution (5 mL). The organic layer was separated, dried over sodium sulfate, filtered and concentrated. The residue was purified via silica gel chromatography (ethylacetate/hexanes) to give the title compound (0.07 g) together with the product of Example 98 (0.024 g).

### Example 98

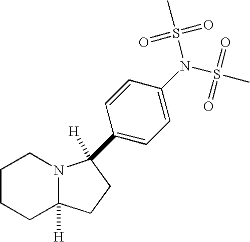

### trans-3-(4-(bis-Methylsulfonylamino)phenyl)octahydroindolizine Kᵢ=787 nM

### See Example 97.

### Example 99

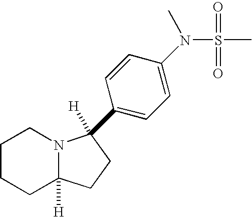

### trans-3-(4-(N-Methyl-N-methylsulfonylamido)phenyl)octahydroindolizine K_{I}=>5000 nM, MS (MH⁺ 309)

The product of Example 97 was dissolved in methanol (2 mL) and treated with trimethylsilyldiazomethane (0.05 mL, 2.0 M in hexanes). The mixture was stirred for 12 hours at ambient temperature then concentrated and purified via silica gel chromatography (ethylacetate/hexanes) to give the title compound (0.028 g).

The following compounds were prepared according to the procedures outlined in Scheme 14. The products were isolated as racemates unless noted otherwise.

### Example 100

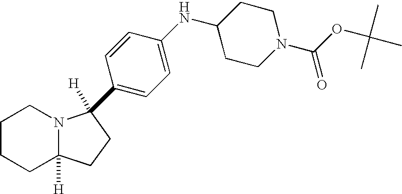

### trans-3-{4-[4-(N-(1,1-dimethylethoxycarbonyl)piperidinylamino]phenyl}octahydroindolizine Kᵢ=517 nM

A mixture of Example 95 (0.165 g), sodium triacetoxyborohydride (0.214 g), and tert-butyl-4-oxo-1-piperidinecarboxylate (0.144 g) in acetic acid 1,2-dichloroethane (0.04/4 mL) was stirred at ambient temperature for 12 hours. The mixture was treated with saturated sodium bicarbonate solution (8 mL), and the organic layer separated. The organic layer was dried over sodium sulfate, filtered and concentrated. The residue was purified via silica gel chromatography (ethylacetate/hexanes) to give the title compound (0.222 g)

### Example 101

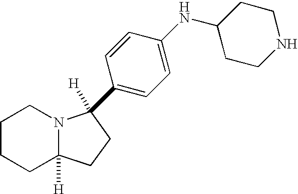

### trans-3-[4-(4-Piperidinylamino)phenyl]octahydroindolizine Kᵢ=314 nM

The product of Example 100 (0.178 g) was dissolved in methanol (2 mL) and treated with 4M HCl in dioxane (4.5 mL). The mixture was stirred for 30 minutes and concentrated. The residue was dissolved in methanol (10 mL) and treated with 1N sodium hydroxide solution. The mixture was extracted with dichloromethane (6×10 mL) and the combined organic extracts dried over sodium sulfate, filtered and concentrated to give the title compound (0.08 g).

### Example 102

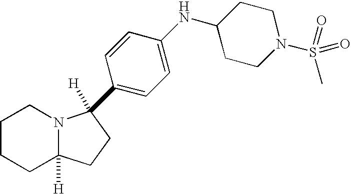

### trans-3-[4-(N-Methylsulfonyl-4-amino piperidine)phenyl]octahydro indolizine Kᵢ=243 nM

A mixture of the product of Example 101 (0.035 g) and triethylamine (0.025 mL) in dichloromethane (2 mL) was treated with methanesulfonyl chloride (0.009 mL) at 0° C. for 1 hour. The mixture was treated with saturated sodium bicarbonate solution (4 mL) and the organic layer separated. The organic layer was dried over sodium sulfate, filtered and concentrated to give the title compound (0.043 g).

¹H NMR (400 MHz, CDCl₃) δ 7.18-7.10 (m, 2H), 6.61-6.52 (m, 2H), 3.79-3.69 (m, 2H), 3.50-3.34 (m, 1H), 3.03-2.71 (m, 7H), 2.20-2.10 (m, 2H), 2.08-1.00 (m, 15H).

### Example 103

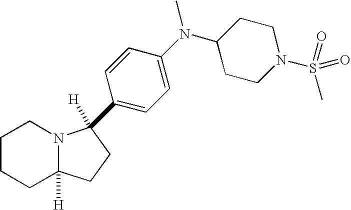

### trans-3-[4-(N-Methylsulfonyl)piperidinylamino)phenyl]octahydroindolizine Kᵢ=98 nM

A mixture of the product of Example 102 (0.014 g) and p-formaldehyde (0.1 g) in formic acid (2 mL) was heated at 80° C. for 3 hours. The mixture was cooled in an ice bath and neutralized with 50% sodium hydroxide solution. The mixture was extracted with dichloromethane (6×20 mL). The combined organic extracts were dried over sodium sulfate, filtered and concentrated to give the title compound (0.014 g).

¹H NMR (400 MHz, C₆D₆) δ 7.25-7.13 (m, 2H), 6.81-6.70 (m, 2H), 3.98-3.87 (m, 2H), 3.71-3.58 (m, 1H), 3.07-2.96 (m, 1H), 2.89-2.66 (m, 9H), 2.11-0.66 (m, 16H).

### Example 104

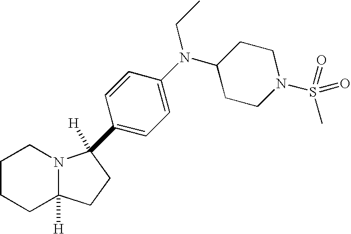

### trans-3-[4-(N-Ethyl-N-4-N-methylsulfonylpiperidinylamino)phenyl]octahydroindolizine Kᵢ=1776 nM

A mixture of the product of Example 102 (0.047 g) and acetaldehyde (0.007 mL) in acetic acid/1,2-dichloroethane (0.007/1 mL) was treated with sodium triacetoxyborohydride (0.037 g) and the mixture stirred for 30 minutes. The mixture was treated with saturated aqueous sodium bicarbonate solution (3 mL) and the organic layer separated. The organic layer was dried over sodium sulfate, filtered and concentrated. The residue was purified by silica gel chromatography (ethylacetate/hexanes) to give the title compound (0.036 g).

The following compounds were prepared according to the procedures outlined in Scheme 15. The products were isolated as racemates unless noted otherwise.

### Example 105

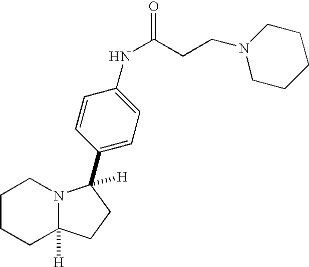

### trans-3-[4-(N-3-Piperidinylpropanamido)phenyl]octahydroindolizine Kᵢ=10 nM

A solution of Example 95 (0.130 g) in dichloromethane (3 mL) was treated with 1-(3-dimethylaminopropyl)-3-ethylcarbodiimide hydrochloride (0.127 g), 1-hydroxybenzotriazole (0.089 g), N,N-dimethylaminopyridine (0.081 g) and 1-piperidinepropionic acid (0.104 g). The mixture was stirred for 24 hours and then diluted with dichloromethane and washed with saturated ammonium chloride solution (2×10 mL) followed by saturated sodium bicarbonate solution (1×10 mL). The organic layer was dried over sodium sulfate, filtered and concentrated. The residue was purified via silica gel chromatography (ethylacetate) to afford the title compound (0.107 g).

¹H NMR (400 MHz, CDCl₃) δ 11.29 (br s, 1H), 7.50-7.44 (m, 2H), 7.31-7.24 (m, 2H), 3.08 (t, J=8.3 Hz, 1H), 2.79-2.71 (m, 1H), 2.68-2.40 (m, 7H), 2.10-1.95 (m, 2H), 1.87-1.13 (m, 17H).

### Example 106

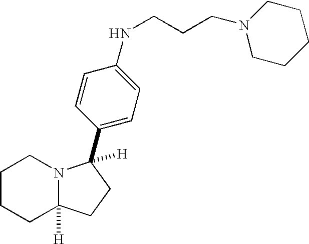

### trans-3-[4-(N-3-Piperidylpropylamino)phenyl]octahydroindolizine Kᵢ=1.5 nM

A solution of Example 105 in tetrahydrofuran (0.5 mL) was treated with borane (1 mL, 1M in tetrahydrofuran). The mixture was stirred at 68° C. for 24 hours and then concentrated. The residue was dissolved in 1N HCl and stirred at 100° C. for 12 hours. The mixture was cooled in an ice bath and neutralized with 25% sodium hydroxide solution. The aqueous layer was extracted with dichloromethane (6×5 mL) and the combined organic extracts dried over sodium sulfate, filtered and concentrated. The residue was purified via silica gel chromatography (ethylacetate/methanol) to afford the title compound (0.026 g).

¹H NMR (400 MHz, C₆D₆) δ 7.44-7.37 (m, 2H), 6.68-6.61 (m, 2H), 4.62 (br s, 1H), 3.11 (t, J=8.2 Hz, 1H), 3.06-2.98 (m, 3H), 2.30-2.10 (m, 5H), 2.09-1.91 (m, 2H), 1.83-1.64 (m, 5H), 1.62-1.10 (m, 14H).

### Example 107

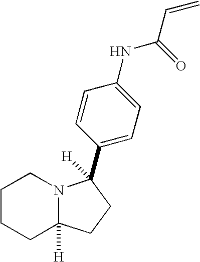

### N-[4-(trans-Octahydro-3-indolizinyl)phenyl]propenamide Kᵢ=327 nM

A mixture of Example 95 (0.161 g) and triethylamine (0.114 mL) in dichloromethane (4 mL) was treated with 3-chloropropionyl chloride (0.078 mL) at ambient temperature. The mixture was treated with saturated ammonium chloride solution (5 mL) and the organic layer separated. The organic layer was dried over sodium sulfate, filtered and concentrated. The residue was purified via silica gel chromatography (ethylacetate/hexanes) to afford the title compound (0.1 g).

### Example 108

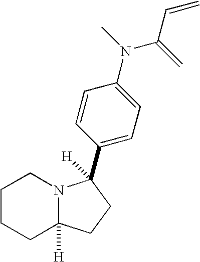

### N-Methyl-N-[4-(trans-Octahydro-3-indolizinyl)phenyl]propenamide Kᵢ=2770 nM

A mixture of Example 107 (0.95 g) and sodium hydride (0.022 g, 60% dispersion in mineral oil) was treated with iodomethane (0.024 mL). The mixture was stirred at ambient temperature for 1 hour and then treated with saturated ammonium chloride solution. The organic layer was separated, dried over sodium sulfate, filtered and concentrated. The residue was purified via silica gel chromatography (ethylacetate/hexanes) to afford the title compound (0.06 g).

### Example 109

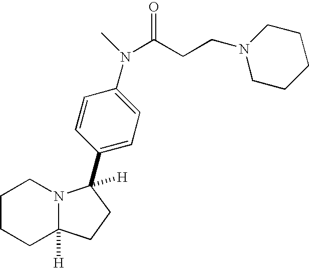

### N-Methyl-N-[4-(trans-Octahydro-3-indolizinyl)phenyl]-3-piperidinylpropenamide Kᵢ=1 nM

A solution of Example 108 (0.058 g) in toluene (1 mL) was treated with piperidine (0.03 mL). The mixture was stirred at 80° C. for 12 hours. The mixture was concentrated to afford the title compound (0.076 g).

¹H NMR (400 MHz, C₆D₆) δ 7.40-7.33 (m, 2H), 7.14-7.07 (m, 2H), 3.24 (s, 3H), 3.15 (t, J=8.0 Hz, 1H), 2.85-2.53 (m, 3H), 2.47-1.95 (m, 7H), 1.92-1.09 (m, 17H).

### Example 110

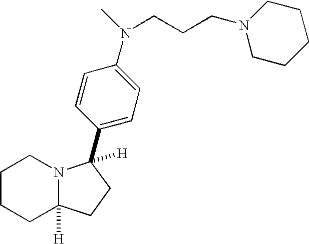

### trans-3-[4-(N-Methyl-N-3-piperidylpropylamino)phenyl]octahydroindolizine Kᵢ=2 nM

A solution of Example 109 in tetrahydrofuran (1 mL) was treated with borane (0.6 mL, 1M in tetrahydrofuran). The mixture was stirred at 68° C. for 24 hours and then concentrated. The residue was dissolved in 1N HCl and stirred at 100° C. for 12 hours. The mixture was cooled in an ice bath and neutralized with 25% sodium hydroxide solution. The aqueous layer was extracted with dichloromethane (6×5 mL) and the combined organic extracts dried over sodium sulfate, filtered and concentrated to yield the title compound (0.066 g).

¹H NMR (400 MHz, CDCl₃) δ 7.21-7.14 (m, 2H), 6.70-6.64 (m, 2H), 3.32 (t, J=7.3 Hz, 2H), 3.30-2.95 (m, 1H), 2.89 (s, 3H), 2.81-2.74 (m, 1H), 2.44-2.26 (m, 5H), 2.06-1.91 (m, 2H), 1.87-1.15 (m, 19H).

The following compounds were prepared according to the procedures outlined in Scheme 16.

### Example 111

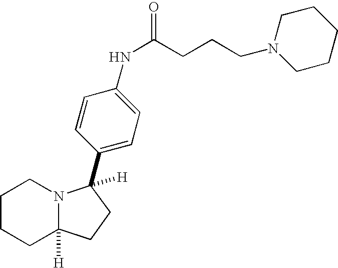

### trans-3-[4-(N-4-Piperidylbutanamido)phenyl]octahydroindolizine Kᵢ=3 nM

### Step A trans-3-[4-(N-4-Chlorobutanamido)phenyl]octahydroindolizine

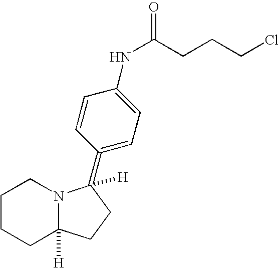

A solution of Example 95 (0.28 g) in dichloromethane (7 mL) was treated with 1-(3-dimethylaminopropyl)-3-ethylcarbodiimide hydrochloride (0.272 g), 1-hydroxybenzotriazole (0.293 g), N,N-dimethylaminopyridine, (0.173 g) and 4-chlorobutyric acid (0.140 mL). The mixture was stirred for 24 hours and then diluted with dichloromethane and washed with saturated ammonium chloride solution (2×10 mL) followed by saturated sodium bicarbonate solution (1×10 mL). The organic layer was dried over sodium sulfate, filtered and concentrated to give the title compound which was used without further purification.

### Step B trans-3-[4-(N-4-Piperidylbutanamido)phenyl]octahydroindolizine

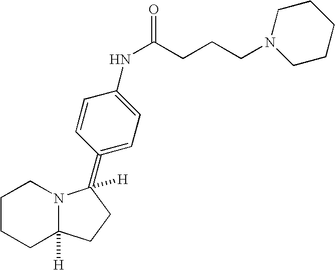

A solution of the product of Step A in toluene (6 mL) was treated with piperidine (0.2 mL). The mixture was stirred at 80° C. for 12 hours then concentrated and the residue dissolved in dichloromethane. The mixture was washed with saturated ammonium chloride solution (1×4 mL) and the organic layer separated, dried over sodium sulfate, filtered and concentrated. The residue was purified via silica gel chromatography (Ethylacetate/hexanes) to afford the title compound (0.033 g).

¹H NMR (400 MHz, C₆D₆) δ 8.27 (s, 1H), 7.88-7.77 (m, 2H), 7.47-7.34 (m, 2H), 3.05 (t, J=8.1 Hz, 1H), 2.94-2.84 (m, 1H), 2.20-1.86 (m, 12H), 1.80-1.06 (m, 26H).

### Example 112

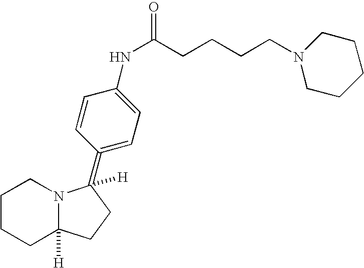

### trans-3-[4-(N-5-Piperidylpentanamido)phenyl]octahydro indolizine Kᵢ=2.1 nM

### Step A trans-3-[4-(N-5-Chloropentanamido)phenyl]octahydroindolizine

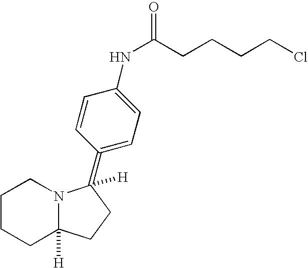

A solution of Example 95 (0.134 g) in dichloromethane (3 mL) was treated with 1-(3-dimethylaminopropyl)-3-ethylcarbodiimide hydrochloride (0.272 g), 1-hydroxybenzotriazole (0.092 g), N,N-dimethylaminopyridine, (0.083 g), and 5-chlorovaleric acid (0.069 mL). The mixture was stirred for 24 hours and then diluted with dichloromethane and washed with saturated ammonium chloride solution (2×10 mL) followed by saturated sodium bicarbonate solution (1×10 mL). The organic layer was dried over sodium sulfate, filtered and concentrated to give the crude product which was used without further purification.

### Step B trans-3-[4-(N-5-Piperidylpentanamido)phenyl]octahydroindolizine

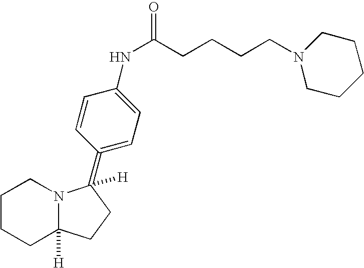

A solution of the product of Step A in toluene (6 mL) was treated with piperidine (0.42 mL). The mixture was stirred at 80° C. for 12 hours. The mixture was concentrated and the residue dissolved in dichloromethane, washed with saturated ammonium chloride solution (1×4 mL) and the organic layer separated. The organic layer was dried over sodium sulfate, filtered and concentrated. The residue was purified via silica gel chromatography (ethylacetate/methanol) to afford the title compound (0.181 g).

¹H NMR (400 MHz, C₆D₆) δ 7.84-7.77 (m, 2H), 7.61 (s, 1H), 7.42-7.35 (m, 2H), 3.04 (t, J=8.6 Hz, 1H), 2.91-2.84 (m, 1H), 2.28-2.13 (m, 6H), 2.09-2.03 (m, 3H), 1.98-1.86 (m, 2H), 1.78-1.08 (m, 19H).

### Example 113

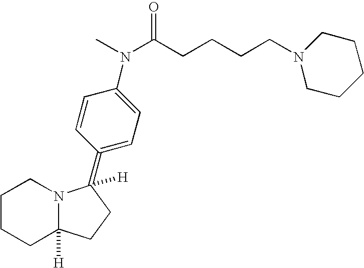

### trans-3-[4-(N-Methyl-N-5-piperidylpentanamido)phenyl]octahydroindolizine K_{I}=63 nM

A mixture of Example 113 (0.1 g) and sodium hydride (0.022 g, 60% dispersion in mineral oil) was treated with iodomethane (0.024 mL). The mixture was stirred at ambient temperature for 1 hour and then treated with saturated ammonium chloride solution. The organic layer was separated, dried over sodium sulfate, filtered and concentrated. The residue was purified via silica gel chromatography (ethylacetate/hexanes) to afford the title compound (0.05 g).

¹H NMR (400 MHz, C₆D₆) δ 7.32-7.23 (m, 2H), 6.89-6.74 (m, 2H), 3.17 (s, 3H), 3.06-2.94 (m, 1H), 2.85-2.67 (m 1H), 2.32-0.69 (m, 30H).

### Example 114

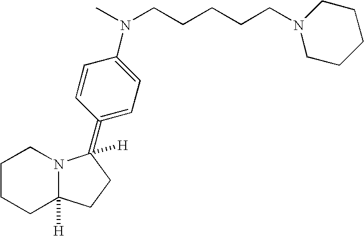

### trans-3-[4-(N-Methyl-N-5-piperidylpentylamino)phenyl]octahydroindolizine Kᵢ=9 nM

A solution of Example 114 (0.05 g) in tetrahydrofuran (1 mL) was treated with borane (0.6 mL, 1M in tetrahydrofuran). The mixture was stirred at 68° C. for 24 hours and then concentrated. The residue was dissolved in 1N HCl and stirred at 100° C. for 12 hours. The mixture was cooled in an ice bath and neutralized with 25% Sodium hydroxide solution. The aqueous layer was extracted with dichloromethane (6×5 mL) and the combined organic extracts dried over sodium sulfate, filtered and concentrated. The residue was purified via silcia gel chromatography (ethylacetate/methanol) to yield the title compound (0.004 g).

¹H NMR (400 MHz, C₆D₆) δ 7.46-7.41 (m, 2H), 6.76-6.71 (m, 2H), 3.16-2.99 (m, 4H), 2.63 (s, 3H), 2.33-2.20 (m, 3H), 2.17 (t, J=7.6 Hz, 2H), 2.10-1.91 (m, 2H), 1.84-1.63 (m, 6H), 1.62-1.09 (m, 17H).

### Example 115

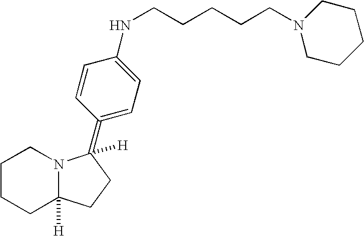

### trans-3-[4-(N-5-Piperidylpentylamino)phenyl]octahydroindolizine Kᵢ=0.7 nM

A solution of Example 113 (0.045 g) in tetrahydrofuran (1 mL) was treated with borane (3 mL, 1M in tetrahydrofuran). The mixture was stirred at 68° C. for 24 hours and then concentrated. The residue was dissolved in 1N HCl and stirred at 100° C. for 12 hours. The mixture was cooled in an ice bath and neutralized with 25% sodium hydroxide solution. The aqueous layer was extracted with dichloromethane (6×5 mL) and the combined organic extracts dried over sodium sulfate, filtered and concentrated. The residue was purified via silica gel chromatography (ethylacetate/methanol) to yield the title compound (0.015 g).

¹H NMR (400 MHz, C₆D₆) δ 7.40-7.32 (m, 2H), 6.56-6.48 (m, 2H), 3.10 (t, J=8.0 Hz, 1H), 3.06-2.97 (m, 1H), 2.86 (t, J=7.1 Hz, 2H), 2.29 (br s, 4H), 2.20 (t, J=7.3 Hz, 2H), 2.08-1.90 (m, 2H), 1.83-1.63 (m, 5H), 1.62-1.10 (m, 18H).

The following compounds were prepared according to the procedures outlined in Scheme 17. The products were isolated as racemates unless noted otherwise.

### Example 117

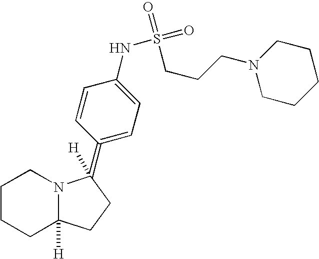

### trans-3-[4-(3-Piperidylsulfonylamino)phenyl]octahydroindolizine Kᵢ=4 nM

### Step A

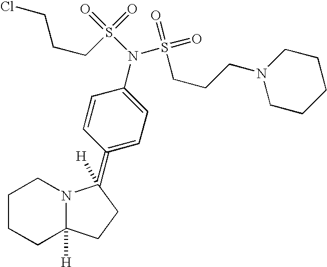

A mixture of Example 117 (0.143 g) and piperidine (2 mL) was stirred at 80° C. for 30 minutes. The mixture was concentrated and dissolved in dichloromethane (3 mL) and treated with triethylamine (0.044 mL) and 3-chloropropanesulfonyl chloride (0.035 mL). The mixture was stirred for 1 hour and then diluted with saturated sodium bicarbonate solution (6 mL) and the organic layer separated. The organic layer was dried over sodium sulfate, filtered and concentrated. The residue was purified via silica gel chromatography (Ethylacetate/hexanes) to afford the title compound (0.064 g).

### Step B trans-3-[4-(3-Piperidylsulfonylamino)phenyl]octahydroindolizine

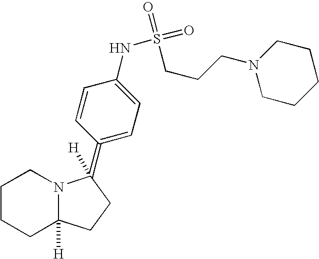

A solution of the product of Step A (0.064 g) in tetrahydrofuran (1 mL) was treated with 1N sodium hydroxide solution (1 mL). The mixture was stirred for 12 h at ambient temperature. The mixture was neutralized with 1N HCl solution and extracted with dichloromethane (6×5 mL). The combined organic extracts were dried over sodium sulfate, filtered and concentrated. The residue was purified via column chromatography (ethylacetate) to afford the title compound (0.017 g).

¹H NMR (400 MHz, CDCl₃) δ 7.39-7.11 (m, 4H), 3.20-3.05 (m, 3H), 2.82-2.38 (m, 6H), 2.19-1.08 (m, 22H).

### Example 118

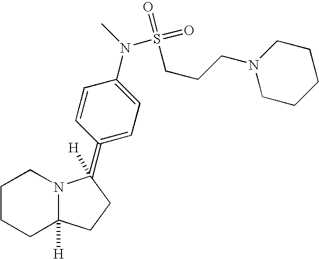

### trans-3-[4-(3-Piperidylsulfonyl-N-methylamino)phenyl]octahydroindolizine Kᵢ=61 nM

A solution of Example 117 (0.009 g) in methanol (1 mL) and N,N-diisopropylethylamine (0.008 mL) was treated with trimethylsilyidiazomethane (0.022 mL, 2.0 M in hexanes). The mixture was stirred for 12 hours at ambient temperature then concentrated and purified via silica gel chromatography (ethylacetate/hexanes) to give the title compound (0.007 g).

¹H NMR (400 MHz, CDCl₃) δ 7.41-7.28 (m, 4H), 3.33 (s, 3H), 3.20-3.07 (m, 3H), 2.83-2.43 (m, 6H), 2.24-2.00 (m, 4H), 1.89-1.40 (m, 17H).

### Example 119

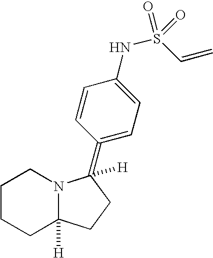

### trans-3-[4-(Vinylsulfonylamino)phenyl]octahydroindolizine Kᵢ=>500 nM, MS (MH⁺ 343)

A mixture of Example 95 (0.252 g) and triethylamine (0.18 mL) in dichloromethane (6 mL) was treated with 2-chloro-1-ethanesulfonyl chloride (0.122 mL). The mixture was stirred for 30 minutes and then diluted with saturated ammonium chloride solution (5 mL). The organic layer was separated, dried over sodium sulfate, filtered and concentrated. The residue was purified via silica gel chromatrography (ethylacetate/hexanes) to yield the title compound (0.206 g).

### Example 120

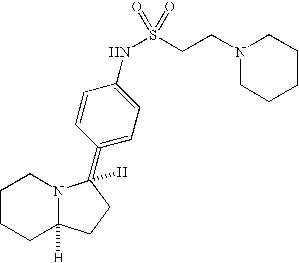

### trans-3-{4-[(2-Piperidylethyl)sulfonyl]amidophenyl}octahydroindolizine Kᵢ=11 nM

A solution of Example 119 (0.068 g) in toluene (1 mL) was treated with piperidine (0.04 mL). The mixture was stirred at 105° C. for 1 hour, cooled to ambient temperature, diluted with dichloromethane and saturated ammonium chloride solution (5 mL). The organic layer was separated, dried over sodium sulfate, filtered and concentrated. The residue was purified via silica gel chromatography (ethylacetate/hexanes) to yield the title compound (0.065 g).

¹H NMR (400 MHz, CDCl₃) δ 7.43-7.07 (m, 4H), 3.30-3.00 (m, 3H), 2.96-0.265 (m, 3H), 2.61-2.32 (m, 4H), 2.22-1.10 (m, 19H).

### Example 121

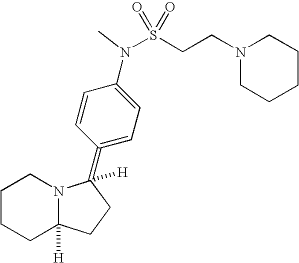

### trans-3-{4-[(2-Piperidylethyl)sulfonyl-N-methylamino]phenyl}octahydroindolizine Kᵢ=19 nM

A solution of Example 120 (0.036 g) in methanol (1 mL) and N,N-diisopropylethylamine (0.018 mL) was treated with trimethylsilyldiazomethane (0.05 mL, 2.0 M in hexanes). The mixture was stirred for 12 hours at ambient temperature then concentrated and purified via silica gel chromatography (ethylacetate/hexanes) to give the title compound (0.02 g).

¹H NMR (400 MHz, CDCl₃) δ 7.50-7.29 (m, 4H), 3.33 (s, 3H), 3.28-3.12 (m, 3H), 2.93-2.72 (m, 3H), 2.54-2.32 (m, 4H), 2.25-2.02 (m 2H), 2.00-1.00 (m, 16H).

### Example 122

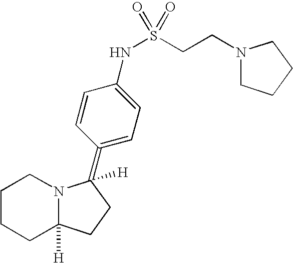

### trans-3-{4-[(2-Pyrrolidylethyl)sulfonylamino]phenyl}octahydroindolizine Kᵢ=534 nM

A solution of Example 120 (0.068 g) in toluene (1 mL) was treated with pyrrolidine (0.062 mL). The mixture was stirred at 105° C. for 1 hour. The mixture was cooled to ambient temperature, diluted with dichloromethane and saturated ammonium chloride solution (5 mL). The organic layer was separated, dried over sodium sulfate, filtered and concentrated. The residue was purified via silica gel chromatography (ethylacetate/methanol) to yield the title compound (0.046 g).

The following compounds were prepared according to the procedures outlined in Scheme 18. The products were isolated as racemates unless noted otherwise.

### Example 123

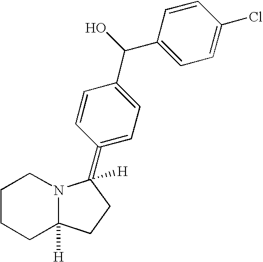

### trans-3-{4-[(4-Chlorophenyl)methan-1-ol]phenyl}octahydroindolizine Kᵢ=803 nM

A solution of trans-3-(4-bromophenyl)octahydroindolizine (Example 1, 0.42 g) in tetrahydrofuran (8 mL) was treated with n-butyllithium (1.12 mL, 2.0M in cyclohexane) at −78° C. and stirred for 45 minutes. The mixture was then treated with p-chlorobenzaldehyde (0.316 g) in tetrahydrofuran (8 mL) at −78° C. and stirred for 30 minutes. The mixture was allowed to warm to ambient temperature and treated with saturated ammonium chloride solution (8 mL). The organic layer was separated, dried over sodium sulfate, filtered and concentrated. The residue was purified via silica gel chromatography (ethylacetate/hexanes) to yield the title compound (0.415 g).

### Example 124

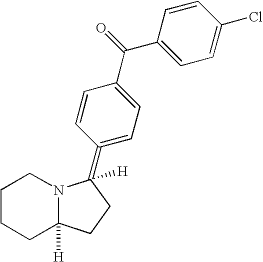

### trans-3-{4-[(4-Chlorophenyl)methan-1-oxo]phenyl}octahydroindolizine Kᵢ=368 nM

A solution of Example 123 (0.1 g) in dichloromethane (3 mL) was treated with manganese(II) oxide (0.250 g) and stirred at ambient temperature for 72 hours. The mixture was purified via silica gel chromatography (ethylacetate/hexanes) to afford the title compound (0.078 g).

### Example 125

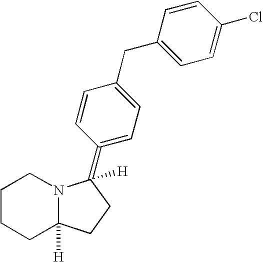

### trans-3-{4-[(4-Chlorobenzyl]phenyl}octahydroindolizine Kᵢ=655 nM

### Step A trans-3-{4-[(4-(1-Methylsulfonyloxy)-chlorobenzyl)]phenyl}-octahydroindolizine

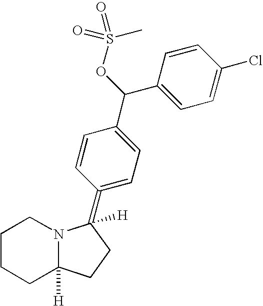

A mixture of Example 124 (0.13 g) and Et₃N (0.08 mL) in dichloromethane (4 mL) was treated with methanesulfonyl chloride (0.044 mL) at ambient temperature. The mixture was stirred for 10 minutes and then concentrated to afford the crude product which was used without further purification.

### Step B trans-3-{4-[(4-Chlorobenzyl]phenyl}octahydroindolizine

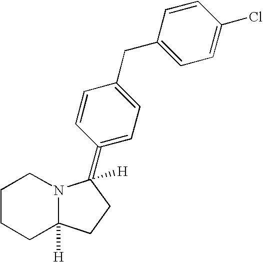

A solution of the product of Step A (0.16 g) in tetrahydrofuran (4 mL) was treated with lithium aluminum hydride (0.05 g) and the mixture stirred at 60° C. for 2 hours. The mixture was then treated with water (4 mL) and diluted with dichloromethane. The organic layer was separated, dried over sodium sulfate, filtered and concentrated. The residue was purified via silica gel chromatography (ethylacetate/hexanes) to afford the title compound (0.04 g).

### General Procedure for Examples 128-136

### Table 5 and Scheme 19

A mixture of Example 95 (1.0 equiv.) and sodium triacetoxyborohydride (1.4 equiv.) in acetic acid/1,2-dichloroethane (0.028/3 mL) was treated with the appropriate aromatic aldehyde (1.1 equiv.). The mixture was stirred at ambient temperature for 2 hours and then diluted with saturated sodium bicarbonate solution, and the organic layer separated. The organic layer was dried over sodium sulfate, filtered and concentrated. The residue was purified via silica gel chromatography (ethylacetate/hexanes) to yield the title compound.

TABLE 5

| Example | Starting Aldehyde | Product | Kᵢ (nM) |
|---|---|---|---|
| 127 | 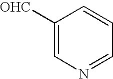 | 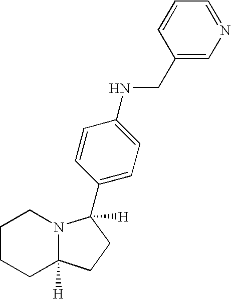 | 589 |
| 128 | 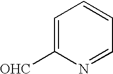 | 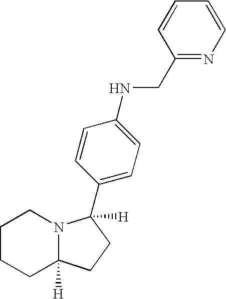 | 1338 |
| 129 | 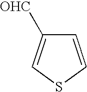 | 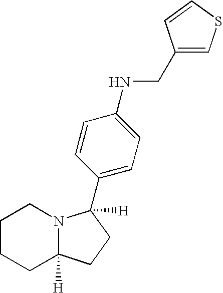 | 648 |
| 130 | 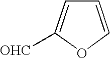 | 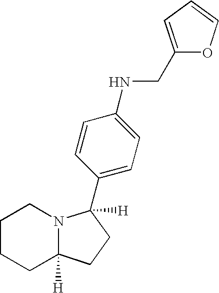 | 647 |
| 131 | 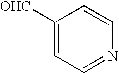 | 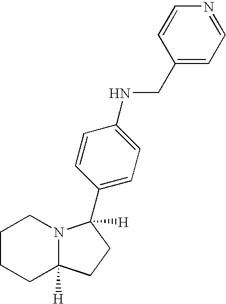 | 511 |
| 132 | 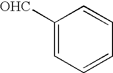 | 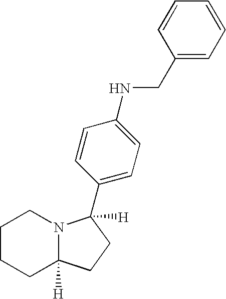 | 511 |
| 133 | 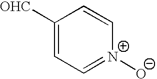 | 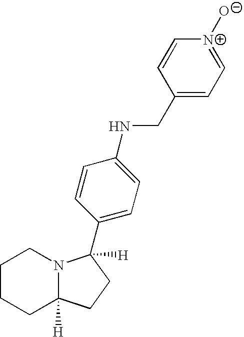 | 4384 |
| 134 | 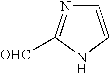 | 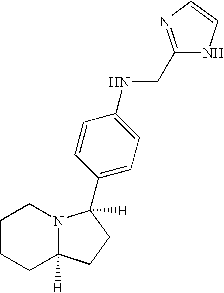 | 3644 |
| 135 | 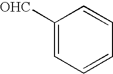 | 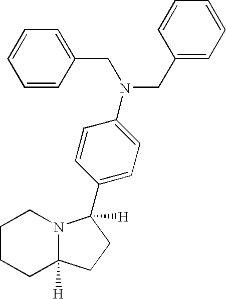 | 3947 |

### Example 136

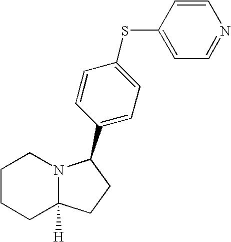

### Octahydro-3-[4-(4-pyridinylthio)phenyl]indolizine Kᵢ=7.5 nM

### Step A trans-3-(4-Bromophenyl)octahydroindolizine HCl

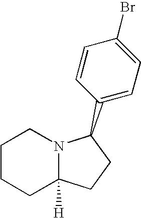

The title compound was prepared according to U.S. Pat. No. 4,683,239; Example 1. Enantiomerically pure compound may be prepared via resolution of the title compound using the appropriate di-para-toluoyl-tartaric acid as described in U.S. Pat. No. 4,683,239; Example 8, part b.

### Step B Octahydro-3-[4-(4-pyridinylthio)phenyl]indolizidine

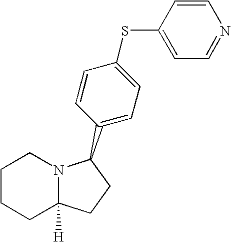

4-Mercaptopyridine (0.422 g) in n-butanol (20 mL) under dry nitrogen was treated with 60% sodium hydride (0.158 g) and stirred at ambient temperature for 1 hour. To this mixture was added a n-butanol solution (5 mL) of the product of Step A (1.06 g, free base) followed by tetrakis(triphenylphosphine)palladium(0) (0.437 g). The combined mixture was heated at reflux temperature for 20 hours, whereupon an additional portion of tetrakis(triphenylphosphine)palladium(0) (0.40 g) was added. After heating for a further 6 hours the reaction mixture was cooled to ambient temperature and the solvent evaporated to give an oil. The residue was partitioned between dichloromethane (100 mL) and water (100 mL). The organic portion was separated, washed with water (100 mL), dried over magnesium sulfate, filtered and evaporated. The crude product was purified by silica gel chromatography (dichloromethane/methanol/triethylamine) to give the title compound (0.71 g).

### Alternative Step B

50% Sodium hydride (1.01 g) was washed with hexane until free of oil, then suspended in N,N-dimethylformamide (20 mL). To this suspension was then added 4-mercaptopyridine (3.23 g) in N,N-dimethylformamide (35 mL). Once gas evolution had ceased a solution of the product of Step A (5.7 g, free base) in N,N-dimethylformamide (25 mL) was added followed by cuprous oxide (1.45 g). The combined mixture was heated at reflux for 18 hours and then treated with additional 4-mercaptopyridine (1.57 g) in N,N-dimethylformamide (12 mL), and 50% Sodium hydride (0.685 g), {hexane washed prior to addition}. Heating was continued for an additional 18 hours and then the solvent removed under reduced pressure. The residue was partioned between diethylether and water, and the organic layer separated, washed with water, dried over potassium carbonate, filtered and evaporated. The crude product was purified by silica gel chromatography (10% methylethylketone/hexane) to give the title compound (3.2 g). This material was treated with 70% perchloric acid (1.9 mL) to afford a hygroscopic solid which was dried in vacuo and recrystallized from Methanol to give the title compound (di-perchlorate), (3.89 g), m.p. 244.5-245.5°.

¹H NMR (400 MHz, CDCl₃) δ 8.30 (d, J=6.0 Hz, 2H), 7.46 (d, J=7.9 Hz, 2H), 7.42 (d, J=8.1 Hz, 2H), 6.91 (d, J=5.0 Hz, 2H), 3.19 (t, J=8.0 Hz, 1H), 2.77 (d, J=10.7 Hz, 1H), 2.09 (m, 2H), 1.80 (m, 4H), 1.53 (m, 4H), 1.27 (m, 2H).

The product of Example 136 was resolved chromatographically using a Daicel AD column eluting with hexane/1-propanol (95/5) containing diethylamine (0.1%) to afford the enantiomers.

### Example 137

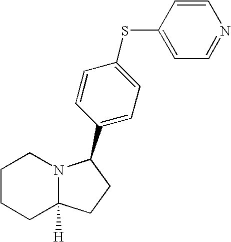

### (R, R)-Octahydro-3-[4-(4-pyridinylthio)phenyl]indolizine Kᵢ=3.8 nM

### Example 138

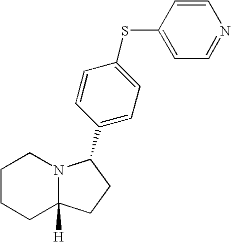

### (S, S)-Octa hydro-3-[4-(4-pyridinylthio)phenyl]indolizine Kᵢ=100 nM

### Example 140

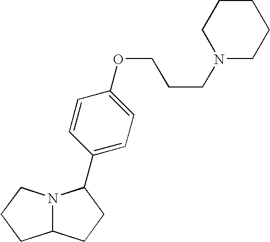

### 3-[4-(Piperidinylpropoxy)phenyl]hexahydro-1H-pyrrolizine Kᵢ=0.6 nM,

### Step A 4-(3-Chloropropoxy)iodobenzene

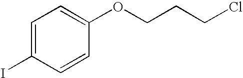

A suspension of 4-iodophenol (20 g), 3-chloro-1-bromopropane (18 mL), and potassium carbonate (38 g) in acetone (250 mL) was heated at reflux for 16 hours and allowed to cool to room temperature. The suspension was filtered, and the filtrate was evaporated in vacuo. Distillation of the residue (5-10 mm Hg, 2100) gave the title compound as a white crystalline solid (22 g).

### Step B 4-Piperidinylpropoxyiodobenzene

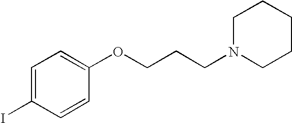

A suspension of the product of Step A (5 g), piperidine (2.2 mL), sodium carbonate (2.7 g), and potassium iodide (0.14 g) in n-butanol (30 mL) was heated in a 1050 bath for 18 hours. The resulting mixture was allowed to cool to room temperature, diluted with water (50 mL), and extracted with dichloromethane (2×20 mL). The combined organic phases were dried over magnesium sulfate and evaporated in vacuo. Distillation of the residue (5 mm Hg, 2600) gave the title compound as a white crystalline solid (4.8 g).

### Step C

The product of Step B (0.345 g) in diethylether (10 mL) was placed in a flame dried flask under nitrogen, cooled to −78° and treated with n-butyl lithium (1 mL, 2.5 M in hexane). The mixture was stirred at −78° for 45 minutes and at 0° for 5 minutes then cooled to −78°. To this solution was then added the product of Example 139 (0.125 g) in Et₂O (3 mL). The combined mixture was stirred at low temperature for 30 minutes, at −20° for 15 minutes, then warmed to ambient temperature.

In a second flask was placed aluminum trichloride (0.134 g) in diethylether (1 mL). This suspension was then added to a solution of lithium aluminum hydride (1 mL, 1M in tetrahydrofuran) in a third flask, which was then added to the reaction mixture, above. The combined mixture was stirred for 18 hours then treated with water (10 mL), stirred for 60 minutes and filtered. The residue was washed with ethylacetate (50 mL) and the filtrate and washings combined, washed with saturated sodium chloride solution, dried over magnesium sulfate, filtered and evaporated to give the title compound (0.24 g). MS (MH⁺ 329.2)

¹HNMR (400 MHz, CDCl₃), δ 7.27 (d, 2H, J=8.34 Hz), 6.84 (d, 2H, J=8.59 Hz), 3.98 (t, 2H, J=6.57 and 6.32 Hz), 3.71 (m, 1H), 3.57 (m, 1H), 2.85 (m, 1H), 2.61 (m, 1H), 2.42 (m, 6H), 2.13 (m, 2H), 1.39-2.00 (m, 14H).

### Biological Methods

### In Vitro

Transfection of Cells with Human Histamine Receptor

A 10 cm tissue culture dish with a confluent monolayer of SK-N-MC cells was split two days prior to transfection. Using sterile technique the media was removed and the cells were detached from the dish by the addition of trypsin. One fifth of the cells were then placed onto a new 10 cm dish. Cells were grown in a 37° C. incubator with 5% CO₂ in Minimal Essential Media Eagle with 10% Fetal Bovine Serum. After two days cells were approximately 80% confluent. These were removed from the dish with trypsin and pelleted in a clinical centrifuge. The pellet was then re-suspended in 400 μL complete media and transferred to an electroporation cuvette with a 0.4 cm gap between the electrodes (Bio-Rad #165-2088). One microgram of supercoiled H₃ receptor cDNA was added to the cells and mixed. The voltage for the electroporation was set at 0.25 kV, the capacitance was set at 960 μF. After electroporation the cells were diluted into 10 mL complete media and plated onto four 10 cm dishes. Because of the variability in the efficiency of electroporation, four different concentrations of cells were plated. The ratios used were; 1:20, 1:10, 1:5, with the remainder of the cells being added to the fourth dish. The cells were allowed to recover for 24 hours before adding the selection media (complete media with 600 μg/mL G418). After 10 days dishes were analyzed for surviving colonies of cells. Dishes with well isolated colonies were used. Cells from individual colonies were isolated and tested. SK-N-MC cells were used because they give efficient coupling for inhibition of adenylate cyclase. The clones that gave the most robust inhibition of adenylate cyclase in response to histamine were used for further study.

[³H]-N-methylhistamine binding

Cell pellets from histamine H₃ receptor-expressing SK-N-MC cells were homogenized in 20 mM TrisHCl/0.5 mM EDTA. Supernatants from a 800 g spin were collected, recentrifuged at 30,000 g for 30 minutes. Pellets were re-homogenized in 50 mM Tris/5 mM EDTA (pH 7.4). Membranes were incubated with 0.8 nM [³H]-N-methylhistamine plus/minus test compounds for 45 min at 25° C. and harvested by rapid filtration over GF/C glass fiber filters (pretreated with 0.3% polyethylenimine) followed by four washes with ice cold buffer. Filters were dried, added to 4 mL scintillation cocktail and then counted on a liquid scintillation counter. Non-specific binding was defined with 10 μM histamine. The pKᵢ values were calculated based on a K_{d} of 800 pM and a ligand concentration ([L]) of 800 pM according to the formula:

Kᵢ=(IC₅₀)/(1+([L]/(K_{d}))

### In Vivo

Elucidation of oral absorption and blood-brain barrier penetration profiles of H₃ receptor antagonists in the rat

A rat in vivo system is used to determine the blood-brain barrier penetration profiles and kinetics of various H₃ receptor antagonists after single bolus oral administration.

Female Sprague Dawley Rats (˜300 gram body weight) are housed in accordance with institutional standards and allowed to acclimate for at least 7 days prior to the study. Each H₃ antagonist is formulated in 0.5% hydroxypropylmethyl cellulose at a concentration of 1 mg/mL for oral dosing. The test compound is administered to each of eight animals as a single oral dose of 10 mL/kg (10 mg/kg). Remaining dosing solution is retained for analysis. Two animals from each original group of eight are euthanized via CO₂ asphyxiation at t=1, 6, 24, and 48 hours. After each animal is euthanized, 0.1 mL of its blood is sampled via cardiac puncture, and its brain is removed via dissection of the cranial bones and placed in a pre-weighed 50 mL conical tube on dry ice.

The blood is added to 0.3 mL of 6% trichloroacetic acid, and the acidified sample is vortexed and then centrifuged (5 minutes at 14,000 rpm in a microcentrifuge). The clear supernatant is retained for analysis. The frozen brain is weighed, homogenized in 6% trichloroacetic acid (3 mL/g wet weight of tissue), and then centrifuged. The clear supernatant is retained for analysis. The supernatants from the blood and brain samples are analyzed by liquid chromatography with mass spectral detection utilizing selective reaction monitoring (LC-MS/MS). The LC method uses a Phenomonex Polar RP column (2×50 mm) and a linear solvent gradient of water and acetonitrile (both 1% in acetic acid).

Graphs of H₃ receptor antagonist concentration versus time for blood and brain are generated from the LC-MS/MS results. The mean residency time (MRT) of the H₃ receptor antagonist, in blood or in the brain, is calculated from the ratio of the area under the first moment curve (AUMC) to the area under the concentration time curve (AUC): AUMC/AUC. The Blood Brain Barrier index is calculated from the log of AUC_{brain}/AUC_{blood}.

### Other Embodiments

The features and advantages of the invention will be apparent to one of ordinary skill in view of the discussion, examples, embodiments, and claims relating to the invention. The invention also contemplates variations and adaptations, based on the disclosure herein concerning the key features and advantages of the invention, and within the abilities of one of ordinary skill.

What is claimed is:

1. A compound of Formula (IA): 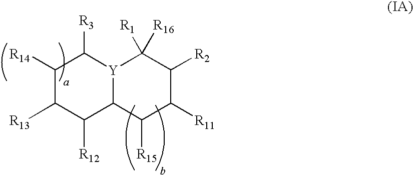
wherein:
a is 0 and b is 0;
or a is 1 and b is 0;
or a is 1 and b is 1;
Y is selected from N and N→O;
one of R₁, R₂ and R₃ is a ring moiety selected from C₄₋₆ cycloalkyl, phenyl, naphthyl, C₁₋₅ heterocyclyl, (C₄₋₆ cycloalkyl)C₁₋₃ alkylene, (phenyl)C₁₋₃ alkylene, (naphthyl) C₁₋₃ alkylene, and (C₁₋₅ heterocyclyl)C₁₋₃ alkylene;
and the remaining two of R₁, R₂ and R₃ are independently selected from hydrogen, halogen, and C₁₋₆ alkyl;
wherein said ring moiety is substituted with a moiety of formula:
—X—W-Z, X-Z, W-Z or Z;
wherein X is selected from the group consisting of O, S, SO₂, SO, NR₄, —CH═CH—, —C≡C—, —OCH₂—C≡C—, —C≡C—CH₂O—, —CH(R₅)—, CO, —O—CO—, —CO—O—, CHOH, —N—CO—, —CO—N—, —SO₂—NH—, —NR₄—SO₂—, and —SO₂—NR₄—; R₄ is H, or C₁₋₆ alkyl; R₅ is H, C₁₋₆ alkyl, or hydroxy;
W is C₁₋₆ alkylene, phenylene, (phenylene)(C₁₋₃ alkylene), or —CH₂—CHCH—CH₂—;
Z is selected from:
(i) NR₂₁R₂₂, NHCOR₂₃, or NHSO₂R₂₃,
(ii) C₃₋₆ heterocyclyl or C₇₋₁₂ fused bicyclyl, and
(iii) phenyl substituted with a C₃₋₆ heterocyclyl group, or with a (C₃₋₆ heterocyclyl)C₁₋₆ alkylene group,
wherein each phenyl or heterocyclyl group in (ii) or (iii) may be substituted with one to four substituents independently selected from the group consisting of halo, hydroxy, C₁₋₆ alkyl, C₁₋₆ alkoxy, cyclohexyl, cyclohexenyl, phenyl, (phenyl)C₁₋₆ alkylene, trihalo C₁₋₆ alkyl, nitro, SCH₃, NR₂₁R₂₂, amido, amidino, amino C₁₋₆ alkyl, acetylene, CHR₂₃R₂₄, COR₂₃, acetyl, NHCOCH₃, C₃₋₆ heterocyclyl, (C₃₋₆ heterocyclyl) C₁₋₆ alkylene, cyano, NHSO₂CH₃, N(SO₂CH₃)₂, carboxy, C₁₋₆ alkoxycarbonyl, amidoxime, trihalo C₁₋₆ alkoxy, oxo, hydroxyiminomethyl, C₁₋₆ alkylcarboxy, carboxy C₁₋₆ alkyl, trihaloacetyl, and methylsulfonyl;
wherein each of R₂₁ and R₂₂ is independently selected from H, C₁₋₆ alkyl, C₄₋₇ cycloalkyl, phenyl, benzyl, C₁₋₆ alkoxy, hydroxy, C₁₋₆ alkylamino, di(C₁₋₆)alkylamino, C₂₋₈ acyl, C₁₋₈ alkylsulfonyl;
R₂₃ is C₁₋₆ alkyl, C₄₋₇ cycloalkyl, phenyl, benzyl, C₁₋₆ alkoxy, hydroxy, aryl, C₁₋₆ alkylamino, di(C₁₋₆)alkylamino, C₂₋₈ acyl, C₁₋₈ alkylsulfonyl;
R₂₄ is H, halogen, hydroxy, amino, C₁₋₆ alkyl, C₄₋₇ cycloalkyl, phenyl, or benzyl;
in addition, said R₁, R₂ or R₃ that is a ring moiety is optionally substituted with between 1 and 3 substituents Q₁, Q₂, and Q₃, which, if present, are independently selected from: R₂₅, NR₂₆R₂₇, NHCOR₂₈, NHSOR₂₉, and NHSO₂R₃₀;
wherein R₂₅ is H, C₁₋₆ alkyl, C₄₋₇ cycloalkyl, phenyl, benzyl, C₁₋₆ alkoxy, hydroxy, C₁₋₆ alkylamino, di(C₁₋₆)alkylamino, C₂₋₈ acyl, or C₁₋₈ alkylsulfonyl;
wherein each of R₂₆ and R₂₇ is independently selected from H, C₁₋₆ alkyl, C₄₋₇ cycloalkyl, phenyl, benzyl, C₁₋₆ alkoxy, hydroxy, C₁₋₆ alkylamino, di(C₁₋₆)alkylamino, C₂₋₈ acyl, C₁₋₈ alkylsulfonyl;
each of R₂₈, R₂₉, and R₃₀ is C₁₋₆ alkyl, C₄₋₇ cycloalkyl, phenyl, benzyl, C₁₋₆ alkoxy, hydroxy, C₁₋₆ alkylamino, di(C₁₋₆)alkylamino, C₂₋₈ acyl, C₁₋₈ alkylsulfonyl;
and
R₁₁, R₁₂, R₁₄ and R₁₅ are each independently selected from hydrogen, halogen, C₁₋₆ alkyl and C₁₋₆ alkoxy;
R₁₃ is selected from hydrogen, oxo, and phenyl;
R₁₆ is selected from hydrogen, cyano, C₁₋₆ alkyl, and C₁₋₆ alkylamino;
wherein each of the above carbocyclyl and heterocarbocyclyls can be optionally substituted with between 1 and 3 substituents selected from C₁₋₄ alkyl, hydroxy, amino, halo, C₁₋₄ alkoxy, CONH₂, phenyl, and C₁₋₄ alkylamino, di(C₁₋₄)alkylamino;
and wherein —X—W-Z is not [4-(imidazol-1yl)-phenyl]oxy where a is 1 and b is 0;
or a pharmaceutically acceptable salt, ester, or amide thereof

2. The compound of claim 1, wherein Y is N.

3. The compound of claim 1, wherein a is 1 and b is 0.

4. The compound of claim 1, wherein a is 0 and b is 0.

5. The compound of claim 1, wherein a is 1 and b is 1.

6. The compound of claim 1, wherein at least two of R₁₁, R₁₂, R₁₃, and R₁₆ are H.

7. The compound of claim 1, wherein, if present, R₁₄ and R₁₅ are H.

8. The compound of claim 1, wherein one of R₁ and R₂ is a substituted ring.

9. The compound of claim 1, wherein R₁ is a substituted ring.

10. The compound of claim 1, wherein R₂ is a substituted ring.

11. The compound of claim 1, wherein one of R₁ and R₂ is a substituted phenyl or substituted pyridyl; and the other two of R₁, R₂ and R₃ are independently selected from hydrogen, halogen, and C₁₋₆ alkyl; wherein the substituent on said substituted phenyl or pyridyl is a para- or meta-substituent.

12. The compound of claim 1, wherein the substituent on said ring is of formula: X-Z or X—(C₁₋₆ alkylene)-Z, wherein X is selected from the group consisting of O, S, NR₂₁, —OCH₂—C≡C—, —NR₂₁—CO—, —CO—NR₂₁—, —NH—SO₂—, —SO₂—NH—, —NR₂₃—SO₂—, and —SO₂—NR₂₃; and Z is selected from (i) NR₂₁R₂₂ and pyridyl, piperidyl, and pyrrolidyl, optionally substituted.

13. The compound of claim 1, wherein a is 1 and b is 0; Y is N; one of R₁ and R₂ is phenyl para-substituted with X—W-Z, wherein X is O, NH, N(C₁₋₃ alkyl), NHCO, NHSO₂, or S; and W is C₂₋₅ alkylene.

14. The compound of claim 13, wherein Z is piperidyl or pyrrolidyl, optionally substituted with methyl, CONH₂, or phenyl.

15. The compound of claim 14, wherein R₁₁, R₁₂, R₁₃, and R₃ are each H.

16. The compound of claim 1, wherein each of R₃, R₁₁, R₁₂, and R₁₃ is H, halo, methyl, or methoxy.

17. The compound of claim 1, wherein the R₁, R₂, or R₃ that is a ring moiety is substituted with a moiety of formula —X—W-Z, —X-Z, or —W-Z.

18. The compound of claim 1, selected from (S, S)-3-(4-(3-Piperidinylpropoxy)phenyl)octahydroindolizine; (R, R)-3-(4-(3-Piperidinylpropoxy)phenyl)octahydroindolizine; trans-3-(4-(3-Piperidinylpropoxy)phenyl)octahydroindolizine; anti-2-(4-(3-Piperidinylpropoxy)phenyl)octahydroindolizine; syn-2-[4-(3-Piperidinylpropanoxy)phenyl]octahydroindolizine; 3-[4-(Piperidinylpropoxy)phenyl]hexahydro-1H-pyrrolizine; 5-[4-(4-Piperidinylbutoxy)phenyl]indolizine; trans-3-[4-(N-5-Piperidylpentylamino)phenyl]octahydroindolizine; 5-[4-(3-Piperidinylpropoxy)phenyl]octahydroindolizine; 5-[4-(4-Piperidinylpentanoxy)phenyl]octahydroindolizine; N-Methyl-N-[4-(trans-Octahydro-3-indolizinyl)phenyl]-3-piperidinylpropenamide; trans-3-[4-(N-3-Piperidylpropylamino)phenyl]octahydroindolizine; trans-3-[4-(3-Piperidinylmethylpropargyloxy)phenyl]octahydroindolizine; trans-3-[4-(N-5-Piperidylpentanamido)phenyl]octahydroindolizine; trans-3-{4-[2,2′-(N-Methylpyrrolidinyl)ethoxy]phenyl}octahydroindolizine; anti-2-[3-(3-Piperidinylpropyloxy)phenyl]octahydroindolizine; trans-3-[4-(N-4-Piperidylbutanamido)phenyl]octahydroindolizine; trans-3-[4-(N-Methyl-N-3-piperidylpropylamino)phenyl]octahydroindolizine; trans-3-[4-(3-Piperidylsulfonylamino)phenyl]octahydroindolizine; 5-[4-(2-Piperidinylethanoxy)phenyl]octahydroindolizine; trans-3-{4-[2,2′-(N-Methylpiperidinyl)ethoxy]phenyl}octahydroindolizine; tran-3-[4-(4-Methylaminophenylthio)phenyl]octahydroindolizine; trans-3-[4-(N-Methyl-N-5-piperidylpentylamino)phenyl]octahydroindolizine; 3-[4-(2-Piperidin-1-yl-ethoxy)-phenyl]-octahydro-indolizine; Dimethyl-{3-[4-(octahydro-indolizin-3-yl)-phenoxy]-propyl}-amine; trans-3-[4-(N-3-Piperidinylpropanamido)phenyl]octahydroindolizine; trans-3-{4-[(2-Piperidylethyl)sulfonyl]amidophenyl}octahydroindolizine; trans-3-{4-[(2-Piperidylethyl)sulfonyl-N-methylamino]phenyl}octahydroindolizine; and tran-3-[4-(4-Carboxylicphenylthio)phenyl]octahydroindolizine.

19. The compound of claim 1, selected from: trans-3-[4-((4-Amidoxime)phenylthio)phenyl]octahydroindolizine; trans-3-[4-(4-Methansulfonaminophenoxy)phenyl]octahydroindolizine; trans-3-{4-[2,2′-(N-Trifluoroethylpiperidinyl)ethoxy]phenyl}octahydroindolizine; trans-3-{4-[2,2′-(1-tert-Butylcarboxylatepiperidinyl)ethoxy]phenyl}-octahydroindolizine; trans-3-[4-(3-Piperidylsulfonyl-N-methylamino)phenyl]octahydroindolizine; trans-3-[4-(4-Aminophenylthio)phenyl]octahydroindolizine; trans-3-[4-(N-Methyl-N-5-piperidylpentanamido)phenyl]octahydroindolizine; Octahydro-3-[4-(4-pyridinylthio)phenyl]indolizine; trans-3-[4-(N-Phenyl-1-piperazinylmethyl)phenyl]octahydroindolizine; trans-3-[4-(4-Pyridinylethenyl)phenyl]octahydroindolizine; trans-3-{4-[2,2′-(N-Trifluoroacetylpiperidinyl)ethoxy]phenyl}octahydroindolizine; tran-3-[4-(3-(2-Dimethylaminoethyl)amino)phenyl]octahydroindolizine; trans-3-[4-(4-Pyridyloxy)phenyl]octahydroindolizine; trans-3-{4-[2,2′-(N-Amidinopiperidinyl)ethoxy]phenyl}octahydroindolizine; trans-3-[4-(4-Pyridylmethan-1-ol)phenyl]octahydroindolizine; trans-3-[4-(2,2′-piperidinylethoxy)phenyl]octahydroindolizine; 4-[4-(Octahydro-indolizin-3-yl)-phenoxy]-quinazoline; trans-3-[4-(N-Methylsulfonyl)piperidinylamino)phenyl]octahydroindolizine; trans-3-[4-(3-bis-Methansulfonaminobenzyloxy)phenyl]octahydroindolizine; 3-(4-Thiophen-2-yl-phenyl)-octahydro-indolizine; trans-3-[4-(N-Methylsulfonyl-4-aminopiperidine)phenyl]octahydroindolizine; 4-[4-(4-Pyridylthio)phenyl]octahydroquinolizine; trans-3-[4-(3-Methansulfonaminobenzyloxy)phenyl]octahydroindolizine; and trans-3-[4-(4-Trifluoromethoxyphenyl)phenyl]octahydroindolizine.

20. The compound of claim 1, selected from:
3-Biphenyl-4-yl-octahydro-indolizine; trans-3-(4-Phenoxy-phenyl)-octahydro-indolizine; cis-3-(4-Phenoxy-phenyl)-octahydro-indolizine; Dimethyl-[5-(octahydro-indolizin-3-yl)-naphthalen-1-yl]-amine; [4-(Octahydro-indolizin-3-yl)-phenyl]-diphenyl-amine; 5-[4-(4-Pyridinylthio)phenyl]octahydroindolizine; 5-[4-(4-Nitrophenylthio)phenyl]octahydroindolizine; 3-[4-(Pyridin-3-yloxy)-phenyl]-octahydro-indolizine; 2-[4-(Octahydro-indolizin-3-yl)-phenoxy]-1H-benzoimidazole; 3-[4-(4-Nitro-phenylsulfanyl)-phenyl]-octahydro-indolizine; 3-[4-(Pyrimidin-2-ylsulfanyl)-phenyl]-octahydro-indolizine; 2-[4-(Octahydro-indolizin-3-yl)-phenylsulfanyl]-3H-quinazolin-4-one; 2-[4-(Octahydro-indolizin-3-yl)-phenoxy]-quinoline; 2-Methyl-8-[4-(octahydro-indolizin-3-yl)-phenoxy]-quinoline; 4-[4-(Octahydro-indolizin-3-yl)-phenylsulfanyl]-benzonitrile; 5-(4-(4-Aminophenylthio)phenyl)octahydroindolizine; 3-Methylamino-3-(4-bromophenyl)octahydroindolizine; trans-3-[4-(4-Methylene-1,3-thiazolidine-2,4-diimine)phenyl]octahydroindolizine; 4′-(Octahydro-indolizin-3-yl)-biphenyl-3-ylamine; 3-(4-Thiophen-3-yl-phenyl)-octahydro-indolizine; 2-[4-(Octahydro-indolizin-3-yl)-phenyl]-thiophene-3-carbaldehyde; 4′-(Octahydro-indolizin-3-yl)-biphenyl-4-carbaldehyde; 3-(4′-Fluoro-biphenyl-4-yl)-octahydro-indolizine; and trans-3-[4-(3-hydroxyiminomethylthienyl)phenyl]octahydroindolizine.

21. The compound of claim 1, selected from: trans-3-[4-(3-Methylsulfonylaminophenyl)phenyl]octahydroindolizine; anti-2-[2-(3-Piperidinylpropoxy)phenyl]octahydroindolizine; trans-3-[4-(4-Aminophenoxy)phenyl]octahydroindolizine; trans-3-(4-Aminophenyl)octahydroindolizine; trans-3-(4-(N,N-Dimethylamino)phenyl)octahydroindolizine; trans-3-(4-(Methylsulfonylamino)phenyl)octahydroindolizine; trans-3-(4-(bis-Methylsulfonylamino)phenyl)octahydroindolizine; trans-3-{4-[4-(N-(1,1-dimethylethoxycarbonyl)piperidinylamino]phenyl}octahydroindolizine; trans-3-[4-(4-Piperidinylamino)phenyl]octahydroindolizine; trans-3-[4-(N-Ethyl-N-4-N-methylsulfonylpiperidinylamino)phenyl]octahydroindolizine; N-[4-(trans-Octahydro-3-indolizinyl)phenyl]propenamide; N-Methyl-N-[4-(trans-Octahydro-3-indolizinyl)phenyl]propenamide; and trans-3-{4-[(2-Pyrrolidylethyl)sulfonylamino]phenyl}octahydroindolizine.

22. The compound of claim 1, selected from: trans-3-{4-[(4-Chlorophenyl)methan-1-ol]phenyl}octahydroindolizine; trans-3-{4-[(4-Chlorobenzyl]phenyl}octahydroindolizine; [4-(Octahydro-indolizin-3-yl)-phenyl]-pyridin-3-ylmethyl-amine; [4-(Octahydro-indolizin-3-yl)-phenyl]-pyridin-2-ylmethyl-amine; [4-(Octahydro-indolizin-3-yl)-phenyl]-thiophen-3-ylmethyl-amine; Furan-2-ylmethyl-[4-(octahydro-indolizin-3-yl)-phenyl]-amine; [4-(Octahydro-indolizin-3-yl)-phenyl]-pyridin-4-ylmethyl-amine; Benzyl-[4-(octahydro-indolizin-3-yl)-phenyl]-amine; [4-(Octahydro-indolizin-3-yl)-phenyl]-(1-oxy-pyridin-4-ylmethyl)-amine; (1H-Imidazol-2-ylmethyl)-[4-(octahydro-indolizin-3-yl)-phenyl]-amine; Dibenzyl-[4-(octahydro-indolizin-3-yl)-phenyl]-amine; (R, R)-Octahydro-3-[4-(4-pyridinylthio)phenyl]indolizine; and (S, S-Octahydro-3-[4-(4-pyridinylthio)phenyl]indolizine.

23. A pharmaceutical composition comprising a pharmaceutically acceptable carrier and a compound of claim 1.

24. A method for treating a disorder or condition mediated by the histamine H₃ receptor in a subject, said method comprising administering to a subject a therapeutically effective amount of a compound of claim 1.

25. A method of claim 24, wherein said disorder or condition is selected from the group consisting of sleep/wake disorders, arousal/vigilance disorders, migraine, asthma, dementia, mild cognitive impairment (pre-dementia), Alzheimer's disease, epilepsy, narcolepsy, eating disorders, motion sickness, vertigo, attention deficit hyperactivity disorders, learning disorders, memory retention disorders, schizophrenia, nasal congestion, allergic rhinitis, and upper airway allergic response.

26. A method for treating a disease or condition modulated by at least one receptor selected from the histamine H₁ receptor and the histamine H₃ receptor, said method comprising (a) administering to a subject a jointly effective amount of a histamine H₁ receptor antagonist compound, and (b) administering to the subject a jointly effective amount of a compound of claim 1, said method providing a jointly therapeutically effective amount of said compounds.

27. (canceled)

28. A method for treating diseases or conditions modulated by at least one receptor selected from the histamine H₂ receptor and the histamine H₃ receptor in a subject, comprising (a) administering to the subject a jointly effective amount of a histamine H₂ receptor antagonist compound, and (b) administering to the subject a jointly effective amount of a compound of claim 1, said method providing a jointly therapeutically effective amount of said compounds.

29. (canceled)

30. A method for treating one or more disorders or conditions selected from the group consisting of sleep/wake disorders, narcolepsy, and arousal/vigilance disorders, comprising administering to a subject a therapeutically effective amount of a compound of claim 1.

31. A method for treating attention deficit hyperactivity disorders (ADHD), comprising administering to a subject a therapeutically effective amount of a compound of claim 1.

32. A method for treating one or more disorders or conditions selected from the group consisting of dementia, mild cognitive impairment (pre-dementia), cognitive dysfunction, schizophrenia, depression, manic disorders, bipolar disorders, and learning and memory disorders, comprising administering to a subject a therapeutically effective amount of a compound of claim 1.

33. A method for treating or preventing upper airway allergic response, nasal congestion, or allergic rhinitis, comprising administering to a subject a therapeutically effective amount of a compound of claim 1.

34. A method for studying disorders mediated by the histamine H₃ receptor, comprising using a ¹¹C- or ¹⁸F-labeled compound of claim 1 as a positron emission tomography (PET) molecular probe.
